SEQUENCE LISTING (97 sequences)

SEQ ID NO: 1 - protein, 196 aa, Artificial Sequence
  H1
MSKKGIDVSEWQGDIDFNAVKASGVEFVIIRAGYGIGCKDKWFEQNYRKAKTAGLDVGAYWYSYANSGFEAAEEAQSLMNMLSGKSFEYPVYFDLEEKSQLNRGRAFCDSLITSFCNKLEACGYYAGFYTSLSTANNLVSAHVRNRYALWIAQWNTHCNYQGSYGLWQYSSNGSVPGVAGRVDMDYAYVDYPSIIK

SEQ ID NO: 2 - protein, 196 aa, Artificial Sequence
  H2
MSKRGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITGFCNKLESCGYYAGFYTSLSTANNLVSAHVRNRYALWIAQWNTHCSYQGSYGLWQYSSSGSVPGVAGRVDMDYAYVDYPSIIK

SEQ ID NO: 3 - protein, 196 aa, Artificial Sequence
  H3
MSKKGIDVSVWQGDIDFNSVKASGVEFVIIRAGYGIGHKDKWFEENYRKAKTAGLDVGSYWYSYASSAGEASEEAQSCVNILSGKSFEYPIYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIK

SEQ ID NO: 4 - protein, 196 aa, Artificial Sequence
  H4
MSKKGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITSFCSKLETYGYYAGFYTSLSVVNNLVSAHVRDRYALWIAQWNTHCSYQGSYGLWQYSSSGSVPGVAGRVDMDYAYVDYPSIIK

SEQ ID NO: 5 - protein, 196 aa, Artificial Sequence
  H5
MSKKGIDVSEWQGDIDFNAVKASGVEFVIIRAGYGIGCKDKWFEQNYRKAKTAGLDVGAYWYSYANSSSEAAEEAQSCANMLSGKSFEYPVYFDLEEKSQLNRGRAFCDSLITGFCSKLEACGYYAGFYTSLSTANNLVSAHVRNRYALWIAQWNTHCSYQGSYGLWQYSSNGSVPGVAGRVDMDYAYKDYPSIIK

SEQ ID NO: 6 - protein, 196 aa, Artificial Sequence
  H6
MSKKGIDVSVWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITSFCSKLESCGYYAGFYTSLSTANNLVSAHVRNRYALWIAQWNTHCDYQGSYGLWQYSSSGSVPGVAGRVDMDYAYKNYPSIIK

SEQ ID NO: 7 - protein, 196 aa, Artificial Sequence
  H7
MSKKGIDVSEWQGDIDFNAVKASGVEFVIIRAGYGIGCKDKWFEQNYRKAKTAGLDVGAYWYSYANSASEAAEEAQSCANMLSGKSFEYPVYFDLEEKSQLNRGRAFCDSLITSFCSKLETYGYYAGFYTSLSTANNLVSSHVRNRYALWIAQWNTHCSYQGSYGLWQYSSSGSVPGVAGRVDMDYAYKDYPSIIK

SEQ ID NO: 8 - protein, 306 aa, Artificial Sequence
  EL8
MSKKGIDVSEWQGDIDFNAVKASGVEFVIIRAGYGIGCKDKWFEQNYRKAKTAGLDVGAYWYSYANSSSEAAEEAQSCVNMLSGKSFEYPVYFDLEEKSQLNRGRAFCDSLITSFCSKLETYGYYAGFYTSLSTANNLVSSHVRNRYALWIAQWNTHCSYQGSYGLWQYSSNGSVPGVAGRVDMDYAYVDYPSIIKNAGLNGYKNGGSYTAPQTSSIDDVAREVINGAWGNGNERKQRLTQAGYDYTSVQNKVNKLLGVKACRKSVDELAREVIRGTWGNGNERKNRLTQAGYDYDTVQKRVNELL

SEQ ID NO: 9 - protein, 251 aa, Artificial Sequence
  EL9
MSKKGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRVFCDSLITSFCNKLEACGYYAGFYTSLSTANNLVSSHVRNRYALWIAQWNTHCDYQGSYGLWQYSSSGSVPGVAGRVDMDYAYVDYPSIIKNAGLNGCKNGGSDQAARTSSIDEVAREVINGAWGNGSTRKQRLTSAGYDYASVAK

SEQ ID NO: 10 - protein, 196 aa, Artificial Sequence
  H10
MSKRGIDVSVWQGDIDFNAVKASGVEFVIIRAGYGIGHKDKWFEENYRKAKTVGLDVGAYWYSYASSAGEASEEAQSCVNILSGKSFEYPVYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIK

SEQ ID NO: 11 - protein, 196 aa, Artificial Sequence
  H11
MSKRGIDVSVWQGDIDFNAVKASGVEFVIIRAGYGIGHKDKWFEQNYRKAKTTGLDVGAYWYSYASSAGEAAEEAQSCVNILSGKSFEYPVYFDLEEKSQLNRGRDFCDSLITSFCNKLETYGYYAGFYTSLSVANNLVSSHVRDRYALWIAQWNTHCDYQGSYGLWQYSSSGSVDGIAGRVDMDYTYVDYPSVIK

SEQ ID NO: 12 - protein, 196 aa, Artificial Sequence
  H12
MSKKGIDVSVWQGDIDFNAVKASGVEFVIIRAGYGIGHKDKWFEENYRKAKTAGLDVGSYWYSYASSAGEVALEAQSCVNILSGKSFEYPVYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIK

SEQ ID NO: 13 - protein, 306 aa, Artificial Sequence
  EL13
MSKKGIDVSVWQGDIDFNAVKASGVEFVIIRAGYGIECKDKWFEQNYRKAKTAGLDVGAYWYSYANSGFEAAEEAQSCVNMLSGKSFEYPVYFDLEEKSQLNRGRAFCDSLITSFCNKLESCGYYAGFYTSLSTANNLVPAHVRNRYALWIAQWNTHCDYQGSYGLWQYSSSGSVPGVAGRVDMDYAYVDYPSIIKNAGLNGYKNGESHQATRTTSIDEVAREVINGAWGNGNERKQRLTQAGYDYASVQNKVNELLGVKACRKSVDELAREVIRGTWGNGNERKNRLTSAGYDYDTVQKRVNELL

SEQ ID NO: 14 - protein, 306 aa, Artificial Sequence
  EL14
MSKKGIDVSVWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITSFCNKLEACGYYAGFYTSLSTANNLVSAHVRNRYALWIAQWNTHCSYQGSYGLWQYSSSGSVPGVAGRVDMDYAYVDYPSIIKNAGLNGYKNGESHQATRTTSIDEVAREVINGAWGNGNERKQRLTSAGYDYASVQNKVNELLGVKACRKSVDELAREVIRGAWGNGSTRKQRLTSAGYDYDTVQKRVNELL

SEQ ID NO: 15 - protein, 49 aa, Artificial Sequence
  B1_N
DQAARTSSIDEVAREVINGAWGNGSTRKQRLTSAGYDYASVQNKVNELL

SEQ ID NO: 16 - protein, 49 aa, Artificial Sequence
  B1_C
GVKACRKSVDELAREVIRGAWGNGSTRKQRLAQAGYDYDTVQKRVNELL

SEQ ID NO: 17 - protein, 49 aa, Artificial Sequence
  B2_N
DQAARTSSIDEVAREVINGAWGNGNERKQRLTSAGYDYASVQNKVNELL

SEQ ID NO: 18 - protein, 49 aa, Artificial Sequence
  B2_C
GVKACRKSVDEIAREVIRGTWGNGSTRKQRLTQAGYDYDTVQKRVNELL

SEQ ID NO: 19 - protein, 49 aa, Artificial Sequence
  B3_N
YTAPQTSSIDEVAREVINGDWGNGNDRKNRLISAGYDYASVQNKVNELL

SEQ ID NO: 20 - protein, 49 aa, Artificial Sequence
  B3_C
GVKAYRKSVDELAREVIRGTWGNGSMRKHRLTQAGYDYDAVQKRVNELL

SEQ ID NO: 21 - protein, 49 aa, Artificial Sequence
  B4_N
DQATRTSSIDEVAREVINGAWGNGNERKQRLTSAGYDYASVQNKVNKLL

SEQ ID NO: 22 - protein, 49 aa, Artificial Sequence
  B4_C
GVKAYRKSVDELAREVIRGTWGNGNERKQRLAQAGYDYDTVQKRVNELL

SEQ ID NO: 23 - protein, 49 aa, Artificial Sequence
  B5_N
DQAARTSSIDEVAREVINGAWGNGNERKQRLTQAGYDYTSVQNKVNKLL

SEQ ID NO: 24 - protein, 49 aa, Artificial Sequence
  B5_C
GVKACRKSVDELAREVIRGTWGNGNERKNRLTQAGYDYDTVQKRVNELL

SEQ ID NO: 25 - protein, 43 aa, Artificial Sequence
  B6_N
NQAARTSSIDDVAREVINGAWGNGNERKQRLTQAGYDYASVAK

SEQ ID NO: 26 - protein, 49 aa, Artificial Sequence; identical to SEQ ID NO: 23
  B7_N

SEQ ID NO: 27 - protein, 49 aa, Artificial Sequence; identical to SEQ ID NO: 24
  B7_C

SEQ ID NO: 28 - protein, 49 aa, Artificial Sequence
  B10_N
YTAPQISSIDEVAREVINGDWGNGNERKQRLTSAGYDYASVQNKVNELL

SEQ ID NO: 29 - protein, 49 aa, Artificial Sequence
  B10_C
GVKAYRKSVDELAREVIRGTWGNGSTRKQRLTQAGYDYNAVQKRVNELL

SEQ ID NO: 30 - protein, 49 aa, Artificial Sequence
  B11_N
YTAPQTSSIDEVAREVINGDWGNGNERKNRLTSAGYDYTSVQNKVNELL

SEQ ID NO: 31 - protein, 49 aa, Artificial Sequence
  B11_C
GVKAYRKSVDELAREVIRGTWGNGSTRKQRLTQAGYDYDAVQKRVNELL

SEQ ID NO: 32 - protein, 49 aa, Artificial Sequence
  B12_N
YTAPQTSSIDEVAREVINGDWGNGIERKNRLTSAGYDYTSVQNKVNELL

SEQ ID NO: 33 - protein, 49 aa, Artificial Sequence
  B12_C
GVKAYRKSVDELAREVIRGTWGNGKTRKQRLTQAGYDYNAVQKRVNELL

SEQ ID NO: 34 - protein, 12 aa, Artificial Sequence
  linker polypeptide
  VARIANT 1: Asn = Lys
  VARIANT 8: Xaa = Lys or Gln
NAGLNGYXNGGS

SEQ ID NO: 35 - protein, 6 aa, Artificial Sequence
  linker
  VARIANT 1..6: Xaa = Gly, Ala or Ser
XXXXXX

SEQ ID NO: 36 - protein, 9 aa, Artificial Sequence
  linker
  VARIANT 1..9: Xaa = Gly, Ala or Ser
XXXXXXXXX

SEQ ID NO: 37 - protein, 12 aa, Artificial Sequence
  linker
  VARIANT 1..12: Xaa = Gly, Ala or Ser
XXXXXXXXXXXX

SEQ ID NO: 38 - protein, 15 aa, Artificial Sequence
  linker
  VARIANT 1..15: Xaa = Gly, Ala or Ser
XXXXXXXXXXXXXXX

SEQ ID NO: 39 - protein, 18 aa, Artificial Sequence
  linker
  VARIANT 1..18: Xaa = Gly, Ala or Ser
XXXXXXXXXXXXXXXXXX

SEQ ID NO: 40 - protein, 6 aa, Artificial Sequence
  linker
GGSGGS

SEQ ID NO: 41 - protein, 9 aa, Artificial Sequence
  linker
GGSGGSGGS

SEQ ID NO: 42 - protein, 12 aa, Artificial Sequence
  linker
GGSGGSGGSGGS

SEQ ID NO: 43 - protein, 15 aa, Artificial Sequence
  linker
GGSGGSGGSGGSGGS

SEQ ID NO: 44 - protein, 18 aa, Artificial Sequence
  linker
GGSGGSGGSGGSGGSGGS

SEQ ID NO: 45 - protein, 12 aa, Artificial Sequence
  linker
NAGLNGYKNGGS

SEQ ID NO: 46 - protein, 12 aa, Artificial Sequence
  linker
  VARIANT 1: Xaa = Asn or Lys
  VARIANT 2: Xaa = Ala or Val
  VARIANT 7: Xaa = Tyr or Cys
  VARIANT 8: Xaa = Lys or Gln
XXGLNGXXNGGS

SEQ ID NO: 47 - protein, 39 aa, Artificial Sequence
  CW_7 motif
  VARIANT 38: Xaa is Ile for motif No. 1 and No. 2 Xaa is Leu for motif No. 3
TVANEVIQGLWGNGQERYDSLANAGYDPQAVQDKVNEXL

SEQ ID NO: 48 - DNA, 42 bp, Artificial Sequence
  primer T7casette-16-rev
aacaggtctcaatacaatccggatatagttcctcctttcagc

SEQ ID NO: 49 - DNA, 43 bp, Artificial Sequence
  primer T7casette-01-for
aacaggtctcaacctccgcgaaattaatacgactcactatagg

SEQ ID NO: 50 - DNA, 37 bp, Artificial Sequence
  primer EL_H1-rev
aacaggtctcaagccggcatttttgatgatgctcggg

SEQ ID NO: 51 - DNA, 43 bp, Artificial Sequence
  primer EL_H2-rev
aacaggtctcaagccaacatttttaataatgctcggataatcc

SEQ ID NO: 52 - DNA, 37 bp, Artificial Sequence
  primer EL_H3-rev
aacaggtctcaagccggcgtttttgataacgctcggg

SEQ ID NO: 53 - DNA, 36 bp, Artificial Sequence
  primer EL_H4-rev
aacaggtctcaagcccacgttcttgatgatgctcgg

SEQ ID NO: 54 - DNA, 36 bp, Artificial Sequence
  primer EL_H5-rev
aacaggtctcaagccggcgtttttgatgatgctcgg

SEQ ID NO: 55 - DNA, 39 bp, Artificial Sequence
  primer EL_H6-rev
aacaggtctcaagccggcattcttaataatgctcggata

SEQ ID NO: 56 - DNA, 42 bp, Artificial Sequence
  primer EL_H7-rev
aacaggtctcaagccggcgtttttaatgatgctcggataatc

SEQ ID NO: 57 - DNA, 36 bp, Artificial Sequence
  primer EL_H10-rev
aacaggtctcaagccggcgtttttgatcacgctcgg

SEQ ID NO: 58 - DNA, 42 bp, Artificial Sequence
  primer EL_H11-rev
aacaggtctcaagccggccttcttgataacgctcggataatc

SEQ ID NO: 59 - DNA, 36 bp, Artificial Sequence
  primer EL_H12-rev
aacaggtctcaagccggcattcttgatcacgctcgg

SEQ ID NO: 60 - DNA, 37 bp, Artificial Sequence
  primer EL_B6-for
aacaggtctcaggcttaaacggctgcaaaaatggcgg

SEQ ID NO: 61 - DNA, 37 bp, Artificial Sequence
  primer EL_B7-for
aacaggtctcaggcttaaacggctgcaaaaacggtgg

SEQ ID NO: 62 - DNA, 38 bp, Artificial Sequence
  primer EL_B10-for
aacaggtctcaggcttaaacggctataaaaacggcggc

SEQ ID NO: 63 - DNA, 40 bp, Artificial Sequence
  primer EL_B11-for
aacaggtctcaggcttaaatggttacaagaatggcggcag

SEQ ID NO: 64 - DNA, 37 bp, Artificial Sequence
  primer EL_B12-for
aacaggtctcaggcttaaatggctaccagaacggcgg

SEQ ID NO: 65 - DNA, 37 bp, Artificial Sequence
  primer EL_B1-for
aacaggtctcaggcttaaacggctgcaagaatggtgg

SEQ ID NO: 66 - DNA, 37 bp, Artificial Sequence
  primer EL_B2-for
aacaggtctcaggcttaaatggttgcaagaacggcgg

SEQ ID NO: 67 - DNA, 38 bp, Artificial Sequence
  primer EL_B3-for
aacaggtctcaggcttaaatggctaccagaatggcggc

SEQ ID NO: 68 - DNA, 38 bp, Artificial Sequence
  primer EL_B4-for
aacaggtctcaggcttaaatggctgcaaaaacggtggc

SEQ ID NO: 69 - DNA, 37 bp, Artificial Sequence
  primer T7term-STOP-for
aacaggtctcatgacgccattaacctgatgttctggg

SEQ ID NO: 70 - protein, 12 aa, Artificial Sequence
  linker region L10
NAGLNGYKNGGS

SEQ ID NO: 71 - protein, 5 aa, Artificial Sequence
  C-terminus of B-domains
VNELL

SEQ ID NO: 72 - protein, 5 aa, Artificial Sequence
  C-terminus of B-domains
VNKLL

SEQ ID NO: 73 - protein, 5 aa, Artificial Sequence
  C-terminus of CW_7 motifs
VNEIL

SEQ ID NO: 74 - protein, 12 aa, Artificial Sequence
  L1
NAGLNGCKNGGS

SEQ ID NO: 75 - protein, 12 aa, Artificial Sequence
  L2
NVGLNGCKNGGS

SEQ ID NO: 76 - protein, 12 aa, Artificial Sequence
  L3
NAGLNGYQNGGS

SEQ ID NO: 77 - protein, 12 aa, Artificial Sequence
  L4
NVGLNGCKNGGS

SEQ ID NO: 78 - protein, 12 aa, Artificial Sequence
  L5
NAGLNGCKNGGS

SEQ ID NO: 79 - protein, 12 aa, Artificial Sequence
  L6
NAGLNGCKNGGS

SEQ ID NO: 80 - protein, 12 aa, Artificial Sequence
  L7
NAGLNGCKNGGS

SEQ ID NO: 81 - protein, 12 aa, Artificial Sequence
  L10
NAGLNGYKNGGS

SEQ ID NO: 82 - protein, 12 aa, Artificial Sequence
  L11
KAGLNGYKNGGS

SEQ ID NO: 83 - protein, 12 aa, Artificial Sequence
  L12
NAGLNGYQNGGS

SEQ ID NO: 84 - protein, 306 aa, Artificial Sequence
  EL11_strain_JCP8017A
MSKRGIDVSVWQGDIDFNAVKASGVEFVIIRAGYGIGHKDKWFEQNYRKAKTTGLDVGAYWYSYASSAGEAAEEAQSCVNILSGKSFEYPVYFDLEEKSQLNRGRDFCDSLITSFCNKLETYGYYAGFYTSLSVANNLVSSHVRDRYALWIAQWNTHCDYQGSYGLWQYSSSGSVDGIAGRVDMDYTYVDYPSVIKKAGLNGYKNGGSYTAPQTSSIDEVAREVINGDWGNGNERKNRLTSAGYDYTSVQNKVNELLGVKAYRKSVDELAREVIRGTWGNGSTRKQRLTQAGYDYDAVQKRVNELL

SEQ ID NO: 85 - protein, 306 aa, Artificial Sequence
  EL3_strain_Gv18-4
MSKKGIDVSVWQGDIDFNSVKASGVEFVIIRAGYGIGHKDKWFEENYRKAKTAGLDVGSYWYSYASSAGEASEEAQSCVNILSGKSFEYPIYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIKNAGLNGYQNGGSYTAPQTSSIDEVAREVINGDWGNGNDRKNRLISAGYDYASVQNKVNELLGVKAYRKSVDELAREVIRGTWGNGSMRKHRLTQAGYDYDAVQKRVNELL

SEQ ID NO: 86 - protein, 306 aa, Artificial Sequence
  EL10_strain_G30-4
MSKRGIDVSVWQGDIDFNAVKASGVEFVIIRAGYGIGHKDKWFEENYRKAKTVGLDVGAYWYSYASSAGEASEEAQSCVNILSGKSFEYPVYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIKNAGLNGYKNGGSYTAPQISSIDEVAREVINGDWGNGNERKQRLTSAGYDYASVQNKVNELLGVKAYRKSVDELAREVIRGTWGNGSTRKQRLTQAGYDYNAVQKRVNELL

SEQ ID NO: 87 - protein, 306 aa, Artificial Sequence
  EL12_strain_3549624_non-prophage
MSKKGIDVSVWQGDIDFNAVKASGVEFVIIRAGYGIGHKDKWFEENYRKAKTAGLDVGSYWYSYASSAGEVALEAQSCVNILSGKSFEYPVYFDLEEKSQLNRGRDFCDSLITSFCNKLEACGYYAGFYTSLSVANNLVSSHVRDRYALWIAQWNTHCSYQGSYGLWQYSSSGSVNGIAGRVDMDYAYVDYPSVIKNAGLNGYQNGGSYTAPQTSSIDEVAREVINGDWGNGIERKNRLTSAGYDYTSVQNKVNELLGVKAYRKSVDELAREVIRGTWGNGKTRKQRLTQAGYDYNAVQKRVNELL

SEQ ID NO: 88 - protein, 306 aa, Artificial Sequence; identical to SEQ ID NO: 8
  EL8_strain_JCP7719

SEQ ID NO: 89 - protein, 306 aa, Artificial Sequence
  EL5_strain_JCP7276
MSKKGIDVSEWQGDIDFNAVKASGVEFVIIRAGYGIGCKDKWFEQNYRKAKTAGLDVGAYWYSYANSSSEAAEEAQSCANMLSGKSFEYPVYFDLEEKSQLNRGRAFCDSLITGFCSKLEACGYYAGFYTSLSTANNLVSAHVRNRYALWIAQWNTHCSYQGSYGLWQYSSNGSVPGVAGRVDMDYAYKDYPSIIKNAGLNGCKNGGSDQAARTSSIDEVAREVINGAWGNGNERKQRLTQAGYDYTSVQNKVNKLLGVKACRKSVDELAREVIRGTWGNGNERKNRLTQAGYDYDTVQKRVNELL

SEQ ID NO: 90 - protein, 306 aa, Artificial Sequence
  EL7_strain_AMD
MSKKGIDVSEWQGDIDFNAVKASGVEFVIIRAGYGIGCKDKWFEQNYRKAKTAGLDVGAYWYSYANSASEAAEEAQSCANMLSGKSFEYPVYFDLEEKSQLNRGRAFCDSLITSFCSKLETYGYYAGFYTSLSTANNLVSSHVRNRYALWIAQWNTHCSYQGSYGLWQYSSSGSVPGVAGRVDMDYAYKDYPSIIKNAGLNGCKNGGSDQAARTSSIDEVAREVINGAWGNGNERKQRLTQAGYDYTSVQNKVNKLLGVKACRKSVDELAREVIRGTWGNGNERKNRLTQAGYDYDTVQKRVNELL

SEQ ID NO: 91 - protein, 306 aa, Artificial Sequence
  EL4_strain_Gv5-1
MSKKGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITSFCSKLETYGYYAGFYTSLSVVNNLVSAHVRDRYALWIAQWNTHCSYQGSYGLWQYSSSGSVPGVAGRVDMDYAYVDYPSIIKNVGLNGCKNGGSDQATRTSSIDEVAREVINGAWGNGNERKQRLTSAGYDYASVQNKVNKLLGVKAYRKSVDELAREVIRGTWGNGNERKQRLAQAGYDYDTVQKRVNELL

SEQ ID NO: 92 - protein, 251 aa, Artificial Sequence
  EL6_strain_1400E_frameshift
MSKKGIDVSVWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITSFCSKLESCGYYAGFYTSLSTANNLVSAHVRNRYALWIAQWNTHCDYQGSYGLWQYSSSGSVPGVAGRVDMDYAYKNYPSIIKNAGLNGCKNGGSNQAARTSSIDDVAREVINGAWGNGNERKQRLTQAGYDYASVAK

SEQ ID NO: 93 - protein, 306 aa, Artificial Sequence
  EL1_strain_HMP9231
MSKKGIDVSEWQGDIDFNAVKASGVEFVIIRAGYGIGCKDKWFEQNYRKAKTAGLDVGAYWYSYANSGFEAAEEAQSLMNMLSGKSFEYPVYFDLEEKSQLNRGRAFCDSLITSFCNKLEACGYYAGFYTSLSTANNLVSAHVRNRYALWIAQWNTHCNYQGSYGLWQYSSNGSVPGVAGRVDMDYAYVDYPSIIKNAGLNGCKNGGSDQAARTSSIDEVAREVINGAWGNGSTRKQRLTSAGYDYASVQNKVNELLGVKACRKSVDELAREVIRGAWGNGSTRKQRLAQAGYDYDTVQKRVNELL

SEQ ID NO: 94 - protein, 251 aa, Artificial Sequence; identical to SEQ ID NO: 9
  EL9_strain_0288E_incomplete_contig

SEQ ID NO: 95 - protein, 306 aa, Artificial Sequence
  EL2_strain_Gv18-4
MSKRGIDVSEWQGDIDFNAVKASGVEFVIIRAGYGIGCKDKWFEQNYRKAKTCGLDVGAYWYSYANSGFEAAEEAQSCVNMLSGKSFEYPVYFDLEEKSQLNRGRAFCDSLITGFCNKLESCGYYAGFYTSLSTANNLVSAHVRNRYALWIAQWNTHCSYQGSYGLWQYSSSGSVPGVAGRVDMDYAYVDYPSIIKNVGLNGCKNGGSDQAARTSSIDEVAREVINGAWGNGNERKQRLTSAGYDYASVQNKVNELLGVKACRKSVDEIAREVIRGTWGNGSTRKQRLTQAGYDYDTVQKRVNELL

SEQ ID NO: 96 - protein, 306 aa, Artificial Sequence; identical to SEQ ID NO: 13
  EL13_strain_Gv37_1

SEQ ID NO: 97 - protein, 306 aa, Artificial Sequence; identical to SEQ ID NO: 14
  EL14_strain_Gv37_2

(19) United States
(12) Patent Application Publication
(10) Pub. No.: US 2022/0315908 A1
(43) Pub. Date: Oct. 6, 2022

(54) NOVEL GARDNERELLA ENDOLYSINS AND USES THEREOF

(71) Applicant: PHAGOMED BIOPHARMA GMBH, Wien (AT)

(72) Inventor: Lorenzo CORSINI, Wien (AT)

(73) Assignee: PHAGOMED BIOPHARMA GMBH, Wien (AT)

(21) Appl. No.: 17/609,358

(22) PCT Filed: May 7, 2020
(86) PCT No.: PCT/EP2020/062645

§ 371 Date: Nov. 5, 2021

(30) Foreign Application Priority Data
May 8, 2019 (EP) 19173389.8

(51) Int. Cl. C12N 9/36; C12N 9/80; C12N 9/52; C07K 14/005; G01N 33/569; C12N 15/62; A61P 31/04

(52) U.S. Cl. CPC C12N 9/2462; C12N 9/80; C12N 9/52; C07K 14/005; G01N 33/56911; C12N 15/62; A61P 31/04; C12N 2795/00022; A61K 38/00

(57) ABSTRACT

The present invention relates to new species-selective phage endolysins and their use to treat bacterial vaginosis (BV). The present invention provides recombinant endolysins, i.e. domain-swapped endolysins. The invention also relates to said endolysins for use in treating diseases or disorders such as bacterial infections, in particular BV. The invention further relates to polynucleotides encoding said endolysins. Said polynucleotides can also be used for treating such diseases or disorders. Also provided by the present invention is a pharmaceutical composition comprising an endolysin of the invention for use in treating such diseases or disorders. Said endolysins, polynucleotides and pharmaceutical composition may be administered locally, in particular locally into the vagina.

### RELATED APPLICATIONS

The present application is a National Stage entry of International Patent Application No. PCT/EP2020/062645, filed May 7, 2020, which claims priority to European Patent Application No. 19173389.8, filed on May 8, 2019.

### SEQUENCE LISTING

The instant application contains a Sequence Listing which has been submitted in ASCII format via EFS-Web and is hereby incorporated by reference in its entirety. Said ASCII copy, is named 028622-0321_SL.txt and is approximately 82 kb in size.

The present invention relates to new species-selective phage endolysins and their use to treat bacterial vaginosis (BV). The present invention provides recombinant endolysins, i.e. domain-swapped endolysins. The invention also relates to said endolysins for use in treating diseases or disorders such as bacterial infections, in particular BV. The invention further relates to polynucleotides encoding said endolysins. Said polynucleotides can also be used for treating such diseases or disorders. Also provided by the present invention is a pharmaceutical composition comprising an endolysin of the invention for use in treating such diseases or disorders. Said endolysins, polynucleotides and pharmaceutical composition may be administered locally, in particular locally into the vagina.

Bacterial vaginosis (BV), also been referred to in the literature as bacterial vaginitis, non-specific vaginosis and non-specific vaginitis, is the most common vaginal infection worldwide and is associated with significant adverse consequences including preterm labor and delivery, post-partum enodmetritis and an increased risk of HIV acquisition. It is a dysbiosis of the vagina where the commensal Lactobacilli are displaced by a polymicrobial biofilm, the pH increases from the natural 3.5-4.5 up to 5.5, and a malodorous fluid forms. Reported prevalence rates range from 10-40% depending upon the population studied. However, suboptimal methods of diagnosis and a high percentage of asymptomatic patients make the true prevalence of BV difficult to ascertain. Gardnerella vaginalis (G. vaginalis) is a bacterial species associated with BV.

The etiopathogenesis of BV remains poorly understood. It is most commonly defined as a pathological state characterized by the loss of normal vagina flora, particularly of H₂O₂-producing species of Lactobacillus, and the simultaneous overgrowth of anaerobic bacteria including G. vaginalis, Mobiluncus species, and Mycoplasma hominis. Recent data however, suggest a primary role for G. vaginalis as a specific and sexually transmitted etiological agent in BV (Muzny et al., 2016, J. of Infect. Dis. 214 Suppl. 1., S1).

In the 1950s, abundant small, pleomorphic gram-variable rods were observed in the genital tract of women with BV. This organism, first called Haemophilus vaginalis and repeatedly renamed as more information about its characteristics became available, is now classified as G. vaginalis which, until 2018, was considered to be the sole member of the genus Gardnerella. However in early 2019 it was shown that the genus Gardnerella actually contains at least 13 species, and the most frequent ones were renamed G. vaginalis sensu stricto, G. leopoldii, G. piotii, and G. swidsinskii (Vaneechoutte et al., 2019 Int. J. Syst. Evol. Biol. 898661).

Bacteria of the genus Gardnerella are special in that they are Gram-variable, i.e. they do not form the outer membrane defining the Gram-negative species. The cell wall is generally very thin and has only 10% or less content of peptidoglycan, which is why the crystal violet dye used for Gram staining does not always yield the deep purple color typical for Gram-positive species. Rather, Gardnerella cells can appear both Gram positive and negative in a Gram staining. Phylogenetic analysis based on 16S rRNA places Gardnerella in the gram-positive family Bifidobacteriales.

During BV, the epithelial surface is covered with a dense collection of G. vaginalis in a biofilm that is frequently recalcitrant to treatment. Biofilms are adherent communities of microorganisms held together by a polymeric matrix composed of polysaccharides, proteins and/or nucleic acids. The distinct gene expression pattern, as well as the physical structure of biofilms increases bacterial resistance to many negative stimuli including chemical disinfectants, pH extremes, host immune defenses and antibiotics. Standard of BV treatment are the antibiotics Metronidazole and Clindamycin, which however often fail to eradicate the biofilm, so that recurrence rates are up to 60% within 6 months. Furthermore, treatment with antibiotics wipes the vaginal microbiome, despite leaving some rests of viable biofilm, which opens this ecological niche for other pathogens, e.g. fungi. A frequent effect of BV treatments is therefore candidosis. Treatment of BV was also attempted with probiotics, specifically with beneficial Lactobacilli supposed to re-colonize the vagina. However, several clinical trials failed to show a benefit.

Therefore, there is a great need for new methods and compositions to treat G. vaginalis infections and particularly BV, e.g. by selectively killing bacterial cells of the genus Gardnerella, preferably without harming the beneficial Lactobacilli while they re-populate the vagina. Thus, the technical problem underlying the present invention is the provision of novel means and methods for the treatment of BV.

The technical problem is solved by provision of the embodiments characterized in the claims.

The present invention is based on the preparation of novel recombinant Gardnerella prophage endolysins with unexpected properties and structure which make them particularly suitable for various uses and methods, in particular for treating, decontaminating or detecting, bacterial infections and disorders, in particular in relation with Gardnerella.

A first aspect of the invention provides an endolysin comprising or consisting of

(i) a N-terminal catalytic domain, or a functional variant thereof;

(ii) a C-terminal cell-wall binding region, or a functional variant thereof, wherein the C-terminal cell-wall binding region comprises or consists of at least one cell-wall binding domain; and

(iii) a linker region between the N-terminal catalytic domain and the C-terminal cell-wall binding region,

wherein the endolysin has a killing activity against Gardnerella.

In one aspect of the invention the N-terminal catalytic domain is from a first natural endolysin, the linker region and the C-terminal cell-wall binding region are from a second natural endolysin, and the first and the second natural endolysins are encoded by different genomes from different prophages. Thus, the invention provides a recombinant endolysin comprising or consisting of

(i) a N-terminal catalytic domain, or a functional variant thereof;

(ii) a C-terminal cell-wall binding region, or a functional variant thereof, wherein the C-terminal cell-wall binding region comprises or consists of at least one cell-wall binding domain; and

(iii) a linker region between the N-terminal catalytic domain and the C-terminal cell-wall binding region,

wherein the N-terminal catalytic domain is from a first natural endolysin, the linker region and the C-terminal cell-wall binding region are from a second natural endolysin, and the first and the second natural endolysins are encoded by different genomes from different prophages, and

wherein said recombinant endolysin has a killing activity against Gardnerella.

Gardnerella is special in that it is a Gram-variable species: it does not form the outer membrane defining true Gram-negative species. Its cell wall is generally very thin and has only 10% or less content of peptidoglycan. This indicates that a peptidoglycan-degrading enzyme, such as endolysin proteins, could not efficiently lyse the bacterial cell walls of Gardnerella. However, in the context of the present invention novel recombinant endolysins have been identified which have the advantageous property that they effectively kill Gardnerella species, and thus, could be used as a novel therapy for the treatment of BV.

The healthy vagina is populated mainly by 3 species of Lactobacilli: L. crispatus, L. gasseri and L. jensenii. These maintain an acidic pH of 3.5-4.5, by producing lactic acid, and a protective oxidative milieu, by producing H₂O₂. Recovery from BV is associated with a re-population of the vagina with these Lactobacilli. However, antibiotics (which are conventionally used for the treatment of BV) have the disadvantages that they interfere with the process of re-population of the vagina with Lactobacilli. In contrast, the novel recombinant endolysins of the invention advantageously have a species-selective killing activity against Gardnerella and do not harm Lactobacilli. In addition, the appended Examples show that all tested Gardnerella strains have a low susceptibility to Metronidazole and Clindamycin, which are conventionally used in the treatment of BV. This could explain the high recurrence rates of BV. This also sustains that the endolysins of the invention are superior to antibiotics in the treatment of BV. Accordingly, treating BV with the endolysins of the invention is far advantageous to the currently available treatments, such as the treatments with the antibiotics Metronidazole and Clindamycin.

Herein the term “from a first natural endolysin” means that the respective part (i.e. the N-terminal catalytic domain) is identical to or a functional variant of a first natural endolysin. As defined herein, a functional variant is a polypeptide which has at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of the respective part (i.e. the N-terminal catalytic domain) of a first natural endolysin and results in a functional endolysin, wherein the function comprises killing activity against Gardnerella. The amino acid sequences of several natural endolysins are provided herein below and are summarized in Table 7.

In line with this term “from a second natural endolysin” means that the respective part (i.e. the linker region and C-terminal cell-wall binding region) is identical to or a functional variant of a second natural endolysin, i.e. an endolysin which is different from the first natural endolysin. As defined herein, a functional variant is a polypeptide which has at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of the respective part (i.e. the linker region and C-terminal cell-wall binding region) of the second natural endolysin and results in a functional endolysin, wherein the function comprises killing activity against Gardnerella.

Herein, the N-terminal catalytic domain is also referred to as “H-domain”. For example, the term “H2” refers to the H-domain of the natural endolysin (EL) 2. The “C-terminal cell-wall binding region” refers to one or more cell-wall binding domains. The linker and the cell-wall binding domains represent together the so-called “B-region”. For example, B10 refers to the B-region of the natural EL10. Likewise, B11_N refers to the N-terminal cell-wall binding domain of natural EL11, B12_C refers to the C-terminal cell-wall binding domain of natural EL12 and so on.

The invention further provides an endolysin comprising or consisting of

(i) a N-terminal catalytic domain consisting of a polypeptide comprising or consisting of the amino acid sequence of any one of SEQ ID NOs: 1 to 5, 7, or 10 to 12, or any functional variant thereof having at least 80% identity with the amino acid sequence of any one of SEQ ID NOs: 1 to 5, 7, or 10 to 12;

(ii) a C-terminal cell-wall binding region comprising or consisting of at least one cell-wall binding domain independently selected from the group consisting of polypeptides comprising or consisting of the amino acid sequence of any one of SEQ ID NOs: 15 to 24 and 26 to 33, respectively, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 15 to 24 and 26 to 33, respectively; and

(iii) a linker region between the N-terminal catalytic domain and the C-terminal cell-wall binding region,

wherein said endolysin has a killing activity against Gardnerella.

As shown in the appended Examples, the most active N-terminal catalytic domain (also referred to as “H-domain”) is H2 (SEQ ID NO: 2), followed by H7 (SEQ ID NO: 7), H10 (SEQ ID NO: 10) and H5 (SEQ ID NO: 5).

Thus, in a preferred aspect of the present invention the N-terminal catalytic domain is consisting of a polypeptide comprising or consisting of the amino acid sequence of any one of SEQ ID NOs: 2, 7, 10 and 5, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 2, 7, 10 and 5;

whereby the endolysin is functional, wherein the function comprises the ability to lyse the cell wall of Gardnerella.

Accordingly, in a preferred aspect of the present invention the N-terminal catalytic domain is consisting of a polypeptide which comprises or consists of the amino acid sequence of SEQ ID NO: 5, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 5; or more preferably comprising or consisting of the amino acid sequence of SEQ ID NO: 10, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 10; or even more preferably comprising or consisting of the amino acid sequence of SEQ ID NO: 7, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 7; or even more preferably comprising or consisting of the amino acid sequence of SEQ ID NO: 2, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 2;

whereby the endolysin is functional, wherein the function comprises the ability to lyse the cell wall of Gardnerella.

It is also shown in the appended Examples that of the B-regions B10 (comprising the cell-wall binding domains of SEQ ID NOs: 28 and 29) is the most active, followed by B11 (comprising the cell-wall binding domains of SEQ ID NOs: 30 and 31), B12 (comprising the cell-wall binding domains of SEQ ID NOs: 32 and 33), and B3 (comprising the cell-wall binding domains of SEQ ID NOs: 19 and 20).

Thus, in a preferred aspect of the present invention the cell-wall binding domain(s) of is/are selected from the group consisting of polypeptides comprising or consisting of the amino acid sequence of any one of SEQ ID NOs: 19, 20 and 28-33, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 19, 20 and 28-33;

whereby the endolysin is functional, wherein the function comprises the ability to lyse the cell wall of Gardnerella.

The endolysin of the present invention comprises preferably two cell-wall binding domains. In one aspect of the present invention the cell-wall binding domains (B-domains) of the endolysin of the invention consists of a polypeptide comprising or consisting of the amino acid sequence of any one of SEQ ID NOs: 19, 20 and 28-33, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 19, 20 and 28-33;

whereby the endolysin is functional, wherein the function comprises the ability to lyse the cell wall of Gardnerella.

In a preferred aspect of the present invention the endolysin comprises a first cell-wall binding domain and a second cell-wall binding domain, wherein said first cell-wall binding domain is selected from the group consisting of SEQ ID NOs: 15, 17, 19, 21, 23, 26, 28, 30 and 32, and said second cell-wall binding domain is selected from the group consisting of SEQ ID NOs: 16, 18, 20, 22, 24, 27, 29, 31 and 33. Preferably, said first cell-wall binding domain is N-terminally of said second cell-wall binding domain.

In a more preferred aspect of the present invention the endolysin comprises the two cell-wall binding domains (B-domains) of natural endolysin EL10 (SEQ ID NOs: 28 and 29), of natural endolysin EL11 (SEQ ID NOs: 30 and 31), of natural endolysin EL12 (SEQ ID NOs: 32 and 33), or of natural endolysin EL3 (SEQ ID NOs: 19 and 20), even more preferably of natural endolysin EL10 (SEQ ID NOs: 28 and 29); or a functional variant thereof. Said functional variant may also be a set of two B-domains having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequences of the two B-domains of natural endolysin EL10 (SEQ ID NOs: 28 and 29), of natural endolysin EL11 (SEQ ID NOs: 30 and 31), of natural endolysin EL12 (SEQ ID NOs: 32 and 33), or of natural endolysin EL3 (SEQ ID NOs: 19 and 20), even more preferably of natural endolysin EL10 (SEQ ID NOs: 28 and 29);

whereby the endolysin is functional, wherein the function comprises the ability to lyse the cell wall of Gardnerella.

In one aspect of the present invention the cell-wall binding domain(s) (B-domain(s)) comprise(s) or consist(s) of the amino acid sequence of SEQ ID NO: 19 and/or 20, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 19 and/or 20; more preferably comprises or consists of the amino acid sequence of SEQ ID NO: 32 and/or 33, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 32 and/or 33; even more preferably comprises or consists of the amino acid sequence of SEQ ID NO: 30 and/or 31, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 30 and/or 31; or even more preferably comprises or consists of the amino acid sequence of SEQ ID NO: 28 and/or 29, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 28 and/or 29;

whereby the endolysin is functional, wherein the function comprises the ability to lyse the cell wall of Gardnerella.

It is preferred that the sequence VNELL or VNKLL, more preferably VNELL, is located at the C-terminus of the B-domain. In case of the presence of a plurality of B-domains within the B-region, it is also preferred that the sequence VNELL or VNKLL, more preferably VNELL, is located at the C-terminus of each B-domain.

Surprisingly and unexpectedly, it has been found in the context of the present invention that several recombinant endolysins have a stronger activity than natural endolysins, especially when viewed across all 4 Gardnerella strains tested (i.e. Gardnerella vaginalis sensu strict, Gardnerella leopoldii, Gardnerella piotii and Gardnerella swidsinskii). Particularly H2B10, H2B11, H2B12 and H7B3 are each more active than all tested natural endolysins. Thus, recombinant endolysins according to the present invention exhibit significantly higher activity than the natural endolysins.

Therefore, it is preferred that the “killing activity against Gardnerella” of the recombinant endolysin of the invention is enhanced as compared to the killing activity of natural endolysins, e.g. natural endolysins EL1-EL12 (having the amino acid sequences as shown in Table 7).

In line with the considerably high activity of endolysins H2B10, H2B11, H2B12, and H7B3, these endolysins (and their functional variants) are preferred in the present invention. Thus, the endolysin of the present invention has preferably:

(i) a N-terminal catalytic domain consisting of a polypeptide comprising or consisting of the amino acid sequence of SEQ ID NO: 2 or 7, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 2;

(ii) a C-terminal cell-wall binding region comprising or consisting of at least one (preferably two) cell-wall binding domain(s) independently selected from the group consisting of polypeptides comprising or consisting of the amino acid sequence of any one of SEQ ID NOs: 19, 20 and 28 to 33, respectively, and any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 19, 20 and 28 to 33, respectively; and

(iii) a linker region between the N-terminal catalytic domain and the C-terminal cell-wall binding region consisting of a polypeptide comprising or consisting of the amino acid sequence X₁X₂GLNGX₃X₄NGGS, wherein X₁ is N or K, preferably N, X₂ is A, X₃ is Y and X₄ is K or Q,

wherein said endolysin has a killing activity against Gardnerella. Regarding the linker region it is indicated that, as mentioned below, the linker region may also consist of a polypeptide comprising or consisting of the amino acid sequence (XXX)n, wherein each X can be independently G, A or S, preferably wherein the amino acid sequence (XXX)n is (GGS)n, wherein n corresponds to the number of repetitions of the sequence XXX, preferably wherein n is 2, 3, 4, 5 or 6.

In the appended Examples the recombinant endolysin H2B10 was shown to have the highest activity. Therefore, it is most preferred in the present invention that the endolysin of the present invention is H2B10 (or a functional variant thereof). Accordingly, the endolysin of the present invention has most preferably:

(i) a N-terminal catalytic domain consisting of a polypeptide comprising or consisting of the amino acid sequence of SEQ ID NO: 2, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence SEQ ID NO: 2;

(ii) a C-terminal cell-wall binding region comprising or consisting of two cell-wall binding domains consisting of polypeptides comprising or consisting of the amino acid sequence of SEQ ID NO: 28 or 29, or any functional variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NO: 28 or 29; and

(iii) a linker region between the N-terminal catalytic domain and the C-terminal cell-wall binding region consisting of a polypeptide comprising or consisting of the amino acid sequence X₁X₂GLNGX₃X₄NGGS, wherein X₁ is N, X₂ is A, X₃ is Y and X₄ is K,

wherein said endolysin has a killing activity against Gardnerella. As mentioned above, it is preferred that the “killing activity against Gardnerella” of the recombinant endolysin of the invention is enhanced as compared to the killing activity of natural endolysins, e.g. natural endolysins EL1-EL12 (having the amino acid sequences as shown in Table 7).

Regarding the linker region it is indicated that, as mentioned below, the linker region may also consist of a polypeptide comprising or consisting of the amino acid sequence (XXX)n, wherein each X can be independently G, A or S, preferably wherein the amino acid sequence (XXX)n is (GGS)n, wherein n corresponds to the number of repetitions of the sequence XXX, preferably wherein n is 2, 3, 4, 5 or 6.

In the recombinant endolysin of the present invention the C-terminal cell-wall binding region may comprise or consists of one, two or three cell-wall binding domains. Said one, two or three cell-wall binding domains may be independently selected from the group consisting of the polypeptides comprising or consisting of the amino acid sequence of SEQ ID NOs: 15 to 24 and 26 to 33, respectively, and any variants thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NOs: 15 to 24 and 26 to 33, respectively, whereby said polypeptides are functional, wherein the function comprises the ability to bind to the cell wall of Gardnerella. It is preferred that the C-terminal cell-wall binding region consists of two cell-wall binding domains. Preferred C-terminal cell-wall binding regions are defined herein above and below.

The endolysin of the present invention does preferably not comprise the H-domain or B-region of natural endolysin EL6. The amino acid sequences of the H-domain and B-region of natural endolysin EL6 are shown in Table 7.

The linker region may consist of a polypeptide having a length of 6 to 18 amino acids, preferably a length of 9 to 15 amino acids, even more preferably a length of 12 amino acids. Preferably, the linker region may consist of a polypeptide comprising or consisting of the amino acid sequence (i) (XXX)n, wherein each X can be independently G, A or S, preferably wherein the amino acid sequence (XXX)n is (GGS)n, wherein n corresponds to the number of repetitions of the sequence XXX, preferably wherein n is 2, 3, 4, 5 or 6, or (ii) X₁X₂GLNGX₃X₄NGGS, wherein X₁ is N or K, X₂ is A or V, X₃ is Y or C and X₄ is K or Q. As described above, in one aspect of the endolysin of the present invention the N-terminal catalytic domain is identical to or derived from a first natural endolysin, the linker region and the C-terminal cell-wall binding region are identical to or derived from a second natural endolysin, and the first and the second natural endolysins are encoded by different genomes from different prophages.

The recombinant endolysin of the present invention has killing activity against Gardnerella. For example, the endolysin of the present invention may have killing activity against Gardnerella vaginalis sensu stricto, Gardnerella leopoldii, Gardnerella piotii and/or Gardnerella swidsinskii, preferably against all of them. The killing activity of the endolysins of the invention as described above against Gardnerella is preferably a genus-selective killing activity against Gardnerella. Herein “genus-selective killing activity against Gardnerella” means that the endolysin of the present invention does not have killing activity against bacteria in general. Preferably, the endolysin of the present invention has killing activity against Gardnerella, but not against Lactobacilli. In particular, it is preferred that said endolysin has no killing activity against Lactobacilli crispatus, Lactobacilli gasseri, and/or Lactobacilli jensenii. More preferably, said endolysin has no killing activity against all of these Lactobacilli, i.e. Lactobacilli crispatus, Lactobacilli gasseri, and Lactobacilli jensenii.

The invention also relates to a polynucleotide molecule encoding an endolysin as described above. The nucleic acid molecule may be DNA, e.g. cDNA, or RNA. Herein, the terms “polynucleotide” or “polynucleotide molecule” is used synonymously with the term “nucleic acid molecule” or the like.

The invention also relates to a vector comprising said polynucleotide molecule of the invention. In one embodiment, the vector is an expression vector. Any suitable vector known in the art may be used, such as the pET series of vectors and all the T7 based vectors. For example, the vector may be a plasmid. Thus, one aspect of the present invention relates to a plasmid comprising the polynucleotide of the invention. It will be appreciated by persons skilled in the art that the choice of expression vector may be determined by the choice of the host cell.

Also provided by the present invention is a host cell comprising the polynucleotide molecule according to the invention or the vector/plasmid according to the invention. In one embodiment, the host cell is a microbial cell, for example a bacterial cell. Preferably the host cell is non-pathogenic. Most preferably the host cell is E. coli. Thus, one aspect of the invention relates to a bacterial host cell comprising the plasmid of the invention, preferably wherein the bacterial host cell is an E. coli cell.

Also encompassed by the present invention is a method for producing the endolysin of the invention comprising culturing a population of host cells comprising the polynucleotide molecule according to the invention or a vector/plasmid according to the invention under conditions in which the endolysin is expressed, and isolating the endolysin therefrom.

A further aspect of the invention provides a pharmaceutical composition comprising

(a) an endolysin according to the invention;
(b) a polynucleotide molecule according to the invention;
(c) a vector/plasmid according to the invention;
(d) a host according to the invention; and/or
(e) a bacteriophage capable of expressing an endolysin according to the invention
and a pharmaceutically acceptable carrier, diluent or excipient. For example, the pharmaceutical composition of the present invention may comprise the endolysin of the invention, the polynucleotide molecule of the invention, and a pharmaceutically acceptable carrier and/or diluent.

A further aspect of the invention relates to

(a) an endolysin according to the invention;
(b) a polynucleotide molecule according to the invention;
(c) a vector/plasmid according to the invention;
(d) a host according to the invention;
(e) a bacteriophage capable of expressing an endolysin according to the invention; and/or
(f) a pharmaceutical composition according to the invention
for use in treating a disease or disorder. For example, the invention provides an endolysin according to the invention, a polynucleotide molecule according to the invention, or a pharmaceutical composition according to the invention for use in treating a disease or disorder. Said disease or disorder may be a bacterial infection, preferably bacterial vaginosis. For example, the bacterial vaginosis may be caused by Gardnerella vaginalis sensu strict, Gardnerella leopoldii, Gardnerella piotii and/or Gardnerella swidsinskii.

In one aspect of the present invention the recombinant endolysin of the invention, the polynucleotide molecule of the invention, or the pharmaceutical composition of the invention is to be administered locally, preferably locally into the vagina of a subject. Thus, in one aspect of the present invention the recombinant endolysin of the invention, the polynucleotide of the invention, or the pharmaceutical composition of the invention is to be administered into the vagina of a subject.

The appended Examples show that the activity of the recombinant endolysins of the present invention is particularly high at a pH around pH 5. Therefore, one aspect of the present invention relates to the recombinant endolysin of the invention, the polynucleotide molecule of the invention, or the pharmaceutical composition of the invention, wherein said recombinant endolysin, polynucleotide or pharmaceutical composition is to be co-administered with a compound or composition which adjusts the pH of the vagina to 4.0-6.0, preferably to 4.5-5.5, more preferably to about 5. Suitable compounds or compositions which adjusts the pH of the vagina include but are not limited to phosphate, lactic acid (e.g. the natural acidification substance which Lactobacilli secrete to establish an acidic milieu) or other organic acids, e.g. carboxy-substituted polymers.

A further aspect of the invention relates to

(a) an endolysin of the invention;
(b) a polynucleotide molecule of the invention;
(c) a vector/plasmid of the invention;
(d) a host of the invention;
(e) a bacteriophage capable of expressing an endolysin of the invention; and/or
(f) a pharmaceutical composition of the invention
for use as a medicament.

A further aspect of the invention concerns the use of

(a) an endolysin of the invention;
(b) a polynucleotide molecule of the invention;
(c) a vector/plasmid of the invention;
(d) a host of the invention;
(e) a bacteriophage capable of expressing a polypeptide of the invention; and/or
(f) a pharmaceutical composition of the invention
in the manufacture of a medicament for treating bacterial infections and disorders.

A further aspect of the invention provides a method for treating bacterial infections and disorders such as BV comprising administering a subject in need thereof, a therapeutically effective amount of

(a) an endolysin of the invention;
(b) a polynucleotide molecule of the invention;
(c) a vector/plasmid of the invention;
(d) a host of the invention;
(e) a bacteriophage capable of expressing a polypeptide of the invention; and/or
(f) a pharmaceutical composition of the invention.
In some embodiment, the therapeutically effective amount is a dose of 10 to 100 ug of endolysin, optionally to be administered several times per day.

A further aspect of the invention provides a kit comprising an endolysin as described herein and instructions of use, in particular for treating a disease or disorder, preferably BV as defined above. Said kit may also comprise a compound or composition which adjusts the pH of the vagina to 4.0-6.0, preferably to 4.5-5.5, more preferably to about 5. The definitions and preferred aspects defined herein above and below for the endolysin of the present invention apply, mutatis mutandis also for the polynucleotide molecule, vector/plasmid, host cell, pharmaceutical composition, method of treatment and kit of the present invention.

A further aspect of the invention provides an in vitro method for the diagnosis of a disease or condition which can be treated with the endolysin according to the present invention, the method comprising the steps of:

(i) contacting a sample obtained from the subject with a polypeptide comprising or consisting of the C-terminal cell-wall binding region of the endolysin according to the present invention, and optionally the N-terminal catalytic domain of the endolysin according to the present invention, wherein the sample comprises microbial cells, and wherein the C-terminal cell-wall binding region of said endolysin is optionally labelled;
(ii) testing whether the polypeptide binds to, and/or lyses, the microbial cells of the sample; and
(iii) determining that a disease or condition can be treated with the endolysin according to the present invention if the polypeptide binds to, and/or lyses, the microbial cells.
The microbial cells may be Gardnerella cells, preferably cells of G. vaginalis sensu stricto, G. leopoldii, G. piotii, G. swidsinskii or other species of the genus Gardnerella.
Other features and advantages of the invention will be apparent from the following detailed description.

### BRIEF DESCRIPTION OF THE DRAWINGS

FIG. 1 shows a sequence alignment of the natural Gardnerella prophage endolysins of the present disclosure (CLUSTAL O(1.2.4) multiple sequence alignment). The majority of the endolysins has 306 residues, except two which have 251 residues.

FIG. 2 shows a phylogenic tree of the natural Gardnerella prophage endolysins of the present disclosure. There are no identical pairs among the endolysins, even though they are highly homologous.

FIG. 3 shows a domain structure of the Gardnerella prophage endolysins of the present disclosure as determined with InterPro (Mitchell et al., 2019, Nucleic Acids Res. 47, D351-D360). The N-terminal part of 196 residues of the endolysins is identified as the catalytic domain, due to its homology to Glycoside hydrolases, family 25. The catalytic domain is followed by a linker region and two domains which are identified as two cell-binding domains, due to their homology to the C-terminal domain of lysozyme Cpl-7 (CW_7 domain). According to the nomenclature of the present application, the catalytic domain represents the hydrolase or “H-domain”, while the linker region and the cell-wall binding domains represent together the binding or “B-region”.

FIGS. 4A to 4C show three enzymatic activity assays where the enzymatic activity of natural Gardnerella prophage endolysins of the present disclosure is measured by detecting the change in turbidity of a suspension of Gardnerella cells. In FIG. 4A, the enzymatic activity of the endolysins is measured by detecting the change in turbidity of a suspension of the G. leopoldii strain Gv_10 at pH 6.0. In FIG. 4B, the enzymatic activity of the endolysins is measured by detecting the change in turbidity of a suspension of the G. piotii strain Gv_17 at pH 7.0. In FIG. 4C, the enzymatic activity of the endolysins is measured by detecting the change in turbidity of a suspension of the G. swidsinskii strain Gv_23 at pH 7.4. Treatment was conducted in a medium adjusted to the appropriate pH in a photometric cuvette against buffer. Then, the change in turbidity was assessed by measuring the optical density (OD) at 600 nm. As a result, the drop in turbidity was more pronounced for the endolysin treated groups than for the buffer, indicating enzymatic activity.

FIG. 5 shows a quantitative reduction in viable Colony Forming Units (CFU) assay upon treatment with Gardnerella prophage endolysins.

FIG. 6 shows a quantitative reduction in viable Colony Forming Units (CFU) assay comparing untreated cells from the G. vaginalis sensu stricto strain Gv_9 incubated in medium with or without imidazole at different pH values. 5×10⁷ CFU/ml cells were incubated under the conditions indicated below the graph for 5 hours at 37° C. under anaerobic conditions, after which the surviving CFU/ml was determined by quantitative plating. The results show that the survival of G. vaginalis Gv_9 is highly dependent on the absence of imidazole and on a low pH under the tested conditions.

FIG. 7 shows a quantitative reduction in viable Colony Forming Units (CFU) assay comparing cells from the G. vaginalis sensu stricto strain Gv_9 treated with an eluate solution containing recombinant endolysins H10B1 and 250 mM imidazole at different pH values or with a control containing 250 mM imidazole at different pH values. 5×10⁷ CFU/ml cells were incubated under the conditions indicated below the graph for 5 hours at 37° C. under anaerobic conditions, after which the surviving CFU/ml was determined by quantitative plating. The columns labeled imidazole control depict the same data as in FIG. 6. The results show that the enzymatic activity of H10B1, as an example for all the endolysins of the invention, is higher at low pH values, with pH 5.5 and pH 5.0 showing the strongest activity.

FIGS. 8A to 8D show four quantitative reduction in viable Colony Forming Units (CFU) assays measuring the killing activity of natural and recombinant Gardnerella prophage endolysins of the present disclosure against the four main species of Gardnerella. In FIGS. 8A, 8B, 8C and 8D, the killing activity of the endolysins is measured by detecting the viable CFU of suspensions of the G. vaginalis sensu stricto strain Gv_9, the G. leopoldii strain Gv_11, the G. piotii strain Gv_17, and the G. swidsinskii strain Gv_23, respectively. 90 ul 5e7 CFU/ml of the indicated strain were incubated for 5 hours at pH 5.0 under anaerobic conditions with 10 ul endolysin solution (concentration adjusted to 0.2 mg/ml where possible, see Table 4). The logarithmic Y axis depicts the count of surviving cells. The dotted line indicates the limit of detection (LOD) given by plating of 2 ul of the reaction mix (500 CFU/ml). The results show that the endolysins of the present invention have the capacity to lyse the four main species of Gardnerella. The results also point out that some of the recombinant endolysins of the invention have a higher killing activity than the natural endolysins of the invention.

FIG. 9 shows a phylogenetic relationship tree (amino acid level) of H-domains created with Clustal Omega (Sievers et al., 2011 Mol. Syst. Biol. 7, 539).

FIG. 10 shows a phylogenetic relationship tree (amino acid level) of B-regions created with Clustal Omega (Sievers et al., 2011 Mol. Syst. Biol. 7, 539).

FIG. 11 shows a sequence alignment of the cell-wall binding domains (also called B-domains) within the B-region of the natural endolysins of the invention with Clustal Omega (Sievers et al., 2011 Mol. Syst. Biol. 7, 539). For each B-region, the N-terminal cell-wall binding domain is denoted with a _N suffix (Bx_N) and the C-terminal cell-wall binding domain is denoted with a _C suffix (Bx_C). By way of example, B3_C designates the second (C-terminal) B-domains of B3.

FIG. 12 shows a phylogenetic relationship tree of the individual B-domains with Clustal Omega (Sievers et al., 2011 Mol. Syst. Biol. 7, 539).

FIG. 13 shows three quantitative reduction in viable Colony Forming Units (CFU) assays measuring the killing activity of recombinant Gardnerella prophage endolysins of the present disclosure against the three most frequent species of beneficial Lactobacilli, at pH 5.0 under anaerobic conditions. The results show that the endolysins of the invention are ineffective against the beneficial Lactobacilli strains.

FIG. 14 shows MIC microbroth dilution activity assays measuring the effect of Metronidazole and Clindamycin (obtained from Ratiopharm as a solution for injection, 300 mg/2 ml), on the growth in suspension of the four main species of Gardnerella. Gardnerella suspensions at 2.5×10⁷ CFU/ml were incubated with the concentration of antibiotics as indicated on the x-axis of each graph and incubated for 48 h at 37° C. under anaerobic conditions. Cell growth was evaluated by Optical Density measurement at 610 nm (OD(610)) before and after incubation to determine the Minimum Inhibitory Concentration (MIC). The results show that all Gardnerella strains are resistant both to Metronidazole and Clindamycin (obtained from Ratiopharm as a solution for injection, 300 mg/2 ml) exhibiting MICs of 64 to <128 μg/ml and 16 μg/ml, respectively (FIG. 14).

FIG. 15 shows MIC microbroth dilution activity assays measuring the effect of Metronidazole and Clindamycin hydrochloride (obtained from Sigma Aldrich), on Gardnerella suspensions at 1×10⁵-1×10⁶ CFU/ml. This time the results show that Metronidazole had a MIC on all tested Gardnerella strains between 8 and 128 μg/ml and Clindamycin hydrochloride powder (obtained from Sigma Aldrich (C5269-10MG)) exhibited MICs between 0.25 and 5 μg/ml.

FIG. 16 shows MIC microbroth dilution activity assays measuring the effect of H2B10, a representative of herein claimed domain swapped endolysins, on the growth of three main species of Gardnerella. Gardnerella suspensions at 1×10⁵-1×10⁶ CFU/ml were incubated with the concentration of H2B10 as indicated on the x-axis of each graph and incubated for 48 h at 37° C. under anaerobic conditions. Cell growth was evaluated by OD(610) measurements before and after the incubation to determine the Minimum Inhibitory Concentration (MIC). MIC values between 1 and 4 μg/ml were obtained indicating that all Gardnerella strains are highly sensitive to the domain swapped endolysin H2B10.

### DETAILED DESCRIPTION

### Definitions

The term “lysins” refers to cell-wall lytic enzymes encoded by bacteriophages (endolysins) or bacteria (autolysins) which have the ability to hydrolyze the cell-wall of target bacteria when added exogenously (lysis-from-without). This novel class of antibacterials has important advantages over classical antibiotics, e.g. a novel mode of action; a narrow spectrum of susceptible bacteria; rapid killing of both stationary- and exponentially-growing bacteria; activity on mucous membranes and bacterial biofilms; low probability of developing resistances; and reduced impact on normal microbiota. These unique features have boosted the interest on the biotechnological and pharmacological exploitation of lysins and their recent inclusion among the top current alternatives to fight antibiotic resistances. Lysins from Gram-positive bacteria and their phages usually comprise at least one catalytic domain and one or more cell wall-binding domains. In contrast, many lysins produced by Gram-negative species or their phages only contain the catalytic domain, though modular endolysins have also been reported. The catalytic units dictate the type of peptidoglycan (PG) bond to be cleaved, whereas the cell wall-binding domain(s) largely determines the lytic spectrum by specific recognition of cell wall elements distributed in genus-, or species/strain-specific manner.

In the context of the present disclosure, the term “natural endolysin” refers to an endolysin encoded by a prophage sequence within a bacterial genome, in particular within the genome of Gardnerella cells. In the context of the present disclosure, the term “natural endolysin” therefore refers to an endolysin which has not been domain-swapped. A natural endolysin can be unmodified, meaning that the amino acid sequence of the endolysin corresponds to the native sequence. Alternatively, a natural endolysin can be modified, meaning that the amino acid sequence of the endolysin comprises at least one mutation compared to the native sequence. The amino acid sequences of natural endolysins E1-E14 are shown in Table 7, below. An example of a known 1, 4-beta-N-acetylmuramidase (natural endolysin EL1) sequence is also provided under NCBI accession No. WP_014554482 (version WP_014554482.1 of May 27, 2013).

In the context of the present disclosure, the term “recombinant endolysin” refers to an endolysin which has been domain-swapped. In the context of the present disclosure, the term “domain-swapped endolysin” refer to an endolysin which possess a N-terminal catalytic domain from a first natural endolysin, and at least one cell-wall binding domain from a second natural endolysin, wherein the first and the second natural endolysin are encoded by different genomes from different prophages. A recombinant endolysin of the invention might comprise or consist of a N-terminal catalytic domain from a first natural endolysin, and two cell-wall binding domains from a second natural endolysin, wherein the first and the second natural endolysin are encoded by different genomes from different prophages. Alternatively, recombinant endolysin of the invention might comprise or consist of a N-terminal catalytic domain from a first natural endolysin, a first (N-terminal) cell-wall binding domain from a second natural endolysin, and a second (C-terminal) cell-wall binding domain from a third natural endolysin wherein the first and the second natural endolysin are encoded by different genomes from different prophages, and wherein the third natural endolysin is optionally encoded by a different genome from different a prophage than the first and the second natural endolysin. A recombinant endolysin can be unmodified, meaning that the amino acid sequence of the endolysin corresponds to the native sequence of the respective domains composing the endolysin. Alternatively, a recombinant endolysin can be modified, meaning that the amino acid sequence of the endolysin comprises at least one mutation compared to the native sequence of the respective domains composing the endolysin. In line with this definition, the person skilled in the art readily understands that the “domain-swapped” or “recombinant” endolysins as described herein are non-naturally occurring endolysins. That is, the recombinant endolysin of the present invention has been modified by hand of man and excludes, by definition, natural endolysins, i.e. as it can be naturally found in nature. The appended examples provide suitable method(s) how to generate the artificial endolysin of the invention.

The terms “catalytic domain” or “enzymatic domain” refer to the part of the protein chain which contains the region where the catalyzed chemical reaction takes place. In the context of the present disclosure the term “H-domain” refers to a part of an endolysin of the invention which contains a catalytic domain.

In the context of the present disclosure the term “B-region” refers to a part of an endolysin of the invention which comprises or consists of a polypeptide having a cell-wall binding activity. In a preferred embodiment, the B-region comprises or consists of a linker region and one, two or three cell-wall binding domains or “B-domains”.

In the context of the present invention the term “B-domain” refers to a cell-wall binding domain contained within the B-region.

In the context of the present disclosure the term “CW_7 domain” refers to a cell-wall binding domain of the protein Cpl-7, i.e. the endolysin encoded by the Streptococcus pneumoniae bacteriophage Cp-7, (see Bustamante et al., 2010 J. Biol. Chem. 285, 33184-33196, 2012 PLoS One 7, e46654). Briefly, the Cpl-7 protein has a C-terminal cell-wall binding region composed of 3 consecutive CW_7 domains. Each CW_7 domains is composed of a similar amino acid sequence of 38 amino acids long, called the “CW_7 motif” and defined by Interpro (Mitchell et al., 2019, Nucleic Acids Res. 47, D351-D360) as consisting of the amino acid sequence TVANEVIQGLWGNGQERYDSLANAGYDPQAVQDKVNEXL, wherein X is I in the CW_7 motifs No:1 (amino acids 207-245) and No:2 (amino acids 255-293) and wherein X is L in the CW_7 motif No:3 (amino acids 303-341). In the Cpl-7 protein, there are short, 9 residues linkers between the CW_7 motifs No:1 and No:2 and between the CW_motifs No:2 and No:3, so that the total repeat is 47 residues long. In comparison, the repeats of the natural endolysins of the present invention are 49 residues long.

The terms “Minimum Inhibitory Concentration” or “MIC” refer to the lowest concentration of a chemical, usually a drug, which prevents visible growth of bacterium. MIC was defined in the present application as the minimal concentration of antibiotic at which no growth was detectable after 48 h by OD measurement.

The terms “Minimum Bactericidal Concentration” or “MBC” refer to the lowest concentration of an antibacterial agent required to kill a particular bacterium. Usually, the MBC90 is measured, i.e. the antibiotic concentration killing 90% of cells within a defined time, while MBC has been defined in the present application as the minimal concentration fully eradicating a suspension of 2.5×10⁷ CFU/ml. While MIC is the lowest concentration of an antibacterial agent necessary to inhibit visible growth, MBC is the minimum concentration of an antibacterial agent that results in bacterial death of all cells in suspension.

The terms “peptide”, “polypeptide”, “protein” and variations of these terms refer to peptide, oligopeptide, oligomer or protein including fusion protein, respectively, comprising at least two amino acids joined to each other by a normal or modified peptide bond, such as in the cases of the isosteric peptides, for example. These terms also include herewith “peptidomimetics” which are defined as peptide analogs containing non-peptidic structural elements, which peptides are capable of mimicking or antagonizing the biological action(s) of a natural parent peptide. A peptidomimetic lacks classical peptide characteristics such as enzymatically scissile peptide bonds. A peptide or polypeptide can be composed of amino acids other than the 20 amino acids defined by the genetic code. It can be composed of L-amino acids and/or D-amino acids. A peptide or polypeptide can equally be composed of amino acids modified by natural processes, such as post-translational maturation processes or by chemical processes, which are well known to a person skilled in the art. Such modifications are fully detailed in the literature. These modifications can appear anywhere in the polypeptide: in the peptide skeleton, in the amino acid chain or even at the carboxy- or amino-terminal ends. A peptide or polypeptide can be branched following an ubiquitination or be cyclic with or without branching. This type of modification can be the result of natural or synthetic post-translational processes that are well known to a person skilled in the art. For example, peptide or polypeptide modifications can include acetylation, acylation, ADP-ribosylation, amidation, covalent fixation of a nucleotide or of a nucleotide derivative, covalent fixation of a lipid or of a lipidic derivative, the covalent fixation of a phosphatidylinositol, covalent or non-covalent cross-linking, cyclization, disulfide bond formation, demethylation, glycosylation including pegylation, hydroxylation, iodization, methylation, myristoylation, oxidation, proteolytic processes, phosphorylation, prenylation, racemization, seneloylation, sulfatation, amino acid addition such as arginylation or ubiquitination. Such modifications are fully detailed in the literature and well-known by the killed person of the art.

As used herewith “bacterial infections and disorders” refer to infections and disorders caused by bacteria, in particular infections and disorders caused by at least one strain of the Gardnerella genus selected from the group consisting of Gardnerella vaginalis sensu strict, Gardnerella leopoldii, Gardnerella piotii and Gardnerella swidsinskii, and other Gardnerella species. Bacterial infections and disorders include but are not limited to Bacterial Vaginosis (BV).

As defined herewith the terms “killing activity” of an endolysin against a particular bacteria represents a reduction in the number of viable bacteria cells caused by the lysing activity of said endolysin. The killing activity of the endolysin against said bacteria can be complete meaning that 100% of the bacterial cells have been lysed or partial meaning that at least about 80%, at least about 90%, at least about 95%, at least about 99%, or at least about 99.9% of the bacterial cells have been lysed. Killing activity can be determined by measuring a decrease in optical density at 610-620 nm of a bacterial cell suspension and/or a decrease in Colony Forming Units (CFU) per millilitre of a bacterial cell suspension after exposure to the endolysin to be tested.

As defined herewith the terms “binding capacity” of an endolysin to the cell wall of a particular bacteria refers to the ability of said endolysin to specifically interact and adhere to the cell wall of said bacteria. The binding capacity of an endolysin to the cell wall of a bacteria can be determined by methods know of the art.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or relieving the disease, i.e., causing regression of the disease and/or its symptoms or conditions such as improvement or remediation of damage. In particular, treatment of bacterial infections comprises preventing, decreasing or even eradicating the infection, for instance by killing the bacteria and, thus, controlling, reducing or inhibiting bacterial proliferation as well as reducing the number of viable bacterial cells. Herein it is preferred that the disease, e.g. BV is treated therapeutically in terms of a partial or complete cure of the disease or the symptoms.

The term “subject” as used herein refers to mammals. For examples, mammals contemplated by the present invention include human, primates, domesticated animals such as cattle, sheep, pigs, horses, laboratory rodents and the like. It is preferred that the subject is a human being.

The term “effective amount” as used herein refers to an amount of at least one endolysin according to the invention, composition or pharmaceutical formulation thereof, that elicits the biological or medicinal response in a tissue, system, animal or human that is being sought. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active polypeptide sufficient to reduce the progression of the disease, notably to reduce or inhibit the disorder or infection and thereby elicit the response being sought (i.e. an “inhibition effective amount”).

The term “efficacy” of a treatment according to the invention can be measured based on changes in the course of disease in response to a use or a method according to the invention. The efficacy of prevention of infectious disease is ultimately assessed by epidemiological studies in human populations, which often correlates with titers of neutralizing antibodies in sera, and induction of multifunctional pathogen specific T cell responses. Preclinical assessment can include resistance to infection after challenge with infectious pathogen. Treatment of an infectious disease can be measured by inhibition of the pathogen's growth or elimination of the pathogen (and, thus, absence of detection of the pathogen), correlating with pathogen specific antibodies and/or T cell immune responses.

The term “biological material” refers to any material or sample that is obtained from a subject's body. This includes, for instance, samples of whole blood, serum, plasma, urine, sputum, saliva, vaginal swabs, or spinal fluids.

The term “inanimate material or surface” includes solutions, medium, devices, objects, floor, surface of a table.

The term “medium” includes water, air or food.

The terms “pharmaceutical formulation” or “pharmaceutical composition” refer to preparations which are in such a form as to permit biological activity of the active ingredient(s) to be unequivocally effective and which contain no additional component which would be toxic to subjects to which the said formulation would be administered.

The term “pharmaceutically acceptable” refers to a carrier comprised of a material that is not biologically or otherwise undesirable.

The term “carrier” refers to any components present in a pharmaceutical formulation other than the active agent and thus includes diluents, binders, lubricants, disintegrants, fillers, coloring agents, wetting or emulsifying agents, pH buffering agents, preservatives and the like.

The term “variant” refers to a polypeptide including insertions, deletions, and/or substitutions, either non-conservative or preferably conservative, relative to the native amino acid sequence. For example, the polypeptide may comprise an amino acid sequence with at least 80% identity to the native amino acid sequence, preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity to said amino acid sequence. Percent identity can be determined by methods well known in the art, using suitable computer programs for example MatGAT 2.0 (Myers and Miller, CABIOS (1989) Preferably, % identity is identified over the whole lengths of the sequences to be compared. It will be appreciated that percent identity is calculated in relation to polypeptides whose sequence has been aligned optimally. Fragment and variants of an amino acid sequence may be made using any of the methods of protein engineering, directed evolution and/or site-directed mutagenesis well known in the art (for example, see Molecular Cloning: a Laboratory Manual, 3rd edition, Sambrook & Russell, 2001, Cold Spring Harbor Laboratory Press). It will be appreciated by skilled persons that a polypeptide according to the invention, or fragment, variant, or fusion thereof, may comprise or consist of a derivative of a native amino acid sequence, or a fragment or variant thereof. Chemical derivatives of one or more amino acids may be achieved by reaction with a functional side group. Such derivatised molecules include, for example, those molecules in which free amino acid groups have been derivatised to form amine hydrochlorides, p-toluene sulphonyl groups, carboxybenzoxy groups, f-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g., acetylation or thioglycolic acid amidation), terminal carboxylamidation (e.g., with ammonia or methylamine), and the like terminal modifications. It will be further appreciated by persons skilled in the art that peptidomimetic compounds may also be useful. Thus, by ‘polypeptide’ we include peptidomimetic compounds which exhibit endolysin activity. The term ‘peptidomimetic’ refers to a compound that mimics the conformation and desirable features of a particular polypeptide as a therapeutic agent.

### Endolysins According to the Invention

The endolysin of the present invention has an antibacterial activity against Gardnerella strains. The optimum pH at which the endolysin according to the invention exhibits an antibacterial activity is comprised between about 4 and 6, preferably a pH about 5. The endolysin of the present invention comprises or consists of

(i) a N-terminal catalytic domain, or a functional variant thereof;

(ii) a C-terminal cell-wall binding region, or a functional variant thereof, wherein the C-terminal cell-wall binding region comprises or consists of at least one cell-wall binding domain; and

(iii) a linker region between the N-terminal catalytic domain and the C-terminal cell-wall binding region,

and has a killing activity against Gardnerella cells.

In some embodiment, the N-terminal catalytic domain is from a first natural endolysin, the linker region and the C-terminal cell-wall binding region are from a second natural endolysin, and the first and the second natural endolysin are encoded by different genomes from different prophages. It is envisaged that the killing activity of the endolysins of the invention against Gardnerella is a species-selective killing activity against Gardnerella.

The N-terminal catalytic domain is a functional polypeptide, wherein the function comprises the ability to lyse the cell wall of Gardnerella. The N-terminal catalytic domain may be a N-acetylmuramidase, N-acetylmuramoyl-L-alanine amidases, L-alanoyl-D-glutamate endopeptidases, interpeptide bridge endopeptidases or N-acetyl-beta-D-glucosaminidases. Preferably, the N-terminal catalytic domain is a N-acetylmuramidase, most preferably a 1,4-beta-N-acetylmuramidase. For example, the N-terminal catalytic domain may be a polypeptide comprising or consisting of the amino acid of any one of SEQ ID NOs: 1 to 5, 7, or 10 to 12 or any variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 1 to 5, 7, or 10 to 12, whereby said polypeptide is functional, wherein the function comprises the ability to lyse the cell wall of Gardnerella. Preferably, the N-terminal catalytic domain is a polypeptide comprising the amino acid of SEQ ID NOs: 2 or 7 or any variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NOs: 2 or 7, whereby said polypeptide is functional, wherein the function comprises the ability to lyse the cell wall of Gardnerella.

According to the present invention the C-terminal cell-wall binding region is a functional polypeptide, wherein the function comprises the ability to bind to the cell wall of Gardnerella. The C-terminal cell-wall binding region may comprise or consist of one, two, three or more cell-wall binding domains. For example, the one, two, three or more cell-binding domains may be independently selected from the group consisting of the polypeptides comprising or consisting of the amino acid sequence of SEQ ID NOs: 15 to 24 and 26 to 33, respectively, and any variants thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of SEQ ID NOs: 15 to 24 and 26 to 33, respectively, whereby said polypeptides are functional, wherein the function comprises the ability to bind to the cell wall of Gardnerella. Preferably, the one, two, three or more cell-wall binding domains are independently selected from the group consisting of a polypeptide comprising the amino acid sequence of any one of SEQ ID NOs: 19, 20 and 28-33 or any variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 19, 20 and 28-33, whereby said polypeptide is functional, wherein the function comprises the ability to bind to the cell wall of Gardnerella. More preferably, the one, two, three or more cell-wall binding domains are selected independently selected from the group consisting of a polypeptide comprising the amino acid sequence of any one of SEQ ID NOs: 28-33 or any variant thereof having at least 80% identity (preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity) with the amino acid sequence of any one of SEQ ID NOs: 28-33, whereby said polypeptide is functional, wherein the function comprises the ability to bind to the cell wall of Gardnerella.

Most preferably, the C-terminal cell-wall binding region comprises a first cell-wall binding domain and a second cell-wall binding domain, wherein said first cell-wall binding domain is selected from the group consisting of SEQ ID NOs: 15, 17, 19, 21, 23, 26, 28, 30 and 32, and said second cell-wall binding domain is selected from the group consisting of SEQ ID NOs: 16, 18, 20, 22, 24, 27, 29, 31 and 33. In preferred embodiments, said first cell-wall binding domain is N-terminally of said second cell-wall binding domain.

In one aspect of the invention, the linker region consists of a polypeptide having a length of 6 to 18 amino acids, preferably a length of 9 to 15 amino acids, even more preferably a length of 12 amino acids. Preferably, the linker region consists of a polypeptide comprising or consisting of the amino acid sequence (i) (XXX)n, wherein each X can be independently G, A or S, preferably wherein the amino acid sequence (XXX)n is (GGS)n, wherein n corresponds to the number of repetitions of the sequence XXX, preferably wherein n is 2, 3, 4, 5 or 6, or (ii) X₁X₂GLNGX₃X₄NGGS, wherein X₁ is N or K, X₂ is A or V, X₃ is Y or C and X₄ is K or Q. The fragment comprising the linker may be absent. The fragment comprising the linker may also be present and may enhance the cell wall binding and/or lytic activity of the polypeptide of the invention.

The invention further provides an endolysin having a killing activity against Gardnerella as described above for use in treating a disease or disorder. The disease or disorder to be treated may be a bacterial infection, preferably bacterial vaginosis. The bacterial vaginosis may be caused by G. vaginalis sensu stricto, G. leopoldii, G. piotii, and/or G. swidsinskii, or other species of the genus Gardnerella.

The endolysin of the invention is preferably capable of binding specifically to and/or lysing cells of Gardnerella for use in a method of treating a Gardnerella infection such as BV.

As noted above, it is well established that many bacteriophage endolysins consist of two distinct domains (for example, see Sheehan et al., 1996, FEMS Microbiology Letters 140:23-28). One is a catalytic domain that is responsible for cell wall degradation and these are known to exist in several forms. The other domain is a cell-wall binding domain that recognizes a cell surface motif and permits attachment of the endolysins to that target cell. The precise pattern recognition involved in the latter is what provides the specificity. The enzymatic domain can be identified by its amino acid homology to other similar regions of lytic enzymes that share the same type of lytic activity. In the case of the natural Gardnerella prophage endolysins newly discovered by the inventors, the domain arrangement has been identified to consist of a N-terminal domain of 196 residues, followed by a linker region of 12 residues and two repeated domains of respectively 49 residues, except for EL6 and EL9 where there is only one incomplete domain of 43 residues. The native amino acid sequences of these newly discovered endolysins are summarized in Table 7. The inventors identified that the N-terminal domain is the catalytic domain due to its homology to Glycoside hydrolases, family 25 and that the two repeated domains are two cell-wall binding domains due to their homology to the C-terminal domain of lysozyme Cpl-7 (see Example 2 and FIG. 3).

In some embodiment, the fragment comprising the enzymatic domain is unmodified, i.e. corresponds to the native amino acid sequence. In an alternative embodiment, the fragment comprising the enzymatic domain may comprise alterations such as substitution, deletion, insertion of amino acids or any combination of alteration thereof. In some embodiment the fragment comprising the enzymatic domain is a variant fragment having at least 80%, preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, even more preferably at least 99.7% identity, and most preferably 100% identity with the amino acids sequences of any one of SEQ ID NOs: 1 to 5, 7, or 10 to 12.

In some embodiment, the fragment comprising the cell-wall binding domain is unmodified, i.e. corresponds to the native amino acid sequence. In an alternative embodiment, the fragment comprising the enzymatic domain may comprise alterations such as substitution, deletion, insertion of amino acids or any combination of alteration thereof. In some embodiment the fragment comprising the cell-wall binding domain is a variant fragment having at least 80%, preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, even more preferably at least 99.7% identity, and most preferably 100% identity with the amino acids sequences of any one of SEQ ID NOs: 15 to 24 and 26 to 33.

In a further aspect of the invention, the endolysin comprises or consists of a fusion of a polypeptide, or a fragment, variant, or derivative thereof. By “fusion” of a polypeptide we include a polypeptide which is fused to any other polypeptide. For example, the polypeptide may comprise one or more additional amino acids, inserted internally and/or at the N- and/or C-termini of the amino acid sequence of an endolysin according to the invention, or of a fragment, variant or derivative thereof.

Thus, as described above, in one embodiment the endolysin of the first aspect of the invention comprises a fragment consisting of one or more cell-wall binding domains comprising or consisting of the amino acid sequence of any one of SEQ ID NO: 15 to 24 and 26 to 33 (or a variant of such a domain sequence which retains the cell-wall binding activity thereof), respectively, to which is fused an enzymatic domain from a different source. Examples of other suitable enzymatic domains include but are not limited to L-alanoyl-D-glutamate endopeptidase, D-glutamyl-m-DAP endopeptidase, interpeptide bridge-specific endopeptidase, V-acetyl-ß-D-glucosaminidase (muramoylhydrolase), N-acetyl-ß-D-muramidase (lysozyme) or lytic transglycosylase. Also N-acetylmuramoyl-L-alanine amidase from other sources could be utilized.

In one aspect of the invention, the endolysin may be fused to a polypeptide or protein in order to facilitate purification of said endolysin. Examples of such fusions are well known to those skilled in the art. Similarly, the endolysin may be fused to an oligo-histidine tag such as His6 or to an epitope recognized by an antibody such as well-known Myc tag epitope. Fusions to any fragment variant or derivative of an endolysin according to the present invention are also included in the scope of the invention, It will be appreciated that fusions (or variants or derivatives thereof) which retain desirable properties, namely endolysin activity are preferred. It is also particularly preferred if the fusions are ones which are suitable for use in methods described herein. For example, the fusion may comprise a further portion which confers a desirable feature on the endolysin of the invention; for example, the portion may be useful in detecting or isolating the endolysin, promoting cellular uptake of the endolysin, or directing secretion of the protein from a cell. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the endolysin, as known to those skilled in the art.

An essential feature of the endolysins of the invention is the ability to lyse cells of Gardnerella genus. Preferably, the endolysin is capable of lysing cells of multiple strains of Gardnerella. Most preferably, the endolysin is capable of lysing all strains of the genus Gardnerella, including G. vaginalis sensu stricto, G. leopoldii, G. piotii and G. swidsinskii (Vaneechoutte et al., 2019 Int. J. Syst. Evol. Biol. 898661). In one embodiment, the endolysins of the invention are substantially or completely incapable of lysing bacteria which are commensal members of the microbiota of a healthy vagina (and not known to cause adverse effects on the host). For example, it is advantageous if the endolysins do not lyse cells of Lactobacilli genus. Most preferably, the endolysins of the invention are substantially or completely incapable of lysing cells of L. crispatus, L. gasseri and L. jensenii. Optionally, the endolysins of the invention do not lyse cells of L. iners. Advantageously, the endolysin is capable of lysing cells of pathogenic bacteria selectively, i.e. to a greater extent than cells of non-pathogenic bacteria.

The killing activity of an endolysin according to the invention on a particular microorganism may be determined by standard procedures in the field including those based on the determination of the Minimum Inhibitory Concentrations (MICs) of an antimicrobial agent defined as the lowest concentration of said antimicrobial agent that inhibits the visible growth of a microorganism after overnight incubation as described in Andrews, 2001, J Antimicrobial Chemotherapy, 48, Suppl. SI, 5-16 or in “Document M7-A7, Methods for dilution antimicrobial susceptibility tests for bacteria that grow aerobically; Approved standards, 7th Edition, January 2006, vol. 26, No. 2” published by Clinical and Laboratory Standards Institute. Another suitable method for determining the killing activity of an endolysin according to the invention is described in the example section of the present application and consists in determining the decrease of the Optical Density measured at 610-620 nm of a suspension of the bacteria the susceptibility of which is to be tested in an in vitro turbidity assay performed in presence of purified endolysin according to the invention. According to another embodiment, in an in vitro turbidity test as described herewith, an endolysin according to the invention decreases the OD(610-620 nm) of a suspension of at least one strain of Gardnerella bacteria by more than 20%, more than 30%, more than 40%, more than 50%, more than 60%, more than 70%, more than 80%, more than 90%, or more than 95%.

Methods for the production of endolysins, or a fragment, variant, fusion, or derivative thereof, for use according to the invention are well known in the art. Conveniently, the endolysin, or fragment, variant, fusion or derivative thereof, is or comprises a recombinant endolysin. The endolysin according to the invention can be produced by standard techniques of genetic engineering comprising the use of a recombinant vector comprising a polynucleotide encoding an endolysin as described herewith. Numerous expression systems can be used including bacterial plasmids and derived vectors, transposons, yeast episomes, insertion elements, yeast chromosome elements, viruses such as baculovirus, papilloma viruses such as SV40, vaccinia viruses, adenoviruses, fox pox viruses, pseudorabies viruses, retroviruses, cosmid or phagemid derivatives. The nucleotide sequence can be inserted in the recombinant expression vector by methods well known to a person skilled in the art such as, for example, those that are described in MOLECULAR CLONING: A LABORATORY MANUAL, Sambrook et al., 4th Ed., Cold Spring Harbor Laboratory Press, Cold Spring Harbor, N.Y., 2001. The recombinant vector can include nucleotide sequences that control the regulation, the expression, the transcription, and/or the translation of the polynucleotide encoding the endolysin, these sequences being selected according to the host cells that are used. The recombinant vector can further include nucleotide sequences such as those encoding His tags for facilitating the purification step. Subsequently, such a recombinant vector is introduced in a host cell according to methods that are well known to a person skilled in the art, such as those described in BASIC METHODS IN MOLECULAR BIOLOGY, Davis et al., 2nd ed., McGraw-Hill Professional Publishing, 1995, and MOLECULAR CLONING: A LABORATORY MANUAL, supra, such as transfection by calcium phosphate, transfection by DEAE dextran, transfection, microinjection, transfection by cationic lipids, electroporation, transduction or infection. The host cell can be, for example, bacterial cells such as E. coli, cells of fungi such as yeast cells and cells of Aspergillus, Streptomyces, insect cells, Chinese Hamster Ovary cells (CHO), C127 mouse cell line, BHK cell line of Syrian hamster cells, Human Embryonic Kidney 293 (HEK 293) cells. Preferably, the host cell is E. coli. Said host cells are then cultivated in appropriate conditions so as to produce the endolysin described herewith, which can then further be purified from the culture medium or from the host cell lysate by any standard purification methods including, Immobilized-Metal Affinity Chromatography (IMAC) (Block et al. 2008, Protein Expr. Purif 27, 244-254).

### Compositions According to the Invention

In a further aspect of the invention are provided antibacterial compositions comprising an endolysin according to the first aspect of the invention, a nucleic acid according to the second aspect of the invention, a vector/plasmid according to the third aspect of the invention, a host cell according to the fourth aspect of the invention or a bacteriophage capable of expressing an endolysin according to the first aspect of the invention, in particular pharmaceutical compositions.

As used herein, “pharmaceutical composition” means a therapeutically effective formulation for use in the methods of the invention. A “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce, and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. In one embodiment of the invention, the pharmaceutical composition comprises an endolysin according to the first aspect of the invention. Thus, the pharmaceutical formulation may comprise an amount of an endolysin, or fragment, variant, fusion or derivative thereof, sufficient to inhibit at least in part the growth of cells of the genus Gardnerella in a patient who is infected or susceptible to infection with such cells. Preferably, the pharmaceutical formulation comprises an amount of endolysin, or fragment, variant, fusion or derivative thereof, sufficient to kill cells of the genus Gardnerella in the patient. It will be appreciated by persons skilled in the art that the endolysins of the invention are generally administered in admixture with a suitable pharmaceutical excipient, diluent or carrier selected with regard to the intended route of administration and standard pharmaceutical practice (for example, see Remington: The Science and Practice of Pharmacy, 19^{th} edition, 1995, Ed. Alfonso Gennaro, Mack Publishing Company, Pennsylvania, USA). For example, the endolysins can be administered locally, i.e. locally into the vagina of a female subject and/or, in a male subject into or on the glans penis, prepuce or urethral entry. Herein the term “(administration) into or on the glans penis” also includes “(administration) into and on the glans penis”. In line with this, the term “(administration) into or on the glans penis, prepuce or urethral entry of a male subject” also includes “(administration) into and on the glans penis and on the prepuce and on the urethral entry of a male subject”. In another embodiment, the endolysins can be co-administered with a compound or composition which adjusts the pH of the vagina. In some embodiment the compound or composition adjusts the pH of the vagina to pH 4.0 to 6.0, preferably to pH 5.0.

In an alternative embodiment of the invention, the pharmaceutical compositions do not comprise the endolysin itself but instead comprise a nucleic acid molecule capable of expressing said endolysin. Suitable nucleic acid molecules, expression vectors, and host cells are described in detail above. For example, a recombinant probiotic may be used (LAB strain, e.g., Lactococcus lactis or a Lactobacillus sp.). In a further embodiment of the invention, the pharmaceutical compositions comprise a bacteriophage capable of expressing an endolysin according to the first aspect of the invention. Methods for performing such bacteriophage-based therapies are well known in the art (for example, see Watanabe et al., 2007, Antimicrobial Agents & Chemotherapy 51:446-452). Thus, for treatment of bacterial infections described herein, the endolysin of the invention may be administered as the cognate protein, as a nucleic acid construct, vector or host cell which expresses the cognate protein, as part of a living organism which expresses the cognate protein (including bacteriophages), or by any other convenient method known in the art so as to achieve contact of the endolysin with its bacterial target, whether that be a pathogenic bacterium, such as G. vaginalis, or another pathogen or potential pathogen, as further described herein.

Compositions of the invention can contain one or more endolysin polypeptides. In this embodiment, endolysin polypeptides can either be present as independent polypeptides or as fusion proteins comprising said endolysin polypeptides or fragments thereof.

Pharmaceutical compositions of this invention may further comprise one or more pharmaceutically acceptable additional ingredient(s) such as alum, stabilizers, antimicrobial agents, buffers, coloring agents, flavoring agents, adjuvants, and the like. It is preferred that the pharmaceutical composition of the invention does not comprise imidazole.

The endolysins of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, aerosols, emulsions, elixirs, or capsules filled with the same, all for oral use, or in the form of sterile injectable solutions for parenteral (including subcutaneous) use. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed. Compositions of this invention may also be liquid formulations including, but not limited to, aqueous or oily suspensions, solutions, emulsions, syrups, and elixirs. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid. Further materials as well as processing techniques and the like are set out in Part 5 of Part 5 of Remington's “The Science and Practice of Pharmacy”, 22nd Edition, 2012, University of the Sciences in Philadelphia, Lippincott Williams & Wilkins.

Solid compositions of this invention may be in the form of tablets or lozenges formulated in a conventional manner. Tablets may be coated according to methods well known in the art. Injectable compositions are typically based upon injectable sterile saline or phosphate-buffered saline or other injectable carriers known in the art.

Compositions of this invention may also be formulated as suppositories, which may contain suppository bases including, but not limited to, cocoa butter or glycerides. Compositions of this invention may also be formulated transdermal formulations comprising aqueous or non-aqueous vehicles including, but not limited to, creams, ointments, lotions, pastes, medicated plaster, patch, or membrane.

Compositions of this invention may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions of this invention may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

The compounds of this invention can also be administered in sustained release forms or from sustained release drug delivery systems. A description of representative sustained release materials can also be found in Remington's “The Science and Practice of Pharmacy”.

### Method of Administration

Compositions of this invention are preferably administered locally into the vagina of a female subject and/or into or on the glans penis, prepuce or urethral entry of a male subject. However, these compositions may also be administered in any manner including intravenous injection, intra-arterial, intraperitoneal injection, subcutaneous injection, intramuscular, intra-thecal, oral route including sublingually or via buccal administration, topically, cutaneous application, direct tissue perfusion during surgery or combinations thereof.

In a preferred embodiment the endolysins, polynucleotides or pharmaceutical compositions of the present invention as described herein are to be administered locally. In a further preferred embodiment the endolysins, polynucleotides or pharmaceutical compositions of the present invention as described herein are to be administered into the vagina of a female subject and/or into or on the glans penis, prepuce or urethral entry of a male subject. In a further preferred embodiment the endolysins, polynucleotides or pharmaceutical compositions of the present invention as described herein are to be administered locally into the vagina of a subject.

The dosage administered, as single or multiple doses, to an individual will vary depending upon a variety of factors, including pharmacokinetic properties, patient conditions and characteristics (sex, age, body weight, health, size), extent of symptoms, concurrent treatments, frequency of treatment and the effect desired.

According to one aspect, the compositions of the invention may be administered in a preventive manner to patients before sexual relations.

### Combination

According to the invention, an endolysin can be administered alone or in combination with a co-agent useful in the prevention and/or treatment of Gardnerella infections or disorders, including those caused by Gardnerella vaginalis sensu stricto, Gardnerella leopoldii, Gardnerella piotii, Gardnerella swidsinskii and/or other species of the genus Gardnerella.

An endolysin according to the invention can be administered in combination with

(a) one or more conventional antibiotic treatments. Such antibiotics may include Clindamycin, Metronidazole or any other suitable antibiotics known by a skilled person in the art;
(b) one or more additional endolysins, or nucleic acid molecules, vectors, host cell or bacteriophage capable of expressing the same;
(c) a compound or composition adjusting the pH of the vagina. In some embodiment the compound or composition adjusts the pH of the vagina to pH 4.0 to 6.0, preferably to pH 5.0. Suitable pH adjusting compounds may include phosphate, lactic acid (e.g. the natural acidification substance which Lactobacilli secrete to establish an acidic milieu) or other organic acids, e.g. carboxy-substituted polymers;
(d) a therapy to neutralize the toxins released upon bacterial lysis of Gardnerella cells within the vagina. Suitable neutralising therapies may include antibodies (see Babcock et al., 2006, Infect. Immun. 74:6339-6347) and toxin absorbing agents such as tolevamer (see Barker et al., 2006, Aliment. Pharmacol. Ther. 24:1525-1534);
(e) a probiotic.

### Uses and Methods According to the Invention

A further aspect of the invention provides an endolysin according to the invention, a nucleic acid according to the invention, a vector according to the invention, a host cell according to the invention, a bacteriophage capable of expressing an endolysin according to the invention, or a pharmacological composition according to the invention for use in medicine. Hence, the endolysins of the invention may be for use in a method for treatment of the human or animal body by surgery or therapy and/or diagnostic methods practiced on the human or animal body. In particular, the invention provides an endolysin according to the invention, a nucleic acid of the invention, a vector/plasmid of the invention, a host cell of the invention, a bacteriophage capable of expressing an endolysin of the invention, or a pharmacological composition of the invention for use in treating a disease or disorder.

A further aspect of the invention provides an endolysin of the invention, a nucleic acid of the invention, a vector/plasmid of the invention, a host cell of the invention, a bacteriophage capable of expressing an endolysin of the invention, or a pharmacological composition according to the invention for use as a medicament.

An further aspect of the invention provides the use of a endolysin of the invention, a nucleic acid of the invention, a vector/plasmid of the invention, a host cell of the invention, a bacteriophage capable of expressing an endolysin of the invention, or a pharmacological composition of the invention, in the preparation of a medicament for killing and/or inhibiting/preventing the growth of microbial cells in a patient, wherein the microbial cells are selected from the group consisting of Gardnerella cells and other bacterial cells susceptible to lysis with said endolysin. In particular, the invention provides the use of a endolysin of the invention, a nucleic acid of the invention, a vector/plasmid of the invention, a host cell of the invention, a bacteriophage capable of expressing an endolysin of the invention, or pharmacological composition of the invention, in the manufacture of a medicament for treating bacterial infections and disorders.

A further aspect of the invention provides an endolysin of the invention, a nucleic acid of the invention, a vector/plasmid of the invention, a host cell of the invention or a pharmacological composition of the invention for use in killing and/or inhibiting/preventing the growth of microbial cells in a patient, wherein the microbial cells are selected from the group consisting of Gardnerella cells and other bacterial cells susceptible to lysis with said endolysin.

A further aspect of the invention provides a method for killing and/or inhibiting/preventing the growth of microbial cells in a patient the method comprising administering to the patient an endolysin of the invention, a nucleic acid of the invention, a vector/plasmid of the invention, a host cell of the invention, a bacteriophage capable of expressing an endolysin of the invention or pharmacological composition of the invention, wherein the microbial cells are selected from the group consisting of Gardnerella cells and other bacterial cells susceptible to lysis with said endolysin.

An further aspect of the invention provides the use of an endolysin of the invention, a nucleic acid of the invention, a vector/plasmid of the invention, a host cell of the invention, a bacteriophage capable of expressing an endolysin of the invention or a pharmacological composition of the invention in the preparation of a medicament for the treatment or prevention of a disease or condition associated with microbial cells in a patient, wherein the microbial cells are selected from the group consisting of Gardnerella cells and other bacterial cells susceptible to lysis with said endolysin.

A further aspect of the invention provides an endolysin of the invention, a nucleic acid of the invention, a vector/plasmid of the invention, a host cell of the invention, a bacteriophage capable of expressing an endolysin of the invention or a pharmacological composition of the invention for use in the treatment or prevention of a disease or condition associated with microbial cells in a patient, wherein the microbial cells are selected from the group consisting of Gardnerella cells and other bacterial cells susceptible to lysis with the endolysins of the invention.

A further aspect of the invention provides a method for the treatment or prevention of a disease or condition associated with microbial cells in a patient in need of such treatment, the method comprising administering to the patient an endolysin of the invention, a nucleic acid of the invention, a vector/plasmid of the invention, a host cell of the invention, a bacteriophage capable of expressing an endolysin of the invention or a pharmacological composition of the invention, wherein the microbial cells are selected from the group consisting of Gardnerella cells and other bacterial cells susceptible to lysis with the endolysins of the invention.

“A disease or condition associated with microbial cells in a patient” includes diseases and conditions arising from or antagonised by infection of a patient with Gardnerella. Such diseases and conditions include BV.

By ‘treatment’ we include both therapeutic and prophylactic treatment of a subject (or patient). In one embodiment, the endolysin of the invention, nucleic acid of the invention, vector/plasmid of the invention, host cell of the invention, bacteriophage capable of expressing an endolysin of the invention or the pharmacological composition of the invention, uses and methods of the invention are for the treatment of an existing disease or condition. Alternatively or additionally, the uses and methods of the invention may be for prophylaxis. The term ‘prophylactic’ or ‘prophylaxis’ is used to encompass the use of an endolysin or composition described herein which either prevents or reduces the likelihood of infection with Gardnerella in a patient or subject. The prophylaxis may be primary prophylaxis (i.e., to prevent the development of a disease) or secondary prophylaxis (where the disease has already developed and the patient is protected against worsening of this process). It is preferred that the means and methods provided herein are for the treatment of an existing disease or condition, particularly for the treatment of an existing BV.

As discussed above, the term ‘effective amount’ is used herein to describe concentrations or amounts of endolysins according to the present invention which may be used to produce a favourable change in a disease or condition treated, whether that change is a remission, a favourable physiological result, a reversal or attenuation of a disease state or condition treated, the prevention or the reduction in the likelihood of a condition or disease state occurring, depending upon the disease or condition treated. In one embodiment, the endolysin according to the first aspect of the invention, nucleic acid according to the second aspect of the invention, vector according to the third aspect of the invention, host cell according to the fourth aspect of the invention, bacteriophage capable of expressing an endolysin according to the first aspect of the invention or pharmacological composition according to the sixth aspect of the invention is administered in a single dose. Alternatively, the endolysin, nucleic acid, vector/plasmid, host cell, bacteriophage or pharmacological composition is administered as a plurality of doses (for example, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses). The endolysin, nucleic acid, vector/plasmid, host cell, bacteriophage or pharmacological composition is preferably administered at a frequency sufficient to maintain a continuous presence of the endolysin according to the first aspect of the invention in the vagina of the subject. Preferably, the dose and dosage frequency is sufficient to prevent occurrence or recurrence of a disease or condition associated with microbial cells in a subject (e.g., Gardnerella). Preferably, the dose and dosage frequency is sufficient to prevent occurrence or recurrence of growth impedance associated with microbial cells in a subject (e.g., Gardnerella).

In one embodiment, the uses and methods of the invention a host cell or pharmacological composition comprising a host cell is used to deliver the endolysin of the first aspect of the invention (preferably a host cell).

It will be appreciated that the medicaments described herein may be administered to a subject in combination with one or more additional therapeutic agents. For example, the medicaments described herein may be administered to a subject in combination with:

(a) one or more conventional antibiotic treatments. Such antibiotics may include Clindamycin, Metronidazole or any other suitable antibiotics known by a skilled person in the art
(b) one or more additional endolysins, or nucleic acid molecules, vectors, host cell or bacteriophage capable of expressing the same;
(c) a compound or composition which adjusts the pH of the vagina, preferably to pH 4.0 to 6.0, more preferably to about pH 5.0. Such pH adjusting compounds may include phosphate, lactic acid (e.g. the natural acidification substance which Lactobacilli secrete to establish an acidic milieu) or other organic acids, e.g. carboxy-substituted polymers;
(d) a therapy to neutralize the toxins released upon bacterial lysis of G. vaginalis cells within the vagina. Suitable neutralising therapies may include antibodies (see Babcock et al., 2006, Infect. Immun. 74:6339-6347) and toxin absorbing agents such as tolevamer (see Barker et al., 2006, Aliment. Pharmacol. Ther. 24:1525-1534)
(e) a probiotic.

A further aspect of the invention provides the use of an endolysin having a cell lysing activity against Gardnerella, or a nucleic acid molecule, vector/plasmid, host cell or bacteriophage capable of expressing the same, for killing and/or inhibiting/preventing the growth of microbial cells in vitro and/or ex vivo, wherein the microbial cells are selected from the group consisting of Gardnerella cells and other bacterial cells susceptible to lysis with the endolysins of the invention. For example, the endolysins having said activity may be used to clean surfaces, such as those in hospitals, kitchens, etc, which may be susceptible to contamination with such bacterial cells. Preferably, the microbial cells comprise or consist of cells of G. vaginalis sensu stricto, G. leopoldii, G. piotii, G. swidsinskii, or other species of the genus Gardnerella.

A further aspect of the present invention provides a kit. Said kit comprises an endolysin as described herein and instructions of use, in particular for treating a disease or disorder, preferably BV. Said kit may be used for therapeutic or prophylactic purposes and may further comprise a compound or composition which adjusts the pH of the vagina to 4.0-6.0, preferably to 4.5-5.5, more preferably to about 5. However, the kit of the present invention may also be used for detecting the presence of microbial cells in a sample, the kit comprising a polypeptide having the cell lysing activity and/or cell binding specificity of an endolysin according to the invention or a nucleic acid molecule, vector/plasmid, host cell or bacteriophage capable of expressing the same, wherein the microbial cells are selected from the group consisting of Gardnerella cells and other bacterial cells susceptible to lysis with said endolysin.

Related aspects of the invention provide:

(a) the use of a polypeptide having the cell wall binding activity and/or cell lysing activity of an endolysin according to the invention or a nucleic acid molecule, vector/plasmid, host cell or bacteriophage capable of expressing the same, in the preparation of a diagnostic agent for a disease or condition associated with microbial cells selected from the group consisting of Gardnerella cells and other bacterial cells susceptible to lysis with said endolysin;
(b) the use of a polypeptide having the cell wall binding activity and/or cell lysing activity of an endolysin according to the invention or a nucleic acid molecule, vector/plasmid, host cell or bacteriophage capable of expressing the same, for the diagnosis of a disease or condition associated with microbial cells selected from the group consisting of Gardnerella cells and other bacterial cells susceptible to lysis with said endolysin;
(c) the use of a polypeptide having the cell wall binding activity and/or cell lysing activity of an endolysin according to the invention or a nucleic acid molecule, vector/plasmid, host cell or prophage capable of expressing the same, for detecting the presence of microbial cells in a sample in vitro and/or ex vivo, wherein the microbial cells selected from the group consisting of Gardnerella cells and other bacterial cells susceptible to lysis with said endolysin; and
(d) an in vitro method for the diagnosis of a disease or condition which can be treated with the endolysin according to the present invention, the method comprising the steps of: (i) contacting a sample obtained from the subject with a polypeptide comprising or consisting of the C-terminal cell-wall binding region of the endolysin according to the present invention, and optionally the N-terminal catalytic domain of the endolysin according to the present invention, wherein the sample comprises microbial cells, and wherein the C-terminal cell-wall binding region of said endolysin is optionally labelled; (ii) testing whether the polypeptide binds to, and/or lyses, the microbial cells of the sample; and (iii) determining that a disease or condition can be treated with the endolysin according to the present invention if the polypeptide binds to, and/or lyses, the microbial cells. The microbial cells may be Gardnerella cells, preferably cells of G. vaginalis sensu stricto, G. leopoldii, G. piotii, G. swidsinskii or other species of the genus Gardnerella.
Thus, the invention provides an in vitro method for the diagnosis of a disease or condition which can be treated with the endolysin of the invention in a subject, the method comprising contacting a cell sample obtained from the subject with a polypeptide having the cell wall binding activity and/or cell lysing activity of an endolysin according to the invention, or a nucleic acid molecule, vector/plasmid, host cell or prophage capable of expressing the same, and determining whether the cells in the sample have been lysed thereby, wherein the microbial cells are selected from the group consisting of Gardnerella cells and other bacterial cells susceptible to lysis with said endolysin. Preferably, the microbial cells comprise or consist of cells of G. vaginalis sensu stricto, G. leopoldii, G. piotii, G. swidsinskii or other species of the genus Gardnerella. In such diagnostic uses and methods, lysis of the cells may be detected using methods well known in the art. For example, levels of ATP may be measured as an indicator of cell lysis.

In an alternative embodiment of the above defined uses and methods of the invention, the polypeptide comprises or consists of the cell wall binding domain of an endolysin according to the invention. To permit detection, such a polypeptide may be fused to magnetic beads or used as a fusion protein comprising a suitable reporter or label (for example, green fluorescent protein or a color forming enzyme like HRP). Such diagnostic approaches are well established for endolysins from other systems, such as Listeria endolysins (for example, see Loessner et al., 2002, Mol Microbiol 44, 335-49; Kretzer et al, 2007, Applied Environ. Microbiol. 73:1992-2000).

Illustrative embodiments of the invention are described in the following non-limiting examples, with reference to the following figures.

### EXAMPLES

### Example 1—Identification of Natural Endolysins in Gardnerella Genomes

Endolysins are hydrolytic enzymes produced by bacteriophages in order to cleave the host's cell wall during the final stage of the lytic cycle. They have the capacity of targeting one of the five bonds in peptidoglycan (murein), the main component of bacterial cell walls, which allows the release of progeny virions from the lysed cell. To date, no bacteriophages lytic against Gardnerella have been isolated. Therefore it was also unknown whether endolysins from bacteriophage origins and having a lytic activity against Gardnerella could be successfully identified. The inventors investigated whether endolysins encoded by prophage sequences can be identified on various Gardnerella genomes. Prophages are bacteriophage genomes inserted and integrated into the circular bacterial DNA chromosome or existing as an extrachromosomal plasmid. This is a latent form of a phage, in which the viral genes are present in the bacterium without causing disruption of the bacterial cell. Identification of prophage sequences within bacterial genomes and plasmids can be performed using web-based tools which are known by the skilled person of the art. For example, such tools include but are not limited to PHASTER (Arndt et al., 2016 Nucleic Acids Res. 44, W16-W21.), PROPHINDER (Lima-Mendez et al., 2008 Bioinformatics 24, 863-865) or the like. The inventors succeeded to identify sequences on 14 Gardnerella genomes predicted to constitute intact or partial prophages. The sequences were found by identifying DNA regions that cluster genes predicted to be of viral origin. Viral gene clusters predicted to be only partial prophages as opposed to complete prophages were also included.

Then, the putative prophage sequences were annotated by blasting predicted coding sequences, to identify putative endolysins. Specifically, protein sequences homologous to enzymes capable of cleaving any of the key chemical bonds that constitute peptidoglycan were searched. In particular, protein sequences homologous to N-actylmuramidases, N-actylmuramoy-L-alanine amidases, L-alanoyl-D-glutamate endopeptidases, interpeptide bridge endopeptidases, or N-acetyl-beta-D-glucosaminidases were searched. On every individual prophage or partial prophage analyzed, the inventors discovered coding sequences for proteins homologous to 1,4-beta-N-acetylmuramidases and named them EL1 to EL14. The assignment of names to source genomes is shown in Table 1.

TABLE 1

| | Name of the strain |
|---|---|
| Name of the | from which the |
| putative | putative endolysin |
| endolysin | has been identified |
| EL 1 | Strain HMP9231 |
| EL 2 | Strain Gv18-4 |
| EL 3 | Strain Gv18-4 |
| EL 4 | Strain Gv5-1 |
| EL 5 | Strain JCP7276 |
| EL 6 | Strain 1400E |
| EL 7 | Strain AMD |
| EL 8 | Strain JCP7719 |
| EL 9 | Strain 0288E |
| EL 10 | Strain G30-4 |
| EL 11 | Strain JCP8017A |
| EL 12 | Strain 3549624 |
| EL13 | Strain Gv37_1 |
| EL 14 | Strain Gv37_2 |

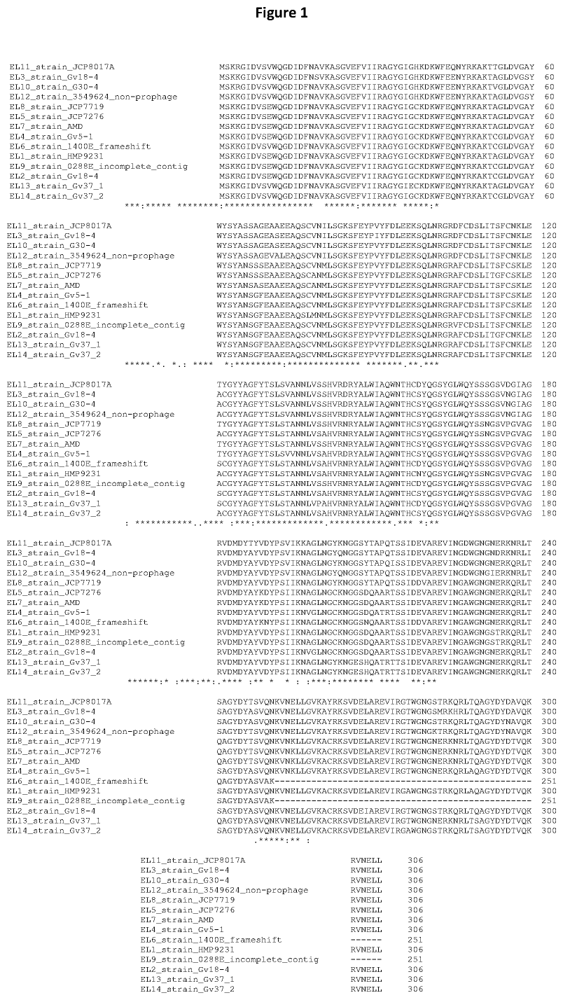

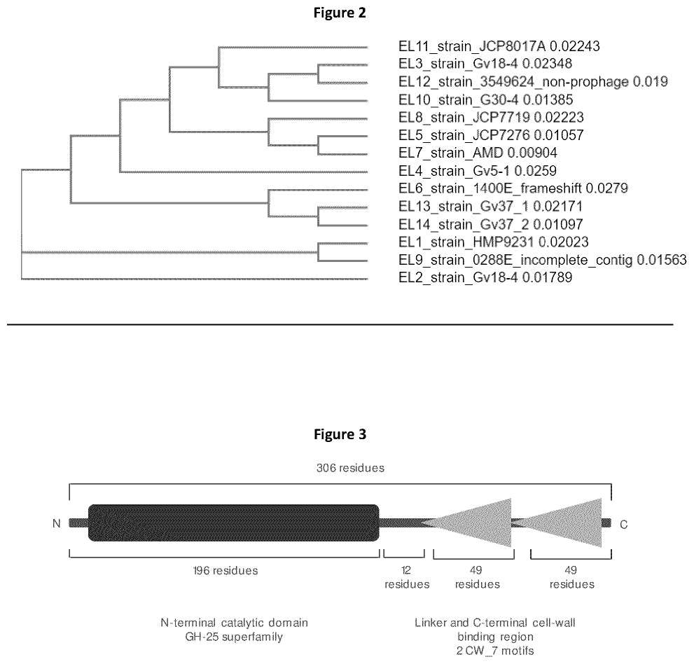

Only one copy per prophage was found in each case, and no other coding sequences predicted to be enzymes capable of lysing the bacterial cell wall were found. The putative 1,4-beta-N-acetylmuramidases were aligned to understand homology and domain structure (see FIG. 1). As can be seen in FIG. 1, the majority of endolysins, even from different prophages on different genomes has exactly 306 residues. The two exceptions are EL6 and EL9. EL6 is truncated at the C-terminus by a frameshift. EL9 ends at the exact same position as EL6, however in this case the whole contig ends. There are no identical pairs among the endolysins, even though they are highly homologous, as can be seen in FIG. 2.

### Example 2—Determination of the Domain Structure of the Natural Gardnerella Prophage Endolysins

The domain structure of the newly discovered endolysins were determined with InterPro (Mitchell et al., 2019 Nucleic Acids Res. 47, D351-D360). Briefly, InterPro is a database of protein families, domains and functional sites in which identifiable features found in known proteins can be applied to new protein sequences in order to functionally characterize them. The contents of InterPro consist of diagnostic signatures and the proteins that they significantly match. The signatures consist of models, e.g. simple types, such as regular expressions or more complex ones, such as Hidden Markov models, which describe protein families, domains or sites. As can be seen in FIG. 3, all endolysins with 306 residues have the same domain arrangement. The N-terminal domain of 196 residues is identified as the catalytic domain, due to its homology to Glycoside hydrolases, family 25. Said catalytic domain is followed by a linker region and two cell-binding domains homologous to the C-terminal domain of lysozyme Cpl-7, also called CW_7 domains (García et al., 1990 Gene 86, 81-88; Lopez and García, 2004 FEMS Microbiol. Rev. 28, 553-580; Bustamante et al., 2010 J. Biol. Chem. 285, 33184-33196, 2012 PLoS One 7, e46654). In the following examples, the above identified catalytic domain represents the “N-terminal catalytic domain” or “H-domain” where, e.g., “H2” refers to the H-domain of the natural EL2. In the following examples, the “linker region” and the “C-terminal cell-wall binding region”, the latter comprising or consisting of one or more cell-wall binding domains or “B-domains”, represent together the “B-region” where, e.g., B10 refers to the B-region of the natural EL10. Likewise, B11_N refers to the N-terminal cell-wall binding domain of natural EL11, B12_C refers to the C-terminal cell-wall binding domain of natural EL12 and so on.

### Example 3—Determination of the Enzymatic Activity of the Natural Gardnerella Prophage Endolysins on Gardnerella Cells

The inventors investigated the enzymatic activity of the newly discovered endolysins against Gardnerella. Whether or not the identified sequences homologous to 1,4-beta-N-acetylmuramidases were active could not be predicted in silico. As it is well-known in the art, bacteria can mutate their prophage sequences and the corresponding prophages might therefore lose their activity to propagate. Moreover, even if the newly discovered phage-encoded peptidoglycan hydrolases were indeed active proteins, it was still not demonstrated that said proteins were able to enzymatically degrade the specific peptidoglycan layer of Gardnerella. Indeed, Gardnerella is special in that it is a Gram-variable species: it does not form the outer membrane defining true Gram-negative species. Its cell wall is generally very thin and has only 10% or less content of peptidoglycan. Thus, a skilled person of the art would have thought that a peptidoglycan-degrading enzyme, such as endolysin proteins, could not efficiently lyse the bacterial cell walls of Gardnerella.

The 14 identified endolysins EL1 to EL14 were cloned with a His-tag, expressed in E. coli and purified via a single-step Ni-NTA column using the method described in Reference Example 1. The assignment of endolysins names to source genome is shown in Table 1. The Gardnerella strains used are shown in Table 2.

TABLE 2

| | Gardnerella strains (new nomenclature following |
|---|---|
| Name | (Vaneechoutte et al., 2019)) |
| Gv_1 | UGent 09.07, strain of G. vaginalis sensu stricto |
| Gv_8 | UGent 25.49, strain of G. vaginalis sensu stricto |
| Gv_9 | ATCC 14018, type strain for G. vaginalis sensu |
| | stricto |
| Gv_10 | UGent 06.41, type strain for G. leopoldii |
| Gv_11 | UGent 09.48, type strain for G. leopoldii |
| Gv_17 | UGent 18.01, type strain for G. piotii |
| Gv_23 | GS 10234 (FC2), type strain for G. swidsinskii |

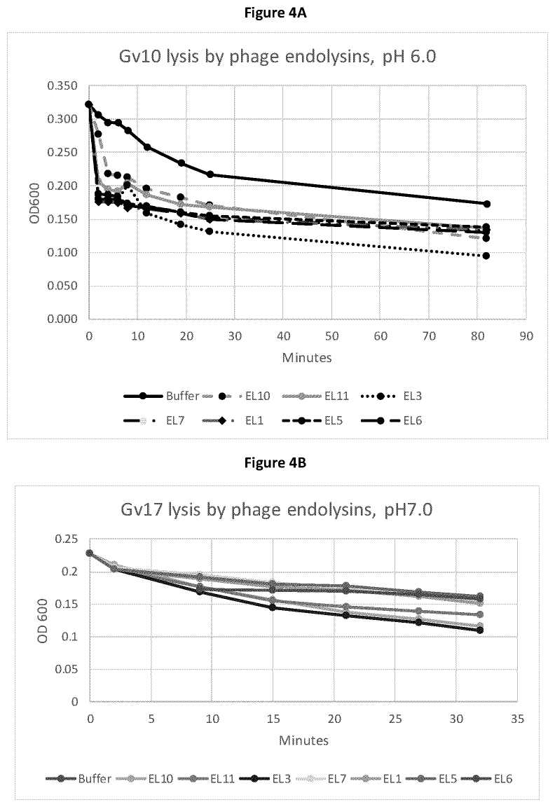

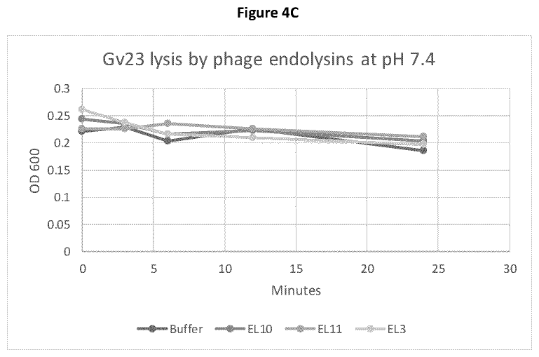

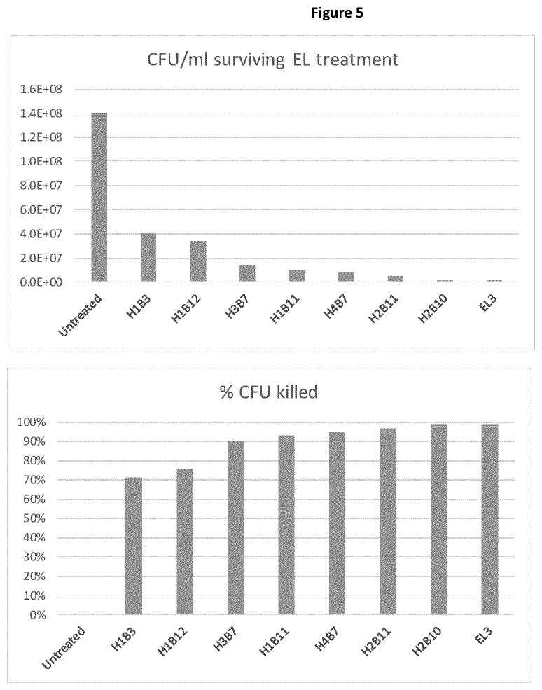

To test the activity of the purified endolysins, the turbidity change of Gardnerella suspensions (see Table 2) was measured at 610-620 nm using essentially the method described in Reference Example 2, where 95 ul of bacterial suspension in Hardy Broth at the indicated pH was mixed with 5 ul of endolysin solution in a photometric cuvette under aerobic conditions at room temperature. In turbidity reduction assays, a decrease in light scattering (i.e., turbidity reduction) of a suspension of live cells can be used in a spectrophotometer to assay the activity of peptidoglycan hydrolases. The reduction in optical density over time (minutes) can be used to calculate a rate of hydrolysis. Results are compared to a “no-enzyme added, buffer only” control preparation treated identically for the same period of time. In this manner, a specific activity of the enzyme preparation can be reported as ΔOD/time/ul lysin protein. As can be seen in FIGS. 4A to 4C, the drop in turbidity was much more pronounced for the endolysin treated groups than for buffer, indicating enzymatic activity. Surprisingly, the inventors therefore discovered that the newly discovered endolysins EL1, EL2, EL3, EL4, EL5, EL7, EL10, EL11 and EL12 were active proteins having the capacity to lyse the Gardnerella cell walls. As explained above, due to the low content of peptidoglycan in the cell wall, the fact that a peptidoglycan-degrading enzyme such as the identified endolysins could efficiently lyse the bacterial cell walls was an unexpected and surprising discovery.

### Example 4—Identification of the Most Active Domains with Artificial Domains-Swapped Endolysins

Whether the different N-terminal enzymatic domains could have different lytic activities and whether the B-region (comprising the linker and the cell-wall binding domains) could mediate specificity to different strains was then assessed by the inventors. For that purpose, domain-swapped endolysins were artificially generated.

4.1—Endolysins Constructs

To artificially generate the domains-swapped endolysins, the N-terminal 196 residues of a first natural endolysin, comprising the catalytic domain, were swapped as a block against the full C-terminal region of 110 residues of a second natural endolysin, comprising the linker region and two cell-wall binding domains. Domain-swapped proteins were prepared by performing the following methods. The original constructs EL1-14 were ordered from GeneWiz as synthetic genes with codon-optimization for E. coli. These constructs were cloned by GeneWiz into the pETM14_ccdB vector via restriction/ligation approach using the recognition sites for NcoI and NotI enzymes.

Table 3 below summarizes the primers used. For domain swapping of the selected 10 constructs, each H-domain together with the T7 promoter was amplified by a common forward primer (no 2) and a construct-specific reverse primer (no 3-12) using the PhusionFlash polymerase (Thermo, F-548L). Similarly, each B-region was amplified by a construct-specific internal primer (no 13-21) and a common reverse primer (no 1) including the T7 terminator. All primers contained extensions bearing the BsaI recognition site, making the outer ends compatible with the pETM14-derived vector backbone pETMdest. The overhang between the domains was designed to be of sequence “GGCT” within the two amino acids GL of the linker sequence. These 2 amino acids therefore represented the exact border between the domains for the purpose of this experiment.
Thus amplified and gel-purified domains (GeneJet Gel purification kit, Thermo, K0692) were then combined into 90 new expression constructs using the GoldenGate cloning strategy by BsaI restriction (BsaI-HFv2, NEB, R3733S)/ligation (T4 DNA Ligase, Thermo, EL0011) cycling reaction.
For transformation purposes, the NEB10beta E. coli strain was used (NEB, C3019) and plasmids were purified using the GeneGet Plasmid Miniprep Kit (Thermo, K0502).

TABLE 3

| | | |
|---|---|---|
| 1 | T7casette-16-rev | aacaggtctcaatacaatccggatatagttcctcctttcagc |
| 2 | T7casette-01-for | aacaggtctcaacctccgcgaaattaatacgactcactatagg |
| 3 | EL_H1-rev | aacaggtctcaagccggcatttttgatgatgctcggg |
| 4 | EL_H2-rev | aacaggtctcaagccaacatttttaataatgctcggataatcc |
| 5 | EL_H3-rev | aacaggtctcaagccggcgtttttgataacgctcggg |
| 6 | EL_H4-rev | aacaggtctcaagcccacgttcttgatgatgctcgg |
| 7 | EL_H5-rev | aacaggtctcaagccggcgtttttgatgatgctcgg |
| 8 | EL_H6-rev | aacaggtctcaagccggcattcttaataatgctcggata |
| 9 | EL_H7-rev | aacaggtctcaagccggcgtttttaatgatgctcggataatc |
| 10 | EL_H10-rev | aacaggtctcaagccggcgtttttgatcacgctcgg |
| 11 | EL_H11-rev | aacaggtctcaagccggccttcttgataacgctcggataatc |
| 12 | EL_H12-rev | aacaggtctcaagccggcattcttgatcacgctcgg |
| 13 | EL_B6-for | aacaggtctcaggcttaaacggctgcaaaaatggcgg |
| 14 | EL_B7-for | aacaggtctcaggcttaaacggctgcaaaaacggtgg |
| 15 | EL_B10-for | aacaggtctcaggcttaaacggctataaaaacggcggc |
| 16 | EL_B11-for | aacaggtctcaggcttaaatggttacaagaatggcggcag |
| 17 | EL_B12-for | aacaggtctcaggcttaaatggctaccagaacggcgg |
| 18 | EL_Bl-for | aacaggtctcaggcttaaacggctgcaagaatggtgg |
| 19 | EL_B2-for | aacaggtctcaggcttaaatggttgcaagaacggcgg |
| 20 | EL_B3-for | aacaggtctcaggcttaaatggctaccagaatggcggc |
| 21 | EL_B4-for | aacaggtctcaggcttaaatggctgcaaaaacggtggc |
| 23 | T7term-STOP-for | aacaggtctcatgacgccattaacctgatgttctggg |

For ease of reference, the domain combination of the artificial endolysins is expressed with a H-code for the N-terminal catalytic domain (thereafter called the H-domain) and a B-code for the part comprising the C-terminal cell-wall binding region and the linker region (thereafter called the B-region). By way of example, H2B10 refers to a domain-swapped endolysin with the N-terminal domain from the natural endolysin EL2, and the linker region and C-terminal cell-wall binding region from the natural endolysin EL10. In other words, H2B10 refers to a domain-swapped endolysin consisting of the 196 N-terminal residues of the natural endolysin EL2 (SEQ ID NO: 2) and the 110 C-terminal residues of the natural endolysin EL10. In this example, the B-region B10 corresponding to the 110 C-terminal residues of the natural endolysin EL10, comprises from the C-terminal to the N-terminal order, a C-terminal cell-wall binding domain “B10_C” (SEQ ID NO: 29), a N-terminal cell-wall binding domain “B10_N” (SEQ ID NO: 28) and a linker region “L10” (NAGLNGYKNGGS). The nomenclature and the corresponding amino acid sequences are displayed in details in Table 7.
Therefore, according to above nomenclature, a natural endolysin, e.g. EL3, can be defined either as H3B3 or H3-L3-(B3_N)(B3_C) interchangeably. Likewise, a recombinant endolysin, e.g. H2B10, can also be defined as H2-L10-(B10_N)(B10_C) interchangeably.
A skilled person of the art would understand that the elements “Lx”, “(Bx_N)” and “(Bx_C)” might also be swapped independently in other embodiments. The nomenclature is further displayed in Table 7, below.

4.2—Optimization of Assay Parameters

The dependence of activity on three potentially critical parameters was analyzed, i.e. pH, anerobic/micro-aerophilic/aerobic conditions, absence/presence of imidiazole. The three criteria to assess have been selected for the following reasons.

pH: The killing activity of the endolysins of the invention has been successfully demonstrated with experiments conducted at pH values around 7. However, the pH in a healthy vagina is about 3.5 while in a BV vagina the pH is up to about 5.5 and even higher. Therefore, the pH-dependence of the endolysins activity has been investigated.

Oxygen: In literature, Gardnerella is described as anaerobic or micro-aerophilic. Therefore, it has been investigated whether more untreated cells survived under anaerobic, micro-aerophilic or aerobic conditions for the incubation period of the experiment (usually 5 hours).

Imidazole: According to the method described in Reference Example 1, the endolysins of the invention are purified via a one-step Ni-NTA column, where the buffer used to elute the endolysins from the Ni-NTA matrix contained Imidazole. Therefore, in the absence of a further step of dialyzing the sample, the obtained eluate solutions contain 250 mM Imidazole. In that respect, the effect of imidazole on Gardnerella has been investigated.

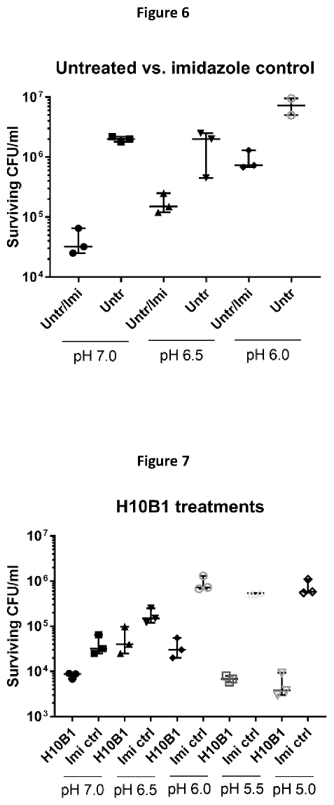

First, the sensitivity of G. vaginalis Gv_9 survival to incubation in medium with or without imidazole at different pH values was assessed (see FIG. 6). 5×10⁷ CFU/ml cells were incubated under the conditions indicated below the graph for 5 hours at 37° C. under anaerobic conditions. Then, the surviving CFU/ml was determined by quantitative plating. As depicted in FIG. 6, at pH 6.0, a median of 1×10⁷ and 1×10⁶ cells survive the incubation without and with imidazole, respectively. At pH 7.0, only medians of 2×10⁶ and 3×10⁴ cells survive without and with imidazole, respectively. In the untreated control at pH 5.0, 1e7 cells survive the procedure, while the median survival of Imidazole treated at pH 7 is 3e4, i.e. 3 logs below the former. Therefore, the survival of G. vaginalis Gv_9 is highly dependent on the absence of imidazole, especially at pH>6.0, and of a low pH.
Second, the sensitivity of G. vaginalis Gv_9 to treatment with the recombinant endolysin H10B1 against control containing imidazole at different pH values was assessed (see FIG. 7). 5×10⁷ CFU/ml cells were incubated under the conditions indicated below the graph for 5 hours at 37° C. under anaerobic conditions. Then, the surviving CFU/ml was determined by quantitative plating. The columns labeled imidazole control depict the same data as in FIG. 6. As depicted in FIG. 7, the endolysin is highly active down to pH 5.0, and even the relative reduction vs. control is much more pronounced at this low pH, with a reduction in viable CFU of 2.5 logs. While at pH 7.0 there was less than 1 log 10 difference in survival between H10B1-treated and untreated cells, the difference was 2 log 10 units at pH 5.0. The survival of cells not treated with H10B1 did not increase at pH values lower than 6.0 in presence of imidazole. Therefore, the activity of the endolysin H10B1 is highly pH dependent. When similar experiments were conducted under aerobic conditions, the survival of control cells was reduced by several log 10 units compared to anaerobic conditions (data not shown).
Therefore, it has been concluded that the optimized parameters to conduct the experiments with the endolysins of the invention were under anaerobic conditions, at pH 5.0, and with a step of removing imidazole from the endolysin eluates.

4.3—Expression Levels

Table 4 depicts an overview of the concentrations of all endolysin constructs. Each construct that had a concentration above 0.2 mg/ml after the removal of imidazole were adjusted to a concentration of 0.2 mg/ml by dilution. Constructs with a lower concentration were left as is and tested for their activity. Natural endolysin EL6 (not shown in Table 4) had a concentration below 0.2 mg/ml. H4, H11 and H12 appear to confer low solubility and expression levels, as most constructs fell under the threshold of 0.2 mg/ml. Also for H1 several constructs had a low concentration.

TABLE 4
Overview of expression levels (quantitative)

| | B1 | B2 | B3 | B4 | B5 | B7 | B10 | B11 | B12 |
|---|---|---|---|---|---|---|---|---|---|
| H1 | 0.2 | | | | 0.2 | 0.2 | 0.2 | 0.2 | |
| H2 | 0.2 | 0.2 | 0.2 | 0.2 | 0.2 | 0.2 | 0.2 | 0.2 | 0.2 |
| H3 | 0.2 | 0.2 | 0.2 | 0.2 | 0.2 | 0.2 | 0.2 | 0.2 | 0.2 |
| H4 | | | | | | | 0.2 | 0.2 | |
| H5 | 0.2 | 0.2 | 0.2 | 0.2 | | 0.2 | 0.2 | 0.2 | 0.2 |
| H7 | | 0.2 | 0.2 | 0.2 | 0.2 | 0.2 | 0.2 | 0.2 | 0.2 |
| H10 | 0.2 | 0.2 | 0.2 | 0.2 | 0.2 | 0.2 | 0.2 | 0.2 | 0.2 |
| H11 | | | 0.2 | | | | 0.2 | | 0.2 |
| H12 | | | | | | | | | |

4.4—Quantitative Assessment of the Lysis of the Four Main Species of Gardnerella by Endolysin Action in Suspension Under Optimized Conditions

The lysis activity on the four main species of Gardnerella of 91 constructs (natural and domain-swapped endolysins) was quantitatively assessed using the method described in Reference Example 2. Briefly, 90 ul 5e7 CFU/ml of the indicated strain were incubated for 5 hours at pH 5.0 under anaerobic conditions with 10 ul endolysin (concentration adjusted to 0.2 mg/ml where possible, see Table 4).

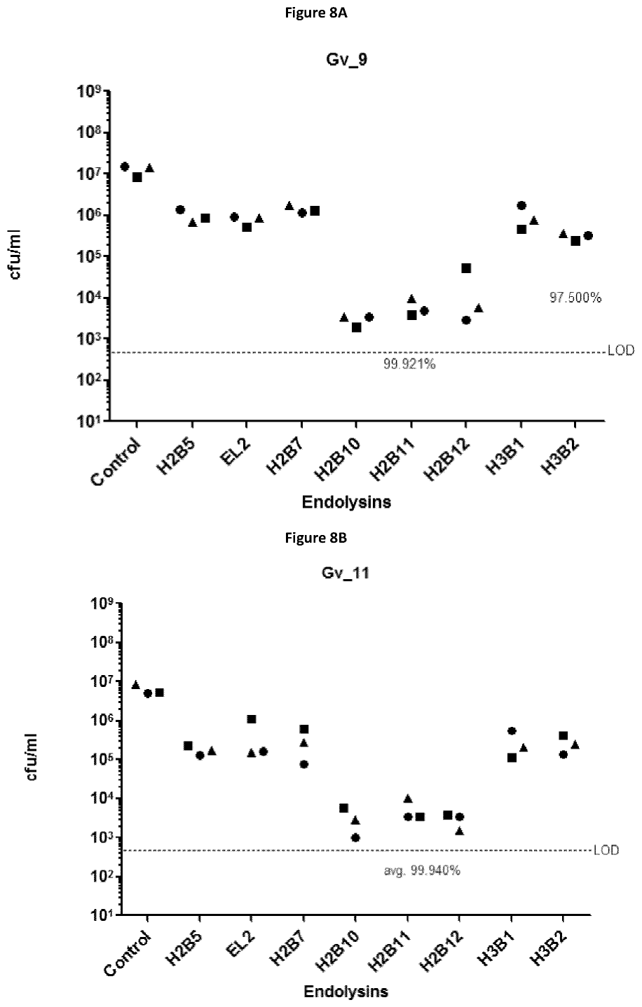

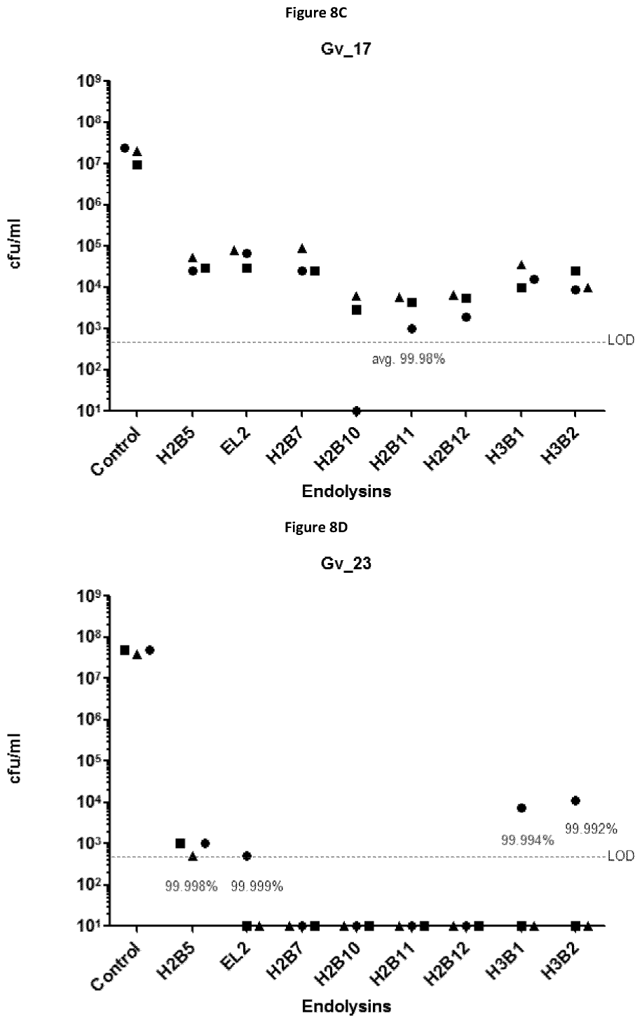

The results are shown in FIGS. 8A to 8D together with Tables 5A to 5C. In FIGS. 8A to 8D, the logarithmic Y axis depicts the count of surviving cells. The dotted line indicates the limit of detection (LOD) given by plating of 2 ul of the reaction mix (500 CFU/ml). Each combination of the natural 10 H-domains and the natural 9 B-regions was assessed, including the natural endolysins (H1B1, H2B2, H3B3, etc.), plus H6B6. No other constructs with the B-domain B6 were tested, as B6 made all H-domains fused to it inactive, as determined by OD measurement (data not shown). The activity of each construct was measured on each of the 4 main Gardnerella species G. vaginalis sensu stricto, G. leopoldii, G. piotii, and G. swidsinkii. Table 5 summarizes the resulting log 10 reduction of CFU, organized by H-domain and B-region. All conditions have been measured in triplicate. The survival after 5 hours incubation at pH 5.0 under anaerobic conditions with endolysins vs. buffer was measured by quantitative plating. The values indicate the log 10 of the ratio of surviving CFU for treated vs. untreated cells. The average of the triplicate measurements is used. In tables 5A and 5B, high negative log 10 values, e.g. −6.7, −5.5, −4.8 etc. are associated with high enzymatic activity, while log 10 values closer to zero or even positive log 10 values are associated with low or no enzymatic activity. To provide an example, if the average CFU of the 3 control treated measurements on Gv_9 were 1.0×10⁷ CFU/ml, and the average of the 3 samples treated with H2B10 was 2.5×10³ CFU/ml, then the log 10 value of the CFU reduction of Gv_9 by H2B10 would be log 10(2×10³/10⁷)=−3.7. Inversely, a reduction value of −3.7 means a 10^{3.7}-fold (=5012-fold) reduction of viable CFU in treated sample vs. untreated control. In case of an inactive endolysin, e.g. H4B3 on Gv_9, the Gv_9 CFU after treatment would be equal to the CFU measured in the control treated sample and the ratio of the two CFU values would be one. Therefore, the reduction value of H4B3 on Gv_9 is log 10(1)=0.0.

TABLE 5A

| | B1 | | | | B2 | | | | B3 | | | | B4 | | | | B5 | | | |
|---|---|---|---|---|---|---|---|---|---|---|---|---|---|---|---|---|---|---|---|---|
| | Gv9 | Gv 11 | Gv17 | Gv23 | Gv9 | Gv 11 | Gv17 | Gv23 | Gv9 | Gv 11 | Gv17 | Gv23 | Gv9 | Gv 11 | Gv17 | Gv23 | Gv9 | Gv 11 | Gv17 | Gv23 |
| H1 | −1.9 | −1.3 | −0.9 | −3.5 | −0.4 | −0.3 | −0.2 | −1.4 | −0.7 | −0.7 | −0.3 | −1.9 | −0.8 | −0.7 | −0.7 | −2.3 | −1.8 | −0.8 | −0.8 | −3.3 |
| H2 | −1.6 | −1.2 | −1.0 | −3.5 | −1.2 | −1.1 | −2.5 | −5.4 | −2.9 | −2.1 | −1.6 | −5.4 | −2.3 | −1.8 | −1.6 | −4.5 | −1.1 | −1.5 | −2.7 | −4.7 |
| H3 | −1.1 | −1.3 | −2.9 | −4.3 | −1.6 | −1.4 | −3.1 | −5.4 | −3.0 | −2.0 | −1.5 | −5.4 | −1.9 | −1.4 | −1.2 | −4.0 | −1.4 | −1.2 | −0.6 | −3.5 |
| H4 | 0.1 | 0.7 | 0.4 | 0.0 | 0.1 | 0.5 | 0.4 | 0.0 | 0.0 | 0.4 | 0.2 | −0.3 | −0.4 | −0.5 | −0.4 | −2.0 | 0.1 | 0.3 | 0.5 | 0.1 |
| H5 | −1.3 | −1.5 | −1.1 | −3.8 | −1.2 | −1.3 | −0.9 | −3.3 | −2.8 | −3.0 | −1.8 | −4.8 | −1.8 | −3.0 | −2.2 | −4.5 | 0.2 | 0.3 | −0.1 | −0.1 |
| H6 | −0.3 | −0.1 | −0.4 | −1.8 | −0.8 | −0.4 | −0.7 | −2.4 | −0.3 | −0.5 | −0.3 | −1.6 | −0.4 | −0.7 | −0.3 | −1.6 | −0.4 | −0.5 | −0.4 | −1.7 |
| H7 | −0.9 | −1.4 | −0.9 | −3.1 | −2.0 | −2.2 | −1.3 | −4.2 | −3.2 | −3.3 | −2.0 | −4.8 | −2.5 | −3.1 | −1.7 | −4.0 | −1.6 | −2.1 | −1.2 | −3.2 |
| H10 | −1.7 | −1.1 | −1.4 | −3.2 | −1.6 | −1.2 | −1.2 | −3.9 | −2.7 | −1.9 | −2.1 | −4.9 | −2.3 | −1.5 | −1.7 | −4.2 | −1.2 | −0.8 | −1.2 | −3.5 |
| H11 | −0.8 | −1.3 | −0.8 | −2.3 | −0.2 | −0.4 | −0.4 | −0.3 | −2.5 | −2.2 | −1.7 | −4.2 | −1.7 | −1.7 | −1.1 | −2.8 | −1.5 | −1.7 | −0.9 | −3.2 |
| H12 | 0.0 | −0.8 | −0.2 | −1.5 | −0.2 | −0.9 | −0.3 | −2.1 | −0.2 | −1.2 | −0.6 | −2.5 | 0.0 | −0.7 | −0.3 | −2.3 | −0.2 | −1.1 | −0.4 | −2.1 |
| | | | | | | | | | B6 | | | | B7 | | | | B10 | | | |
| | | | | | | | | | Gv9 | Gv 11 | Gv17 | Gv23 | Gv9 | Gv 11 | Gv17 | Gv23 | Gv9 | Gv 11 | Gv17 | Gv23 |
| | | | | | | | | H1 | | | | | −1.6 | −1.2 | −0.9 | −3.3 | −2.5 | −2.3 | −1.5 | −4.9 |
| | | | | | | | | H2 | | | | | −1.0 | −1.3 | −2.6 | −6.7 | −3.6 | −3.3 | −3.8 | −6.7 |
| | | | | | | | | H3 | | | | | −0.9 | −1.1 | −0.3 | −2.8 | −1.5 | −2.2 | −0.6 | −3.8 |
| | | | | | | | | H4 | | | | | 0.1 | 0.1 | −0.4 | −1.3 | −3.0 | −2.9 | −1.9 | −4.8 |
| | | | | | | | | H5 | | | | | −1.6 | −2.2 | −1.2 | −4.2 | −2.5 | −3.6 | −1.1 | −4.7 |
| | | | | | | | | H6 | 0.1 | 0.2 | −0.2 | −0.1 | −0.4 | −0.8 | −0.6 | −2.0 | −0.5 | −0.7 | −0.6 | −1.8 |
| | | | | | | | | H7 | | | | | −1.6 | −2.1 | −1.2 | −3.2 | −3.4 | −3.0 | −1.8 | −5.1 |
| | | | | | | | | H10 | | | | | −1.4 | −0.8 | −1.2 | −3.5 | −3.5 | −1.6 | −2.4 | −5.5 |
| | | | | | | | | H11 | | | | | −0.1 | −0.9 | −0.7 | −1.9 | −2.8 | −2.2 | −1.4 | −4.1 |
| | | | | | | | | H12 | | | | | −0.8 | −0.6 | −0.4 | −2.6 | −1.3 | −0.3 | −0.4 | −2.9 |
| | | | | | | | | | | | | | B11 | | | | B12 | | | |
| | | | | | | | | | | | | | Gv9 | Gv 11 | Gv17 | Gv23 | Gv9 | Gv 11 | Gv17 | Gv23 |
| | | | | | | | | | | | | H1 | −1.5 | −1.6 | −0.8 | −3.8 | −0.7 | −0.6 | −0.4 | −2.3 |
| | | | | | | | | | | | | H2 | −3.3 | −3.0 | −3.7 | −6.7 | −2.8 | −3.3 | −3.6 | −6.7 |
| | | | | | | | | | | | | H3 | −1.6 | −2.0 | −0.8 | −4.2 | −1.3 | −1.9 | −0.5 | −3.7 |
| | | | | | | | | | | | | H4 | −2.9 | −3.3 | −1.8 | −4.8 | −0.3 | −1.1 | −0.3 | −3.9 |
| | | | | | | | | | | | | H5 | −2.4 | −1.6 | −1.6 | −4.4 | −2.3 | −1.9 | −1.5 | −4.3 |
| | | | | | | | | | | | | H6 | −1.0 | −1.5 | −0.7 | −2.7 | −0.4 | −0.6 | −0.3 | −1.3 |
| | | | | | | | | | | | | H7 | −3.5 | −3.3 | −1.9 | −3.7 | −2.6 | −3.3 | −1.7 | −4.1 |
| | | | | | | | | | | | | H10 | −3.2 | −1.7 | −1.8 | −4.6 | −3.6 | −2.8 | −1.6 | −5.0 |
| | | | | | | | | | | | | H11 | 0.1 | −1.6 | −1.0 | −3.5 | −3.0 | −2.3 | −1.3 | −4.1 |
| | | | | | | | | | | | | H12 | −2.4 | −1.0 | −1.0 | −3.9 | −0.2 | −1.0 | −0.3 | −1.9 |

Table 5B below depicts the same data as Table 5A, but displayed as averages of the log 10 activities of each construct across the four Gardnerella strains. At the right and bottom, average values of each natural H-domain across all natural B-regions (except B6), and each natural B-region (except B6) across all natural H-domains, respectively, are shown along with the activity rank of the respective natural H-domain and natural B-region.

TABLE 5B

| | B1 | B2 | B3 | B4 | B5 | B6 | B7 | B10 | B11 | B12 | Avg | Rank |
|---|---|---|---|---|---|---|---|---|---|---|---|---|
| H1 | −1.9 | −0.6 | −0.9 | −1.1 | −1.7 | | −1.8 | −2.8 | −1.9 | −1.0 | −1.5 | 7 |
| H2 | −1.8 | −2.6 | −3.0 | −2.5 | −2.5 | | −2.9 | −4.3 | −4.2 | −4.1 | −3.1 | 1 |
| H3 | −2.4 | −2.5 | −3.0 | −2.2 | −1.7 | | −1.3 | −2.0 | −2.1 | −1.9 | −2.1 | 5 |
| H4 | 0.3 | 0.3 | 0.1 | −0.8 | 0.3 | | −0.4 | −3.1 | −3.2 | −1.4 | −0.9 | 9 |
| H5 | −1.9 | −1.7 | −3.1 | −2.9 | 0.1 | | −2.3 | −3.0 | −2.5 | −2.5 | −2.2 | 4 |
| H6 | −0.7 | −1.1 | −0.7 | −0.8 | −0.8 | 0.0 | −1.0 | −0.9 | −1.5 | −0.7 | −0.8 | 10 |
| H7 | −1.6 | −2.4 | −3.4 | −2.8 | −2.0 | | −2.0 | −3.3 | −3.1 | −3.0 | −2.6 | 2 |
| H10 | −1.9 | −2.0 | −2.9 | −2.4 | −1.7 | | −1.7 | −3.3 | −2.9 | −3.3 | −2.4 | 3 |
| H11 | −1.3 | −0.3 | −2.6 | −1.8 | −1.8 | | −0.9 | −2.6 | −1.5 | −2.7 | −1.7 | 6 |
| H12 | −0.6 | −0.9 | −1.1 | −0.8 | −1.0 | | −1.1 | −1.2 | −2.1 | −0.9 | −1.1 | 8 |
| Avg | −1.4 | −1.4 | −2.1 | −1.8 | −1.3 | | −1.5 | −2.7 | −2.5 | −2.1 | | |
| Rank | 7 | 8 | 4 | 5 | 9 | | 6 | 1 | 2 | 3 | | |

Table 5C depicts the activity ranks of all endolysins, based on the data of Table 5B.

TABLE 5C

| | B1 | B2 | B3 | B4 | B5 | B6 | B7 | B10 | B11 | B12 |
|---|---|---|---|---|---|---|---|---|---|---|
| H1 | 44 | 83 | 72 | 63 | 53 | | 50 | 21 | 43 | 67 |
| H2 | 49 | 25 | 12 | 26 | 29 | | 17 | 1 | 2 | 3 |
| H3 | 33 | 27 | 13 | 35 | 52 | | 61 | 39 | 36 | 46 |
| H4 | 91 | 90 | 88 | 76 | 89 | | 84 | 9 | 8 | 59 |
| H5 | 42 | 55 | 11 | 18 | 87 | | 34 | 14 | 30 | 28 |
| H6 | 80 | 66 | 79 | 78 | 77 | 86 | 68 | 71 | 58 | 81 |
| H7 | 56 | 32 | 4 | 20 | 38 | | 40 | 5 | 10 | 15 |
| H10 | 45 | 41 | 16 | 31 | 54 | | 51 | 6 | 19 | 7 |
| H11 | 60 | 85 | 24 | 47 | 48 | | 70 | 23 | 57 | 22 |
| H12 | 82 | 74 | 64 | 75 | 69 | | 65 | 62 | 37 | 73 |

Table 5D depicts the average log 10 lysis of each Gardnerella strain used. The averages were calculated of the log 10 activities across all constructs tested.

TABLE 5D
Log10 average activity across all constructs by strain

| | |
|---|---|
| Gv_9 (G. vaginalis) | −1.5 |
| Gv_11 (G. leopoldii) | −1.5 |
| Gv_17 (G. piotii) | −1.1 |
| Gv_23 (G. swidsinkii) | −3.4 |

Tables 5A to 5C show that the activity of the endolysins was highly specific dependent on the H-domain/B-region combination and the bacterial strain it was tested on. Each construct was assayed against the four Gardnerella strains (see Table 5A). On average, the most active H-domain is H2, with an average reduction of 3.1 log 10 units of CFU across all B-regions (except B6), followed by H7, H10 and H5 (see Table 5B). Of the B-regions, B10 is the most active, with an average CFU reduction of 2.7 log₁₀ units, followed by B11, B12, and B3.
Surprisingly and unexpectedly, the inventor discovered that several recombinant endolysins have a stronger activity than any natural endolysin (H1B1 to H12B12), especially when viewed across all 4 Gardnerella strains tested (see FIGS. 8A to 8D). Particularly H2B10, H2B11, and H2B12 have activity ranks 1, 2, and 3, respectively, and each is more active than any natural endolysin (see Table 5C). H7B3 has rank 4 overall (see Table 5C) and is also more active than any other natural endolysin included in the experiment. In fact, the only natural endolysin ranking within the 10 most active is H10B10 (rank 6), the next most active natural endolysin being H3B3 (rank 13). In summary, recombinant endolysins according to the present disclosure might exhibit significantly higher activity than the natural endolysins.
While not being restricted to a particular theory, the unexpected increase of killing activity against Gardnerella observed by domain-swapping the endolysins according to the invention can be explained as follows. Natural endolysins on prophages undergo a Darwinian evolution process, where the propagation of the whole prophage is being optimized. However, the probability of mutations that lead to higher propagation of the respective prophage by simultaneously improving the catalytic activity of the endolysin and at the same time broadening its host range across species within only Gardnerella is very low. In the contrary, some Gardnerella prophages must have evolved the N-terminal catalytic domain of the endolysin to highest activity, and some other prophages must have optimized the C-terminal region of the endolysin for broadest activity across Gardnerella species. Therefore, by combining an highly evolved N-terminal catalytic domain of one of the endolysins of the invention with an highly evolved C-terminal region of another endolysin of the invention encoded by a different genome from a different prophage, recombinant endolysins with higher optimized killing activity against Gardnerella species than the natural endolysins of the invention can be achieved. This is for example achieved by the recombinant endolysins of the invention H2B10, H2B11, H2B12, and H7B3, which all have a higher killing activity than the natural endolysins EL2 or any other natural endolysins, and that across all Gardnerella species (see FIGS. 8A to 8D and Tables 5A).
Moreover, most constructs are much more active on Gv_23 (G. swidsinskii) than on the other three strains tested (see Table 5A). The average activity across all constructs on each Gardnerella strain (see Table 5D) confirms that Gv_23 is the most susceptible to endolysins, followed by Gv_9 and Gv_11, while Gv_17 (G. piotii) is the least susceptible. This order of susceptibility is mostly the case across endolysin constructs. While Gv_23 is the most susceptible strain, for many constructs by several log 10 units, sometimes Gv_17 is more susceptible than Gv_9 or Gv_11 (e.g. for H2B10, the most active endolysin overall). Without being bound by a particular theory, the susceptibility difference can be explained by either a structural deficits like a weaker/thinner/more accessible cell wall of Gv_23, or a stronger enzymatic activity on Gv_23 of the endolysins tested.
Furthermore, it can be concluded that the concentration of the endolysins in solution is critical for their activity in the assay. The constructs with low concentration as depicted in Table 4 generally also have a low activity in the activity assay—particularly the ones with the H-domains H4, H11 and H12 (which confer low solubility across B-regions). There are few surprises, like H12B11, which had a very low expression level but comparably high activity.

4.5—Activity Pattern Analysis

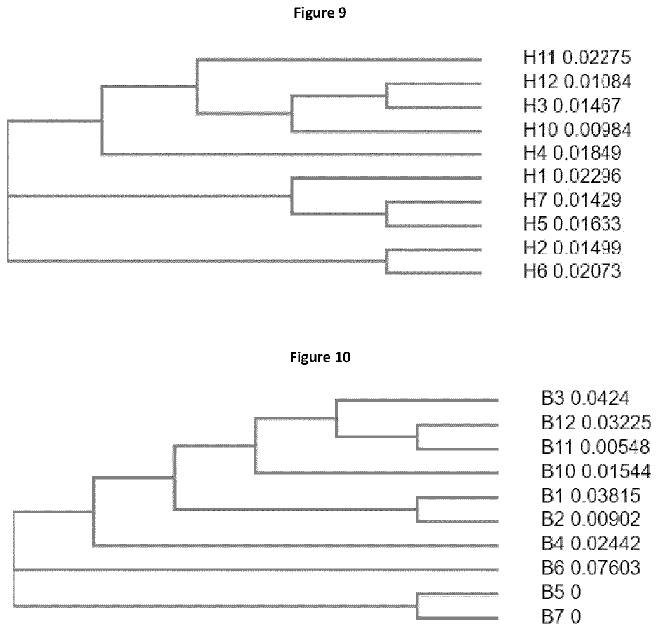

The sequences of the natural H-domains were aligned and compared to relate to the activity patterns, as depicted in the dendrogram of FIG. 9. It was expected that the most active H-domains are also most closely related to each other. However, surprisingly, the most active N-terminal domain, H2, is most homolog to H6, which is the least active. The next-most active H-domains, H7 and H10, are also rather distantly related to each other and to H2. The fourth-most active H-domain, H5, is most closely related to H7. Also the combinations with the B-regions which make the recombinant endolysins most active do not lead to a predictable pattern. H2 is most active in combination with B10, B11 and B12. However the second most active H-domain, H7, is most active in combination with B3, as is the case for its closest homolog, H5.

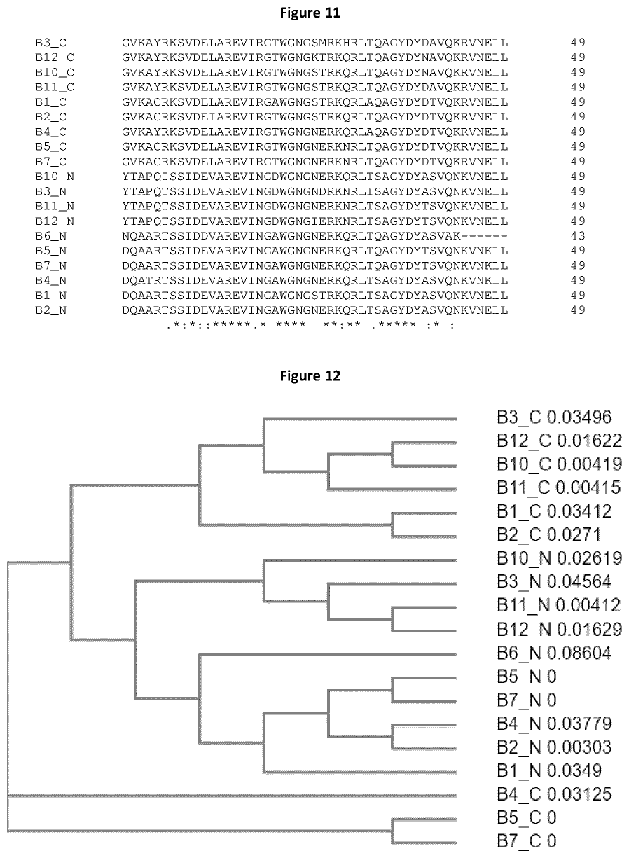

Also the B-regions were aligned to reconcile homologies to the activity pattern, as depicted in the dendrogram of FIG. 10. The most active B-regions as of the analysis in Tables 5A to 5C are B10, B11, B12, followed by B3, which all have average CFU reduction values above 2 log 10 units. In contrast to the pattern seen for H-domains, these 4 most active B-regions are the 4 closest homologs within the group of tested B-regions. Interestingly, the B5 and B7 regions are identical (see FIG. 10). The best overall results were obtained for H2B10, H2B11 and H2B12 as can be seen in FIGS. 8A to 8D.
As explained in Example 2, each natural B-region comprises two B-domains, namely a N-terminal cell-wall domain and a C-terminal cell-wall domain. The sequence of each natural B-domain within the B-region were also aligned and compared, as depicted in FIGS. 11 and 12. The boundaries of the B-domains can be identified both by analyzing the sequence with Interpro (Mitchell et al., 2019 Nucleic Acids Res. 47, D351-D360)) and by aligning the two repetitive motifs within each B-region. The C-terminus of all B-domains is a conserved sequence (VNELL or VNKLL), homologous to which can be found also at the C-terminus of the CW_7 motifs (VNELL or VNEIL) of the protein Cpl-7, thereby defining the boundaries of the two B-domains in each B-region. As an exception, B6 has only one truncated B-domain, which is likely to be the reason for the complete inactivity of EL6.
Concluding, the specific combination of H-domain and B-region has been shown to be critical and each of the H/B combinations leading to endolysins with higher killing activities compared to the natural endolysins was a surprising and non-predictable discovery.

### Example 5—Activity Assay Against Beneficial Lactobacilli

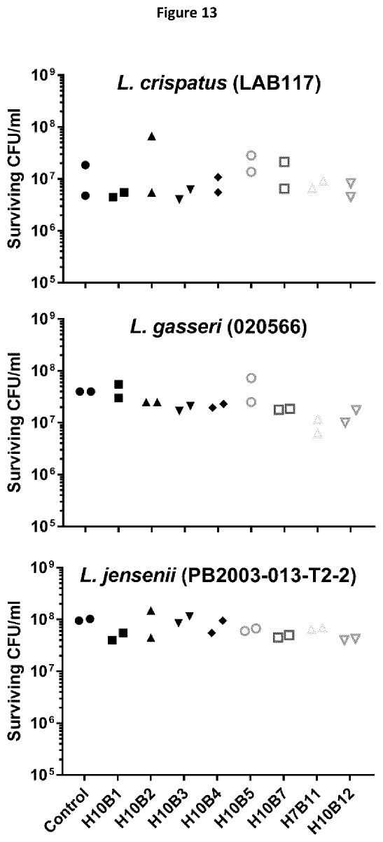

The healthy vagina is populated mainly by 3 species of Lactobacilli: L. crispatus, L. gasseri and L. jensenii. These maintain an acidic pH of 3.5-4.5, by producing lactic acid, and a protective oxidative milieu, by producing H₂O₂. Recovery from BV is associated with a re-population of the vagina with these Lactobacilli, and a pharmaceutical against BV should advantageously not interfere with this process. Antibiotics obviously do, which is why there is still a strong medical need for improved methods and compositions to treat Gardnerella infections and BV. After having successfully demonstrated the high activity of the endolysins of the invention on Gardnerella, the inventors investigated whether those endolysins can lyse strains of the 3 most frequent Lactobacilli species in the healthy vagina. The experiment has been performed using the method described in Reference Example 2 at pH 5.0, under anaerobic conditions. As depicted in FIG. 13, the recombinant endolysins tested do not exhibit any killing activity against the three species of beneficial Lactobacilli used, namely L. crispatus, L. gasseri and L. jensenii. The endolysins of the invention, although exhibiting a high killing activity against Gardnerella, are ineffective against the most frequent beneficial Lactobacilli. These results therefore confirm the genus-selective activity of the endolysins of the invention and their drug candidate status as an innovative pharmaceutical against BV. In that respect, treating BV with the endolysins of the invention is far advantageous to the currently available treatments, such as the treatments with the antibiotics Metronidazole and Clindamycin.

### Example 6—Activity Assays of Standard of Care Antibiotics Metronidazole and Clindamycin on the Growth in Suspension of the Gardnerella Strains

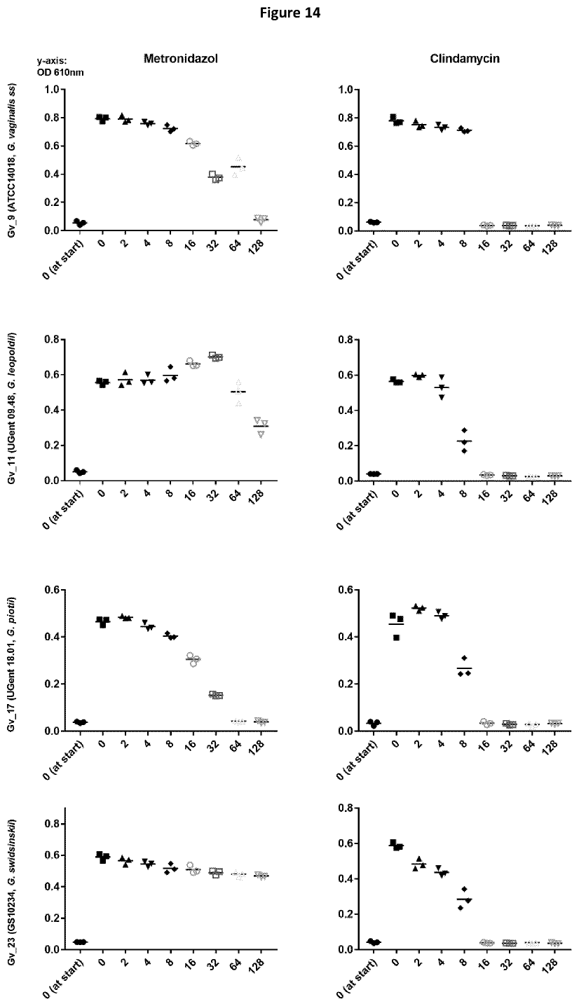

One of the main deficiencies in the treatment of BV is the high rate of recurrence in many women, which leads to repeated administration of antibiotics and concomitant destabilization of the microbiome and other side effects. The minimal inhibitory concentration (MIC) and minimal bactericidal concentration (MBC) on Gardnerella cells of the strains also used for the endolysin activity assays were then measured using the method described in Reference Example 6 and 7. Briefly, the measurement protocol had to be strongly adapted from the international standard, which is not suited for MIC and MBC measurements on Gardnerella. The main parameters to change were the growth medium (Gardnerella does not grow in Mueller-Hinton Broth usually used for MIC measurements), the anaerobic conditions, the time of incubation, and in the first round of experiments also the starting concentration of bacteria. The starting concentration was changed from the standard of 5×10⁵ CFU/ml to 2.5×10⁷ CFU/ml, mainly because also in the vagina of a BV patient, the cells are very concentrated, and the effect of the antibiotic should be measured at cell densities more comparable to ones used for the endolysin activity assays. The effect of Metronidazole (obtained from Gatt-Koller) and Clindamycin (obtained form Ratiopharm) on the growth in suspension of the Gardnerella strains was assessed. Essentially, Gardnerella suspensions at 2.5×10⁷ CFU/ml were incubated with the concentration of antibiotics as indicated and incubated for 48 h at 37° C. under anaerobic conditions. MIC was defined as the minimal concentration of antibiotic at which no growth was detectable after 48 h by OD measurement. OD(610) was measured at the beginning and the end of the experiment. At the end of the experiment, 2 ul of each reaction mix was spotted on agar to determine the MBC. Table 6A summarizes the results of the experiment described in FIG. 14. Resistance (R) defined as >=32 μg/mi for Metronidazole and >8 mg/mi for Clindamycin. Sensitivity (S) is defined as <=8 μg/ml for Metronidazole and <=2 μg/ml for Clindamycin, according to international standards.

TABLE 6A

| | Metronidazole | | Clindamycin | |
|---|---|---|---|---|
| | MIC | MBC | MIC | MBC |
| G. vaginalis (Gv_9) | 128 ug/ml-R | >128 ug/ml | 16 ug/ml-R | 128 ug/ml |
| G. leopoldii (Gv_11) | >128 ug/ml-R | >128 ug/ml | 16 ug/ml-R | 32 ug/ml |
| G. piotii (Gv_17) | 64 ug/ml-R | >128 ug/ml | 16 ug/ml-R | 64 ug/ml |
| G. swidsinskii | >128 ug/ml-R | >128 ug/ml | 16 ug/ml-R | >128 ug/ml |
| (Gv_23) | | | | |

TABLE 6B

| | Metronidazole | | Clindampycin | |
|---|---|---|---|---|
| | [μg/ml] | | [μg/ml] | |
| | MIC | MBC | MIC | MBC |
| G. vaginalis (Gv_9) | 8 | 16 (R) | 0.25 | 0.5 |
| G. leopoldii (Gv_11) | 128 (R) | >128 (R) | 0.5 | 1 |
| G. piotii (GV_17) | 16 | 64 (R) | 0.25 | 1 |
| G. swidsinskii (Gv_23) | 32 (R) | >128 (R) | 0.25 | 0.25 |

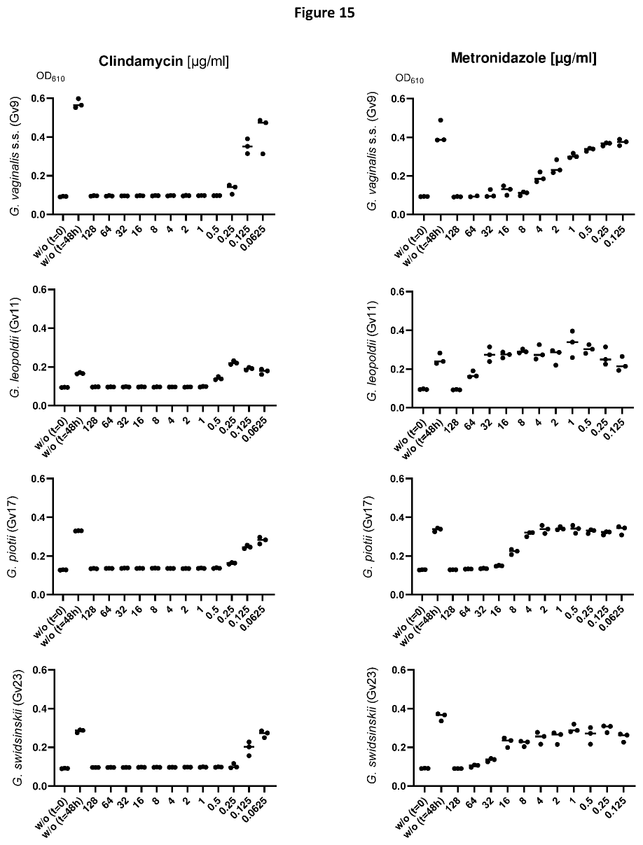

According to the results displayed in FIG. 14, all Gardnerella strains have a low susceptibility both to Metronidazole and Clindamycin. The conditions under which MIC and MBC were measured are more rigorous than the standard. For example usually, the MBC₉₀ is measured, i.e. the antibiotic concentration killing 90% of cells within a defined time, while MBC has been defined in the present application as the minimal concentration fully eradicating a suspension of 2.5×10⁷ CFU/ml. Nevertheless, these conditions are more comparable to what is found in the vagina of a BV patient. The high MIC and MBC values measured under these conditions could explain the high recurrence rates of BV. The assayed endolysins in contrast are bactericidal by definition, since they lead to complete disintegration of the bacterial cell. These results therefore sustain that the endolysins of the invention are superior to antibiotics in the treatment of BV.
A second round of MIC and MBC experiments was performed, where some experimental parameters were changed (i) the starting number of cells of 1×10⁵-1×10⁶ was now in accordance with the CLSI (Clinical and Laboratory Standards Institute) standards, (ii) Clindamycin hydrochloride powder (Sigma Aldrich, cat. no. C5269) was used (iii), NYC-III broth instead of Hardy broth, and (iv) 96-well plate instead of 384-well plates. As displayed in FIG. 15 and summarized in Table 6B, all Gardnerella strains still have a very low MIC to Metronidazole (8-128 μg/ml), whereas Clindamycin hydrochloride—in contrast to Clindamycin presented in FIG. 14 and Table 6A—was now inhibitory and bactericidal at low concentration (MIC<1 μg/ml).

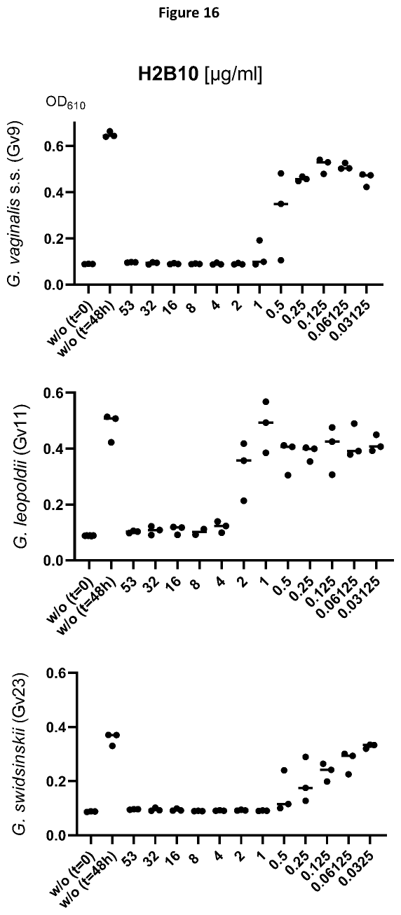

Example 7—Activity assays of a representative (H2B10) of domain swapped endolysins on the growth in suspension of different Gardnerella strains. For the analysis of MIC and MBC with the endolysin H2B10 cells suspensions of 1×10⁵-1×10⁶ were used. H2B10 showed a MIC in the low μg/ml range (0.5-4 μg/ml) indicating that Gardnerella cells are highly sensitivity towards endolysins (FIG. 16, Table 7). The conditions under which MBC was measured are more rigorous than the standard. For example usually, the MBC₉₀ is measured, i.e. the antimicrobial concentration killing 90% of cells within a defined time, while MBC has been defined in the present application as the dose that reduced the starting cell number by at least 99.5%. H2B10, as a representative of the herein claimed endolysins, showed a vastly superior MIC and MBC over the standard of care antibiotic Metronidazole, which is ineffective on many Gardnerella strains due to resistance formation. Clindamycin, however, gave inconsistent results. According to the international standards all four Gardnerella strains were supposed to be resistant (MIC>8 μg/ml) to Clindamycin, which was obtained from Ratiopharm (FIG. 14), whereas Clindamycin hydrochloride (Sigma Aldrich) was much more effective with bactericidal effects already at concentrations≤1 μg/ml (FIG. 16). In general, it is known, that antibiotics only insufficiently eradicate the Gardnerella biofilm, a hallmark of BV, which is one suspected reason for the reported very high recurrence rate of BV. Despite leaving residual viable biofilm, antibiotics wipe parts of the beneficial organisms of the vaginal microbiome which then opens an ecological niche for other pathogens, e.g. fungi. Thus, endolysin-based treatment that selectively eradicates bacterial cells of the genus Gardnerella and presumably eradicates the biofilm without harming the beneficial Lactobacilli is supposed to be superior to standard antibiotics therapy of BV.

TABLE 7

| | H2B10 | |
|---|---|---|
| | [μg/ml] | |
| | MIC | MBC |
| G. vaginalis (Gv_9) | 1 | 2 |
| G. leopoldii (Gv_11) | 4 | 16 |
| G. swidsinskii | 0.5 | 1 |
| (GV_23) | | |

### Reference Example 1—Cloning, Expression and Purification of Endolysins

### Materials:

96-well Multiscreen HTS Durapore 96-well Filterplatten, PS (Labshop cat. no. 44.MSGVS22)
PD MiniTrap desalting columns with Sephadex G-25 (GE Lifescience, cat. no. 28918007)
Slide-A-Lyzer™ MINI Dialysis Device, 10K MWCO, 2 mL (Thermo Scientific, cat. no. 88404)
Fastbreak reagent (Promega, cat. no. V8571)
Lysis Buffer: 50 mM Phosphate pH 6, 150 mM NaCl, 20 mM Imidazole, 1 mM TCEP, 1× FastBreak, Benzonase.
Wash Buffer I: 50 mM Phosphate pH 6, 150 mM NaCl, 20 mM Imidazole, 1 mM TCEP (1.5 ml)
Wash Buffer II: 50 mM Phosphate pH 6, 150 mM NaCl, 40 mM Imidazole, 1 mM TCEP (1.5 ml)
Elution Buffer: 50 mM Phosphate pH 6, 150 mM NaCl, 250 mM Imidazole, 1 mM TCEP (1.1 ml).

### Method:

Expression constructs were transformed into E. coli strain Bl21(DE3) and selected using appropriate antibiotics. Cells from 2 ml of culture (TB+Lactose, 25° C., O/N) were resuspended in 1.5 ml Lysis Buffer and lysed by FastBreak reagent (Promega). The intracellular soluble fraction was isolated by centrifugation at 15000 g, 30 min, 4° C. The soluble protein fraction was loaded onto 100 μL of Nickel affinity matrix, washed with 15 column volumes (CV) of Wash Buffers I and II each, and eluted in 10 CV elution buffer. Then, the eluate buffer might be exchanged to 20 mM phosphate pH 6.0, 150 mM NaCl, to remove imidazole, using desalting columns. After elution (or buffer exchange as appropriate), the concentration of the purified protein was adjusted to 0.2 mg/ml, then the solutions were sterile filtered using a 96-well filter plate.

### Reference Example 2—Activity Assays in Bacterial Suspensions

### Materials:

1. Hardy Broth, autoclaved 20 min at 121° C.:
12.0 g of Pancreatic Digest of Casein (SigmaAldrich, cat. no. 70172-100G)
10.0 g of Proteose Peptone (SigmaAldrich, cat. no. 82450-100G)
5.0 g of Peptic Digest of Animal Tissue (SigmaAldrich, cat. no. 70174-100G)
5.0 g of Sodium Chloride (CarlRoth, cat. no. 3957.1)
3.0 g of Beef Extract (SigmaAldrich, cat. no. B4888-50G)
3.0 g of Yeast Extract (SigmaAldrich, cat. no. Y1625-250G)
1.0 g of Soluble Starch (Sigma Aldrich, cat. no. S9765-250G)
deionized H2O to 1 liter (produced in the PhagoMed Lab with Millipore RiOs Essential 16)
   2. Hardy Broth Agar, autoclaved 20 min at 121° C.: Same as Hardy Broth, but with 15 g Agar Bacteriogical (OXOID Cat. #LP0011)
   3. Hardy Broth Top Agar, autoclaved 20 min at 121° C.: Same as Hardy Broth, but with 7 g Agar Bacteriogical (OXOID Cat. #LP0011)
   4. NYC-III medium, pH 5.0, autoclaved 20 min at 121° C., after which horse serum is added (NYC-III-HS-5.0)
12 g HEPES (Sigma Life Science, cat. no. H4034-100G)
7.5 g Proteose Peptone No. 3 (BD, cat. no. 211693)
1.9 g Yeast Extract (Sigma Aldrich, cat. no. Y1625-250G)
2.5 g Sodium Chloride (Sigma Aldrich, cat. no. S9888-1 kg-M)
2.5 g Glucose (MW 180.16 g/mol) (Sigma Aldrich, cat. no. G6152-1KG)
deionized water to 450 ml total volume
50 ml Horse Serum (HS), heat inactivated 100 ml (Thermo Fisher Scientific, cat. no. 26050070), added after autoclaving
   5. General materials:
BD Chocolate agar plates for Gardnerella (BD, cat. no. 254060)
BD Schaedler/5% sheep blood plates for Lactobacilli (BD, cat. no. 254042)
Isovitalex (BD, cat. no. 211876)
Hardy broth+Isovitalex (see above), adjusted to pH as indicated
Hardy agar+Isovitalex (see above)
Hardy top agar+Isovitalex (see above)
96-U-well plate (Sigma Aldrich, cat. no. M2311-100EA)
96 flat-bottom plate with lid (Labshop, cat. no. 44.781662)
Greiner CELLSTAR® 384 well plates (Sigma-Aldrich, cat. no. M1937-32EA)
Anaerogen sachets (Sigma-Aldrich, cat. no. 68061-10SACHETS-F)
Anaerobe indicator test (Sigma-Aldrich, cat. no. 59886-1PAK-F)
Anaerobic jar (Sigma-Aldrich, cat. no. 28029-1EA-F) or a plastic lunch box sealable with a rubber gasket, purchased at a local home appliances store
   6. Bacterial strains:
   Gardnerella strains:
Gv_1: UGent 09.07
Gv_8: UGent 25.49
Gv_9: ATCC 14018
Gv_10: UGent 06.41
Gv_11: UGent 09.48
Gv_17: UGent 18.01
Gv_23: GS 10234 (FC2)
   Lactobacilli strains:
L. jensenii PB2003-013-T2-2
L. gasseri 020566
L. crispatus LAB117

### Method:

Gardnerella cells were recovered from cryo stock by plating on Chocolate Agar plates (Beckton Dickinson) and incubating for 48 h at 37° C. under anaerobic conditions. For Lactobacilli, BD Schaedler/5% sheep blood agar plates were used instead. Colonies were scraped from the plate, resuspended in Hardy Broth or NYC-III-HS-5.0 at the pH as indicated, and the suspension adjusted to OD (610 or 620 nm as indicated) 0.1. It has to be noted that two Tecan Microplate readers, having respectively a 610 nm or 620 nm filter, have been used interchangeably in the experiments. Although it doesn't make any difference for the experiments, the exact wavelength used is specified in each example. If not stated otherwise, 90 μl cell suspension was mixed with 10 μl endolysin solution, for the different species/endolysin combinations, in 384-well plates. OD(610-620 nm as indicated) was measured at the beginning of the reaction and at the end, either as two measurement points or as a continuous kinetic in a Tecan F200 Microplate reader. The reactions were incubated for 5 hours (or otherwise the time indicated) at 37° C. under anaerobic, micro-aerophilic or aerobic conditions as indicated. Anaerobic conditions intend that oxygen was fully depleted from the container in which the bacteria are incubated (Sigma-Aldrich anaerobic jar or sealable lunch box) with an anaerobic sachet, and the lack of oxygen was confirmed with an anaerobic indicator inside the container. Where micro-aerophilic conditions are indicated, the candle-in-a-jar method was used (tea candle lit in an appropriate sealable container, which reduces oxygen levels until the flame dies out). Then each well was diluted in 5 steps (10⁻¹ to 10⁻⁵) using 96-U-well bottom plates, and 2 μl of each dilution of each reaction mix are plated on BD Chocolate agar plates or BD Schaedler/5% sheep blood agar plates for Gardnerella and Lactobacilli, respectively, for detecting and quantifying surviving CFU. Detection plates were incubated at 37° C. for 48 hours under anaerobic conditions.

### Reference Example 6 and 7—MIC and MBC Measurements

### Materials:

### General Materials:

Metronidazole (Gatt-Koller, Metronidazolum mikronisiert, 10 g, 606293914)
Clindamycin (Ratiopharm, 300 mg/2 ml ampulles 5×)
Clindamycin hydrochloride (Sigma Aldrich, 10 mg, cat. no C5269)
Endolysin H2B10 [530 μg/ml] in MES buffer (50 mM MES, 100 mM NaCl, 8 mM MgSO₄, pH=5.5)
BD Chocolate agar plates for Gardnerella (BD, cat. no. 254060)
BD Schaedler/5% sheep blood plates for Lactobacilli (BD, cat. no. 254042)
Isovitalex (BD, cat. no. 211876)
Hardy broth+Isovitalex (see above), adjusted to pH as indicated
NYC-III+HS broth. pH 5.5NYC-III-HS Agar plates: NYC-III+HS medium as described above, but with 1.5% Agar added prior to autoclaving
Hardy agar+Isovitalex (see above)
Hardy top agar+Isovitalex (see above)
96-U-well plate (Sigma Aldrich, cat. no. M2311-100EA)
96 flat-bottom plate with lid (Labshop, cat. no. 44.781662)
Greiner CELLSTAR® 384 well plates (Sigma-Aldrich, cat. no. M1937-32EA)
Anaerogen sachets (Sigma-Aldrich, cat. no. 68061-10SACHETS-F)
Anaerobe indicator test (Sigma-Aldrich, cat. no. 59886-1PAK-F)
Anaerobic jar (Sigma-Aldrich, cat. no. 28029-1EA-F) or a plastic lunch box sealable with a rubber gasket, purchased at a local home appliances store

### Method:

Bacteria were plated from cryo stock on BD Choc Agar plates (Gardnerella) and incubated at 37° C. for 48 h under anaerobic conditions. Colonies were scraped from the plate, resuspended in Hardy Broth or NYC-III-HS-pH 5.0, and the suspension adjusted to OD (610 or 620 nm as indicated) 0.05. It has to be noted that two Tecan Microplate readers, having respectively a 610 nm or 620 nm filter, have been used interchangeably in the experiments. Although it doesn't make any difference for the experiments, the exact wavelength used is specified in each example. Antibiotics were prepared as 20× stocks for each of the required final concentrations. 95 μl of cell suspension was mixed with 5 μl antibiotics dilution in a 384-well plate. OD (610-620 as indicated) at the start of the reaction was measured, then the plate was incubated at 37° C. for 48 h under anaerobic conditions. After that, the OD (610-620 as indicated) was measured again for MIC determination, where MIC was defined as the lowest concentration of antibiotic where the OD (610-620 as indicated) was not above the level measured at the beginning of the experiment. After measuring OD, 2 μl of each well were spotted on a NYC-III+HS Agar plate. The plates were then incubated for further 48 h at 37° C. under anaerobic conditions. After incubation, cell growth on each spot was evaluated, and the MBC defined as the lowest concentration of antibiotics where no bacteria grew on the plate. The experiments were conducted in triplicate for each condition.
In the second round of MIC and MBC experiments with antibiotics the Gardnerella cell suspension was adjusted to the McFarland standard 0.5 (approximately OD (610) 0.07) and then diluted 1:75 according to the CLSI (Clinical and Laboratory Standards Institute) standards. Antibiotics were prepared according to the CLSI standards and 50 μl of cell suspension was mixed in a 96-well plate with 50 μl antibiotics. Otherwise the MIC and MBC was determined as described above.
For the MIC and MBC determination of the domain swapped endolysin H2B10 50 μl of Gardnerella cell suspension was mixed in a 96-well plate with 50 μl of H2B10 containing solution, which where serially diluted 1:1. OD₆₁₀ at the start of the reaction was measured, then the plate was incubated at 37° C. for 48 h under anaerobic conditions. After that, the OD(610) was measured again for MIC determination, where MIC was defined as the lowest concentration of H2B10 where the OD was not or only slightly above the level measured at the beginning of the experiment.

TABLE 7

| Natural endolysin | Structure from N-terminal to C-terminal* | | | |
|---|---|---|---|---|
| EL1 | H1 | L1 | B1_N | B1_C |
| | SEQ ID NO: 1 | NAGLNGCKNGGS | SEQ ID NO: 15 | SEQ ID NO: 16 |
| EL2 | H2 | L2 | B2_N | B2_C |
| | SEQ ID NO: 2 | NVGLNGCKNGGS | SEQ ID NO: 17 | SEQ ID NO: 18 |
| EL3 | H3 | L3 | B3_N | B3_C |
| | SEQ ID NO: 3 | NAGLNGYQNGGS | SEQ ID NO: 19 | SEQ ID NO: 20 |
| EL4 | H4 | L4 | B4_N | B4_C |
| | SEQ ID NO: 4 | NVGLNGCKNGGS | SEQ ID NO: 21 | SEQ ID NO: 22 |
| EL5 | H5 | L5 | B5_N | B5_C |
| | SEQ ID NO: 5 | NAGLNGCKNGGS | SEQ ID NO: 23 | SEQ ID NO: 24 |
| EL6 | H6 | L6 | B6_N | |
| | SEQ ID NO: 6 | NAGLNGCKNGGS | SEQ ID NO: 25 | |
| EL7 | H7 | L7 | B7_N | B7_C |
| | SEQ ID NO: 7 | NAGLNGCKNGGS | SEQ ID NO: 26 | SEQ ID NO: 27 |
| EL8 | SEQ ID NO: 8 | | | |
| EL9 | SEQ ID NO: 9 | | | |
| EL10 | H10 | L10 | B10_N | B10_C |
| | SEQ ID NO: 10 | NAGLNGYKNGGS | SEQ ID NO: 28 | SEQ ID NO: 29 |
| EL11 | H11 | L11 | B11_N | B11_C |
| | SEQ ID NO: 11 | KAGLNGYKNGGS | SEQ ID NO: 30 | SEQ ID NO: 31 |
| EL12 | H12 | L12 | B12_N | B12_C |
| | SEQ ID NO: 12 | NAGLNGYQNGGS | SEQ ID NO: 32 | SEQ ID NO: 33 |

| | |
|---|---|
| EL13 | SEQ ID NO: 13 |
| EL14 | SEQ ID NO: 14 |

*1^{st} row = Name according to the nomenclature of the present application, if appropriate; 2^{nd} row = the corresponding native amino acid sequence

>H1
>196 aa
SEQ ID NO: 1
MSKKGIDVSEWQGDIDFNAVKASGVEFVIIRAGYGIGCKDKWFEQNYRKAK
TAGLDVGAYWYSYANSGFEAAEEAQSLMNMLSGKSFEYPVYFDLEEKSQLN
RGRAFCDSLITSFCNKLEACGYYAGFYTSLSTANNLVSAHVRNRYALWIAQ
WNTHCNYQGSYGLWQYSSNGSVPGVAGRVDMDYAYVDYPSIIK
>H2
>196 aa
SEQ ID NO: 2
MSKRGIDVSEWQGDIDFNAVKASGVEFVIIRAGYGIGCKDKWFEQNYRKAK
TCGLDVGAYWYSYANSGFEAAEEAQSCVNMLSGKSFEYPVYFDLEEKSQLN
RGRAFCDSLITGFCNKLESCGYYAGFYTSLSTANNLVSAHVRNRYALWIAQ
WNTHCSYQGSYGLWQYSSSGSVPGVAGRVDMDYAYVDYPSIIK
>H3
>196 aa
SEQ ID NO: 3
MSKKGIDVSVWQGDIDFNSVKASGVEFVIIRAGYGIGHKDKWFEENYRKAK
TAGLDVGSYWYSYASSAGEASEEAQSCVNILSGKSFEYPIYFDLEEKSQLN
RGRDFCDSLITSFCNKLEACGYYAGFYTSLSVANNLVSSHVRDRYALWIAQ
WNTHCSYQGSYGLWQYSSSGSVNGIAGRVDMDYAYVDYPSVIK
>H4
>196 aa
SEQ ID NO: 4
MSKKGIDVSEWQGDIDFNAVKASGVEFVIIRAGYGIGCKDKWFEQNYRKAK
TCGLDVGAYWYSYANSGFEAAEEAQSCVNMLSGKSFEYPVYFDLEEKSQLN
RGRAFCDSLITSFCSKLETYGYYAGFYTSLSVVNNLVSAHVRDRYALWIAQ
WNTHCSYQGSYGLWQYSSSGSVPGVAGRVDMDYAYVDYPSIIK
>H5
>196 aa
SEQ ID NO: 5
MSKKGIDVSEWQGDIDFNAVKASGVEFVIIRAGYGIGCKDKWFEQNYRKAK
TAGLDVGAYWYSYANSSSEAAEEAQSCANMLSGKSFEYPVYFDLEEKSQLN
RGRAFCDSLITGFCSKLEACGYYAGFYTSLSTANNLVSAHVRNRYALWIAQ
WNTHCSYQGSYGLWQYSSNGSVPGVAGRVDMDYAYKDYPSIIK
>H6
>196 aa
SEQ ID NO: 6
MSKKGIDVSVWQGDIDFNAVKASGVEFVIIRAGYGIGCKDKWFEQNYRKAK
TCGLDVGAYWYSYANSGFEAAEEAQSCVNMLSGKSFEYPVYFDLEEKSQLN
RGRAFCDSLITSFCSKLESCGYYAGFYTSLSTANNLVSAHVRNRYALWIAQ
WNTHCDYQGSYGLWQYSSSGSVPGVAGRVDMDYAYKNYPSIIK
>H7
>196 aa
SEQ ID NO: 7
MSKKGIDVSEWQGDIDFNAVKASGVEFVIIRAGYGIGCKDKWFEQNYRKAK
TAGLDVGAYWYSYANSASEAAEEAQSCANMLSGKSFEYPVYFDLEEKSQLN
RGRAFCDSLITSFCSKLETYGYYAGFYTSLSTANNLVSSHVRNRYALWIAQ
WNTHCSYQGSYGLWQYSSSGSVPGVAGRVDMDYAYKDYPSIIK
>EL8
>306 aa
SEQ ID NO: 8
MSKKGIDVSEWQGDIDFNAVKASGVEFVIIRAGYGIGCKDKWFEQNYRKAK
TAGLDVGAYWYSYANSSSEAAEEAQSCVNMLSGKSFEYPVYFDLEEKSQLN
RGRAFCDSLITSFCSKLETYGYYAGFYTSLSTANNLVSSHVRNRYALWIAQ
WNTHCSYQGSYGLWQYSSNGSVPGVAGRVDMDYAYVDYPSIIKNAGLNGYK
NGGSYTAPQTSSIDDVAREVINGAWGNGNERKQRLTQAGYDYTSVQNKVNK
LLGVKACRKSVDELAREVIRGTWGNGNERKNRLTQAGYDYDTVQKRVNELL
>EL9
>251 aa
SEQ ID NO: 9
MSKKGIDVSEWQGDIDFNAVKASGVEFVIIRAGYGIGCKDKWFEQNYRKAK
TCGLDVGAYWYSYANSGFEAAEEAQSCVNMLSGKSFEYPVYFDLEEKSQLN
RGRVFCDSLITSFCNKLEACGYYAGFYTSLSTANNLVSSHVRNRYALWIAQ
WNTHCDYQGSYGLWQYSSSGSVPGVAGRVDMDYAYVDYPSIIKNAGLNGCK
NGGSDQAARTSSIDEVAREVINGAWGNGSTRKQRLTSAGYDYASVAK
>H10
>196 aa
SEQ ID NO: 10
MSKRGIDVSVWQGDIDFNAVKASGVEFVIIRAGYGIGHKDKWFEENYRKAK
TVGLDVGAYWYSYASSAGEASEEAQSCVNILSGKSFEYPVYFDLEEKSQLN
RGRDFCDSLITSFCNKLEACGYYAGFYTSLSVANNLVSSHVRDRYALWIAQ
WNTHCSYQGSYGLWQYSSSGSVNGIAGRVDMDYAYVDYPSVIK
>H11
>196 aa
SEQ ID NO: 11
MSKRGIDVSVWQGDIDFNAVKASGVEFVIIRAGYGIGHKDKWFEQNYRKAK
TTGLDVGAYWYSYASSAGEAAEEAQSCVNILSGKSFEYPVYFDLEEKSQLN
RGRDFCDSLITSFCNKLETYGYYAGFYTSLSVANNLVSSHVRDRYALWIAQ
WNTHCDYQGSYGLWQYSSSGSVDGIAGRVDMDYTYVDYPSVIK
>H12
>196 aa
SEQ ID NO: 12
MSKKGIDVSVWQGDIDFNAVKASGVEFVIIRAGYGIGHKDKWFEENYRKAK
TAGLDVGSYWYSYASSAGEVALEAQSCVNILSGKSFEYPVYFDLEEKSQLN
RGRDFCDSLITSFCNKLEACGYYAGFYTSLSVANNLVSSHVRDRYALWIAQ
WNTHCSYQGSYGLWQYSSSGSVNGIAGRVDMDYAYVDYPSVIK
>EL13
>306 aa
SEQ ID NO: 13
MSKKGIDVSVWQGDIDFNAVKASGVEFVIIRAGYGIECKDKWFEQNYRKAK
TAGLDVGAYWYSYANSGFEAAEEAQSCVNMLSGKSFEYPVYFDLEEKSQLN
RGRAFCDSLITSFCNKLESCGYYAGFYTSLSTANNLVPAHVRNRYALWIAQ
WNTHCDYQGSYGLWQYSSSGSVPGVAGRVDMDYAYVDYPSIIKNAGLNGYK
NGESHQATRTTSIDEVAREVINGAWGNGNERKQRLTQAGYDYASVQNKVNE
LLGVKACRKSVDELAREVIRGTWGNGNERKNRLTSAGYDYDTVQKRVNELL
>EL14
>306 aa
SEQ ID NO: 14
MSKKGIDVSVWQGDIDFNAVKASGVEFVIIRAGYGIGCKDKWFEQNYRKAK
TCGLDVGAYWYSYANSGFEAAEEAQSCVNMLSGKSFEYPVYFDLEEKSQLN
RGRAFCDSLITSFCNKLEACGYYAGFYTSLSTANNLVSAHVRNRYALWIAQ
WNTHCSYQGSYGLWQYSSSGSVPGVAGRVDMDYAYVDYPSIIKNAGLNGYK
NGESHQATRTTSIDEVAREVINGAWGNGNERKQRLTSAGYDYASVQNKVNE
LLGVKACRKSVDELAREVIRGAWGNGSTRKQRLTSAGYDYDTVQKRVNELL
>B1_N
>49 aa
SEQ ID NO: 15
DQAARTSSIDEVAREVINGAWGNGSTRKQRLTSAGYDYASVQNKVNELL
>B1_C
>49 aa
SEQ ID NO: 16
GVKACRKSVDELAREVIRGAWGNGSTRKQRLAQAGYDYDTVQKRVNELL
>B2_N
>49 aa
SEQ ID NO: 17
DQAARTSSIDEVAREVINGAWGNGNERKQRLTSAGYDYASVQNKVNELL
>B2_C
>49 aa
SEQ ID NO: 18
GVKACRKSVDEIAREVIRGTWGNGSTRKQRLTQAGYDYDTVQKRVNELL
>B3_N
>49 aa
SEQ ID NO: 19
YTAPQTSSIDEVAREVINGDWGNGNDRKNRLISAGYDYASVQNKVNELL
>B3_C
>49 aa
SEQ ID NO: 20
GVKAYRKSVDELAREVIRGTWGNGSMRKHRLTQAGYDYDAVQKRVNELL
>B4_N
>49 aa
SEQ ID NO: 21
DQATRISSIDEVAREVINGAWGNGNERKQRLTSAGYDYASVQNKVNKLL
>B4_C
>49 aa
SEQ ID NO: 22
GVKAYRKSVDELAREVIRGTWGNGNERKQRLAQAGYDYDTVQKRVNELL
>B5_N
>49 aa
SEQ ID NO: 23
DQAARTSSIDEVAREVINGAWGNGNERKQRLTQAGYDYTSVQNKVNKLL
>B5_C
>49 aa
SEQ ID NO: 24
GVKACRKSVDELAREVIRGTWGNGNERKNRLTQAGYDYDTVQKRVNELL
>B6_N
>43 aa
SEQ ID NO: 25
NQAARTSSIDDVAREVINGAWGNGNERKQRLTQAGYDYASVAK
>B7_N
>49 aa
SEQ ID NO: 26
DQAARTSSIDEVAREVINGAWGNGNERKQRLTQAGYDYTSVQNKVNKLL
>B7_C
>49 aa
SEQ ID NO: 27
GVKACRKSVDELAREVIRGTWGNGNERKNRLTQAGYDYDTVQKRVNELL
>B10_N
>49 aa
SEQ ID NO: 28
YTAPQISSIDEVAREVINGDWGNGNERKQRLTSAGYDYASVQNKVNELL
>B10_C
>49 aa
SEQ ID NO: 29
GVKAYRKSVDELAREVIRGTWGNGSTRKQRLTQAGYDYNAVQKRVNELL
>B11_N
>49 aa
SEQ ID NO: 30
YTAPQTSSIDEVAREVINGDWGNGNERKNRLTSAGYDYTSVQNKVNELL
>B11_C
>49 aa
SEQ ID NO: 31
GVKAYRKSVDELAREVIRGTWGNGSTRKQRLTQAGYDYDAVQKRVNELL
>B12_N
>49 aa
SEQ ID NO: 32
YTAPQTSSIDEVAREVINGDWGNGIERKNRLTSAGYDYTSVQNKVNELL
>B12_C
>49 aa
SEQ ID NO: 33
GVKAYRKSVDELAREVIRGTWGNGKTRKQRLTQAGYDYNAVQKRVNELL

1. A recombinant endolysin comprising:
(i) a N-terminal catalytic domain, or a functional variant thereof,
(ii) a C-terminal cell-wall binding region, or a functional variant thereof, wherein the C-terminal cell-wall binding region comprises or consists of one or more cell-wall binding domains, and
(iii) a linker region between the N-terminal catalytic domain and the C-terminal cell-wall binding region,
wherein the N-terminal catalytic domain is from a first natural endolysin, the linker region and the C-terminal cell-wall binding region are from a second natural endolysin, and wherein the first and the second natural endolysins are encoded by different genomes from different prophages, and
wherein said recombinant endolysin has a genus-selective killing activity against Gardnerella.

2. The recombinant endolysin of claim 1, wherein the N-terminal catalytic domain is a polypeptide comprising or consisting of the amino acid sequence of any one of SEQ ID NOs: 1 to 5, 7, or 10 to 12, or any variant thereof having at least 80% identity with the amino acid sequence of any one of SEQ ID NOs: 1 to 5, 7, or 10 to 12, whereby said polypeptide is functional, wherein the function comprises the ability to lyse the cell wall of Gardnerella.

3. The recombinant endolysin of claim 1, wherein the C-terminal cell-wall binding region comprises or consists of one, two or three cell-wall binding domains.

4. The recombinant endolysin of claim 3, wherein the one, two or three cell-wall binding domains are independently selected from the group consisting of the polypeptides comprising or consisting of the amino acid sequence of SEQ ID NOs: 15 to 24 and 26 to 33, respectively, and any variants thereof having at least 80% identity with the amino acid sequence of SEQ ID NOs: 15 to 24 and 26 to 33, respectively, whereby said polypeptides are functional, wherein the function comprises the ability to bind to the cell wall of Gardnerella.

5. The recombinant endolysin of claim 1, wherein the C-terminal cell-wall binding region comprises or consists of a first cell-wall binding domain and a second cell-wall binding domain, wherein said first cell-wall binding domain is selected from the group consisting of SEQ ID NOs: 15, 17, 19, 21, 23, 26, 28, 30 and 32 and said second cell-wall binding domain is selected from the group consisting of SEQ ID NOs: 16, 18, 20, 22, 24, 27, 29, 31 and 33.

6. The recombinant endolysin of claim 5, wherein said first cell-wall binding domain is N-terminally of said second cell-wall binding domain.

7. The recombinant endolysin of claim 1, wherein the linker region is a polypeptide comprising or consisting of the amino acid sequence:
(i) (XXX)n, wherein each X can be independently G, A or S, preferably wherein the amino acid sequence (XXX)n is (GGS)n, wherein n corresponds to the number of repetitions of the sequence XXX, preferably wherein n is 2, 3, 4, 5 or 6; or
(ii) X₁X₂GLNGX₃X₄NGGS, wherein X₁ is N or K, X₂ is A or V, X₃ is Y or C and X₄ is K or Q.

8. The recombinant endolysin of claim 1, wherein said endolysin has a killing activity against Gardnerella vaginalis sensu stricto, Gardnerella leopoldii, Gardnerella piotii and/or Gardnerella swidsinskii, or any other species in the genus Gardnerella.

9. The recombinant endolysin of claim 1, wherein said endolysin has no killing activity against Lactobacilli, preferably wherein said endolysin has no killing activity against Lactobacilli crispatus, Lactobacilli gasseri, and/or Lactobacilli jensenii.

10. A polynucleotide which encodes the recombinant endolysin of claim 1.

11. A pharmaceutical composition comprising the recombinant endolysin of claim 1 and further comprising a pharmaceutically acceptable carrier and/or diluent.

12. A method of treating a bacterial infection, comprising administering to a subject in need thereof a recombinant endolysin according to claim 1.

13. (canceled)

14. The method according to claim 12, wherein said bacterial infection is bacterial vaginosis.

15. The method according to claim 14, wherein said bacterial vaginosis is caused by Gardnerella vaginalis sensu stricto, Gardnerella leopoldii, Gardnerella piotii and/or Gardnerella swidsinskii.

16. The method according to claim 12, wherein said recombinant endolysin is administered locally.

17. The method according to claim 12, wherein said recombinant endolysin is administered into the vagina of a female subject and/or into or on the glans penis, prepuce or urethral entry of a male subject.

18. The method of claim 12, wherein said recombinant endolysin is co-administered with a compound or composition which adjusts the pH of the vagina to 4.0-6.0.

19. A plasmid comprising the polynucleotide of claim 10.

20. A bacterial host cell comprising the plasmid of claim 19, preferably wherein the bacterial host cell is an E. coli cell.

21. An in vitro method for the diagnosis of a disease or condition which can be treated with the endolysin according to claim 1, the method comprising the steps of:
(i) contacting a sample obtained from the subject with a polypeptide comprising or consisting of the C-terminal cell-wall binding region of the endolysin according to claim 1, and optionally the N-terminal catalytic domain of the endolysin according to claim 1, wherein the sample comprises microbial cells, and wherein the C-terminal cell-wall binding region of said endolysin is optionally labelled;
(ii) testing whether the polypeptide binds to, and/or lyses, the microbial cells of the sample; and
(iii) determining that a disease or condition can be treated with the endolysin according to claim 1 if the polypeptide binds to, and/or lyses, the microbial cells.
